(19) United States
(12) Patent Application Publication
(10) Pub. No.: US 2026/0124314 A1
(43) Pub. Date: May 7, 2026

(54) ANTl-5T4 ANTIGEN BINDING DOMAINS, ANTIBODY-DRUG CONJUGATES AND METHODS OF USE THEREOF

(71) Applicant: Salubris Biotherapeutics, Inc., Gaithersburg, MD (US)

(72) Inventors: Samuel L. MURPHY, Cambridge, MA (US); Jonathan P. MCNALLY, Charles Town, WV (US); Binyam Z. BEZABEH, Silver Spring, MD (US); John LI, Reston, VA (US)

(21) Appl. No.: 19/333,865

(22) Filed: Sep. 19, 2025

Related U.S. Application Data
(63) Continuation of application No. PCT/US2024/020929, filed on Mar. 21, 2024.
(60) Provisional application No. 63/453,929, filed on Mar. 22, 2023.

(51) Int. Cl. A61K 47/68; A61P 35/00; C07K 16/30

(52) U.S. Cl. CPC A61K 47/68031; A61K 47/6851; A61P 35/00; C07K 16/30; C07K 2317/31; C07K 2317/34; C07K 2317/73; C07K 2317/77; C07K 2317/92

(57) ABSTRACT

The disclosure provides anti-5T4 antigen binding domains, which can be incorporated into antibodies and receptors, including bispecific anti-5T4 antibodies, biparatopic anti-5T4 antibodies, and antibody-drug conjugates of same comprising a first antigen binding domain that specifically binds to a first 5T4 epitope, a second antibody antigen binding domain that specifically binds to a second 5T4 epitope that is not the same as the first 5T4 epitope, wherein the first antigen binding domain is operably linked to the second antigen binding domain, and a chemotherapeutic agent. The disclosure further provides methods of using same for the treatment of cancer.

### RELATED APPLICATIONS

This application is a continuation of International Patent Application No. PCT/US2024/020929, filed on Mar. 21, 2024, which claims priority to, and the benefit of, U.S. Provisional Application No. 63/453,929, filed on Mar. 22, 2023, the contents of each of which are incorporated herein by reference in their entireties.

### REFERENCE TO AN ELECTRONIC SEQUENCE LISTING

The contents of the electronic sequence listing (SBTI-005_C01US_SeqList_ST26.xml; Size: 163,185 bytes in size; and Date of Creation: Sep. 8, 2025) are herein incorporated by reference in its entirety.

### BACKGROUND

Cancer is one of the leading causes of death in the developed world. In the United States alone, an estimated 1.8 million people were newly diagnosed, and over 600,000 cancer deaths occurred in 2020. In cancer, cells of the subject grow and divide abnormally, spreading into surrounding tissues. Each cancer is thought to have combination of genetic changes, which may vary between cancers that allow cancer cells to escape the body's natural controls on cellular proliferation and allow the cancer to spread. While some cancers are currently treatable, many cancers are not. There exists a need in the art for compositions and methods for the treatment of cancer.

Human 5T4 is manifested in a variety of cancer types, including bladder cancer, breast cancer, cervical cancer, endometrial cancer, lung cancer, esophageal cancer, ovarian cancer, pancreatic cancer, gastric cancer, and testicular cancer. Human 5T4 is not generally found in normal tissues, and where found it is expressed at low levels, making it an ideal therapeutic target for the treatment of cancer. The present disclosure provides antibodies and antibody-drug conjugates comprising chemotherapeutic agents that specifically bind to a first and in some cases a second 5T4 epitope, compositions comprising the same, and methods of making and using the same for the treatment of diseases such as cancer.

### SUMMARY

The disclosure provides 5T4 antigen binding domains, as well as antibodies, antibody-drug conjugates, and receptors comprising same.

The disclosure provides 5T4 biparatopic antibody-drug conjugates, comprising: (a) a first antigen binding domain that specifically binds to a first 5T4 epitope; (b) an antigen binding domain that specifically binds to a second 5T4 epitope that is not the same as the first 5T4 epitope; and (c) a chemotherapeutic agent; wherein the first antigen binding domain is operably linked to the second antigen binding domain. In some embodiments of the 5T4 biparatopic antibodies and antibody-drug conjugates described herein, the biparatopic antibody or antibody-drug conjugate comprises a full length IgG antibody comprising the first antigen binding domain, and the second antigen binding domain comprises an scFv.

In some embodiments of the antibody-drug conjugates of the disclosure, the N-terminus of the second antigen binding domain is operably linked to the C-terminus of a heavy chain of the first antigen binding domain. In some embodiments, the C-terminus of the second antigen binding domain is operably linked to the N-terminus of a heavy chain of the first antigen binding domain. In some embodiments, the second antigen binding domain is operably linked to the heavy chain of the first antigen binding domain using a linker. In some embodiments, the antibody or antibody-drug conjugate comprises a full-length IgG antibody comprising the first antigen binding domain, and the second antigen binding domain comprises an scFv, and the antibody or antibody-drug conjugate comprises four polypeptides comprising: (a) two polypeptides comprising, from N to C terminus, the first antigen binding domain heavy chain, a linker, and the second antigen binding domain; and (b) two polypeptides comprising the first antigen binding domain light chain. In some embodiments, antibody or antibody-drug conjugate comprises a full-length IgG antibody comprising the first antigen binding domain and the second antigen binding domain comprises an scFv, and the antibody-drug conjugate comprises four polypeptides comprising: (a) two polypeptides comprising, from N to C terminus, the second antigen binding domain, a linker, and the first antigen binding domain heavy chain; and (b) two polypeptides comprising the first antigen binding domain light chain.

In some embodiments, the chemotherapeutic agent is an auristatin, for example an auristatin selected from the group consisting of auristatin E (AE), monomethyl auristatin D (MMAD), monomethyl auristatin E (MMAE), monomethyl auristatin F (MMAF), and synthetic analogs of dolastatin.

The disclosure provides nucleic acid systems and vectors encoding the antigen binding domains, antibodies and receptors of the disclosure.

The disclosure provides pharmaceutical compositions comprising the antigen binding domains, antibodies, antibody-drug conjugates, and immune cells comprising receptors of the disclosure.

The disclosure provides pharmaceutical compositions comprising the antigen binding domains, antibodies, antibody-drug conjugates, and immune cells comprising receptors of the disclosure, for use in the treatment of cancer in a subject.

The disclosure provides pharmaceutical compositions comprising the antigen binding domains, antibodies, antibody-drug conjugates, and immune cells comprising receptors of the disclosure, for use in the manufacture of a medicament for the treatment of cancer in a subject.

The disclosure provides methods of treating cancer in a subject in need thereof, the method comprising administering to a subject in need thereof a therapeutically effective amount of an antigen binding domain, antibody, antibody-drug conjugate, or immune cells a comprising receptor of the disclosure. In some embodiments, the methods comprise administering an antibody-drug conjugate comprising (a) a first antigen binding domain that specifically binds to a first 5T4 epitope; (b) a second antigen binding domain that specifically binds to a second 5T4 epitope that is not the same as the first 5T4 epitope; and (c) a chemotherapeutic agent; wherein the first antigen binding domain is operably linked to the second antigen binding domain.

The disclosure provides methods of manufacturing antibody-drug conjugates, the method comprising: (a) culturing a cell comprising a nucleic acid system that encodes the antibody under conditions that lead to expression of the antibody, (b) recovering the antibody; and (c) conjugating the antibody to a chemotherapeutic agent.

### BRIEF DESCRIPTION OF THE DRAWINGS

FIGS. 1A-1B are schematics depicting the structural configuration of exemplary anti-5T4 biparatopic antibody-drug conjugates. FIG. 1A shows a first exemplary antibody-drug conjugate (Bs2 orientation). FIG. 1B shows a second exemplary antibody-drug conjugate (Bs3 orientation). VH: variable heavy chain; VL: variable light chain; scFv: single chain variable fragment.

FIGS. 2A-2C show graphs and a table depicting size exclusion chromatography analysis after protein A purification of four exemplary anti-5T4 biparatopic antibodies. FIG. 2A depicts two graphs showing monomeric peak analysis of two exemplary antibodies in the Bs2 orientation shown in FIG. 1A. The left plot shows an antibody with the scFv in the VL-VH orientation, while the right plot shows an antibody with the scFv in the VH-VL orientation. FIG. 2B depicts two graphs showing monomeric peak analysis of two exemplary antibodies in the Bs3 orientation shown in FIG. 1B. The left plot shows an antibody with the scFv in the VL-VH orientation, while the right plot shows an antibody with the scFv in the VH-VL orientation. FIG. 2C is a table showing quantitative soluble protein yield, in mg/L, and relative purity, by size exclusion chromatography (SEC) peak, for the antibody variations depicted in FIGS. 2A-2B from a 10 day culture harvest.

FIGS. 3A-3C are two graphs and a table depicting an SPR-based binding assay to confirm that the 5T4 epitopes bound by antibodies 1 and 2 are non-competitive. FIG. 3A shows an assay in which mouse Ab1 ((m)Ab1) was captured on the sensor chip using anti-mouse IgG antibodies, followed by injection of 5T4, then humanized Ab1 or Ab2 with human Fc. FIG. 3B shows an assay in which mouse Ab2 ((m)Ab2) was captured on the sensor chip using anti-mouse IgG antibodies, followed by injection of 5T4, then humanized Ab1 or Ab2 with human Fc. FIG. 3C is a table showing the legend showing the binding sensorgram and injection samples for FIGS. 3A-3B. The y-axis in FIGS. 3A and 3B indicates relative response (in resonance units, or RU), while the x-axis indicates time (in seconds, or s).

FIGS. 4A-4E show the binding affinity of parental monospecific antibodies and biparatopic antibodies to 5T4 as determined by Biacore analysis. FIG. 4A shows the binding of a first exemplary monospecific antibody, humanized antibody Ab1. FIG. 4B shows the binding of a second exemplary monospecific antibody, humanized antibody Ab2. FIG. 4C shows the binding of an exemplary biparatopic antibody in the Bs3 orientation with the scFv in the VH-VL orientation (Bs3-HL). FIG. 4D shows the binding of a second exemplary biparatopic antibody (Bs3-HL-FCA, Bs3 with the scFv in the VH-VL orientation, and with the L234F, S239C and N434A mutations). FIG. 4E is a table summarizing the equilibrium dissociation constants (KD), dissociation rate constants (Kd) and association rate constants (Ka) of the antibody binding assays from FIGS. 4A-4D. For FIGS. 4A-4D, the y axis shows response (in RU), and the x-axis shows time (in seconds).

FIG. 5 is a table showing percent sequence identity of 5T4 proteins from rhesus monkey, cynomolgus monkey, mouse and rat to human 5T4.

FIG. 6 is a graph depicting binding of an exemplary biparatopic 5T4 antibody in the Bs3 orientation to 5T4 protein from various species using an ELISA assay. NHP: non-human primate.

FIGS. 7A-7D are a series of graphs depicting binding specificity of an exemplary biparatopic antibody in the Bs3 orientation to 5T4 protein from various species as determined by surface plasmon resonance (SPR). FIG. 7A shows binding to human 5T4. FIG. 7B shows binding to NHP 5T4. NHP: non-human primate. FIG. 7C shows binding to mouse 5T4. FIG. 7D shows binding to rat 5T4. The y-axis in all of FIGS. 7A-7D indicates relative response (in RU), while the x-axis indicates time (in seconds). In FIGS. 7A-7D, antibody concentrations are as follows: 100 nM (purple), 33.3 nM (yellow), 11.1 nM (pink), 3.7 nM (green), 1.2 nM (orange) and 133 pM (blue).

FIG. 8 is a table depicting the summary of equilibrium dissociation constants (KD) for binding of an exemplary biparatopic antibody in the Bs3 HL format with Fc mutation shown in FIG. 1A and a parental Ab1 without the Fc mutations to select Fc gamma receptors (FcγRs). FCA: L234F, S239C and N434A mutations.

FIGS. 9A-9L show a series of plots depicting flow cytometry analysis of cell surface 5T4 expression in a panel of 5T4-negative and 5T4-positive cell lines. FIG. 9A shows AGS cells. FIG. 9B shows HepG2 cells. FIG. 9C shows LoVo cells. FIG. 9D shows PCI-N87 cells. FIG. 9E shows A549 cells. FIG. 9F shows DU145 cells. FIG. 9G shows PANC-1 cells. FIG. 9H shows T-47D cells. FIG. 9I shows MCF7 cells. FIG. 9J shows HEK293-5T4 (3G9) cells. FIG. 9K shows HEK293-5T4 (4F2) cells. FIG. 9L shows HEK293-5T4 (5C10) cells.

FIGS. 10A-10F are a series of graphs depicting 5T4 receptor internalization induced by biparatopic antibodies in the Bs3-HL and Bs2-HL formats, with or without the FCA (L234F, S239C and N434A) mutations, compared with parental monospecific antibodies (humanized versions of antibodies Ab1 and Ab2). 5T4 receptor internalization was assayed in multiple 5T4-expressing cell types. FIG. 10A shows DU145 cells. FIG. 10B shows PANC-1 cells. FIG. 10C shows MCF7 cells. FIG. 10D shows HEK293-5T4 (3G9) cells. FIG. 10E shows HEK293-5T4 (4F2) cells. FIG. 10F shows T-47D cells.

FIGS. 11A-11F are a series of graphs depicting mean percent viability of cancer cell lines incubated with biparatopic antibodies in the Bs3-HL format conjugated to MMAE, compared to an IgG antibody not specific to 5T4 (IgG-Ctrl) conjugated to MMAE, and MMAE (no antibody) controls. Viability was assayed in multiple 5T4-expressing cell types. FIG. 11A shows AGS cells. FIG. 11B shows DU145 cells. FIG. 11C shows T-47D cells. FIG. 11D shows MCF7 cells. FIG. 11E shows HEK293-5T4 (3G9) cells. FIG. 11F shows HEK293-5T4 (4F2) cells. cc4 and cc8 indicate different drug to antibody ratios (DARs), of about 4 and 8 respectively. The y-axis shows percent viability, while the x-axis indicates concentration of antibody-drug conjugate (in pM). MMAE: monomethyl auristatin E.

FIGS. 12A-12F are a series of graphs depicting mean percent growth inhibition of cancer cell lines incubated with biparatopic 5T4 antibodies in the Bs3-HL format conjugated to MMAE, compared to an IgG antibody not specific to 5T4 (IgG-Ctrl) conjugated to MMAE and MMAE (no antibody) controls. Growth inhibition was assayed in multiple 5T4-expressing cell types. FIG. 12A shows AGS cells. FIG. 12B shows DU145 cells. FIG. 12C shows MCF7 cells. FIG. 12D shows T-47D cells. FIG. 12E shows HEK293-5T4 (3G9) cells. FIG. 12F shows HEK293-5T4 (4F2) cells. cc4 and cc8 indicate different drug to antibody ratios (DARs), of about 4 and 8 respectively. The y-axis shows percent growth inhibition, while the x-axis indicates concentration of antibody-drug conjugate (in pM). MMAE; monomethyl auristatin E.

FIGS. 13A-13B are two graphs depicting tumor growth and mouse survival in immune compromised mice implanted with NCI-H1975 lung adenocarcinoma tumors and treated with 5T4 biparatopic antibody in the Bs3 orientation, conjugated to MMAE (2 dose concentrations), compared to isotype control antibody conjugated to MMAE and mock PBS control. FIG. 13A shows tumor growth. FIG. 13B shows probability of survival. ADC: antibody-drug conjugate; MMAE; monomethyl auristatin E.

FIGS. 14A-14C are a series of graphs depicting tumor growth in immune compromised mice implanted with various CDX tumor models and treated with 5T4 biparatopic antibody in the Bs3 orientation, conjugated to MMAE (multiple concentrations), compared to isotype control antibody conjugated to MMAE or mock PBS control. FIG. 14A shows a MDA-MD-361 breast cancer model. FIG. 14B shows a DU145 prostate carcinoma model. FIG. 14C shows a A549 lung carcinoma model. ADC: antibody-drug conjugate; MMAE; monomethyl auristatin E.

FIGS. 15A-15D are a series of sensorgrams depicting binding affinity of 5T4 parental antibodies compared to 5T4 biparatopic antibodies in the Bs3 configuration. FIG. 15A shows binding affinity of parental antibody (m)Ab1. FIG. 15B shows binding affinity of parental antibody (m)Ab2. FIG. 15C shows binding affinity of the bispecific antibody in the Bs3-HL orientation. FIG. 15D shows binding affinity of bispecific antibody in the Bs3-HL orientation conjugated to MMAE; monomethyl auristatin E.

FIG. 16 is a table depicting the summary of equilibrium dissociation constants (KD) for binding of 5T4 antibodies as shown in FIGS. 15A-15D, with and without MMAE conjugation, to 5T4.

### DETAILED DESCRIPTION

The disclosure provides antigen binding domains that specifically bind to 5T4, and methods of making and using same. 5T4 antigen binding domains of the disclosure include, but are not limited to, Fab fragments, F(ab′)₂ fragments, scFv, scab, dAb, single domain antibodies, full length IgG antibodies and the like. Non-limiting uses of the 5T4 antigen binding domains contemplated as within the scope of the instant disclosure include immunotherapies, use as antibody-drug conjugates, and incorporation into chimeric antigen receptors (CARs) used in adoptive cell therapies.

Accordingly, the disclosure provides antibody-drug conjugates comprising the 5T4 antigen binding domains described herein. In some embodiments, the antibody-drug conjugate is biparatopic, i.e. comprises a first antigen binding domain that specifically binds to a first 5T4 epitope, and a second antigen binding domain that binds to a second 5T4 epitope, and the two 5T4 epitopes are not the same. In some embodiments, the antibody-drug conjugate comprises a first antigen binding domain that binds specifically to a first 5T4 epitope, and a second antigen binding domain that specifically binds to a second 5T4 epitope that is not the same as the first 5T4 epitope, and comprises a chemotherapeutic agent.

Biparatopic antibodies, and antibody-drug conjugates thereof of the present disclosure bind two different epitopes of the same target 5T4 molecule, thereby cross-linking 5T4 on the surface of cells such as cancer cells and inducing the formation of 5T4-antibody immunocomplexes. This improved cross-linking provides a functional benefit over known 5T4 binding proteins known in the art, such as monospecific antibodies or other proteins that specifically bind a single 5T4 epitope. Such benefits include a more robust and rapid internalization of the 5T4 molecules bound to the antibody-drug complex, which can lead to better cytotoxic killing of the target cancer cells. Without wishing to be bound by theory, it is thought that increased cross-linking and/or aggregation of antibody immunocomplexes leads to more robust internalization, lysosomal trafficking and degradation of immunocomplexes within cells and intracellular release of cytotoxic payloads within host cells. The biparatopic antibody-drug conjugates described herein show improved internalization and specific killing of 5T4-expressing cancer cells. Thus, the biparatopic antibodies and antibody-drug conjugates of the disclosure can provide improved potency toward cancer cells, and more effective treatment for subjects with 5T4 positive cancers.

In some embodiments, biparatopic antibodies and antibody-drug conjugates of the presented disclosure comprise an IgG constant (Fc) region domain comprising at least one mutation that reduces effector function, extends half-life, or a combination thereof. For example, Fc mutations in the antibody-drug conjugates provided herein provide an added benefit over 5T4 binding proteins known in the prior art by reducing affinity to at least Fc gamma receptor I (FcγRI) and/or Fc gamma receptor IIIa (FcγRIIIa) compared to antibodies without Fc mutations, without diminishing the binding affinity of the antibody-drug conjugates to 5T4. Exemplary Fc mutations that minimize antibody effector function, and extend half-life of the antibody-drug conjugates are shown in Table 3.

In some embodiments, biparatopic antibodies of the disclosure comprise a first antigen binding domain operably linked to a second antigen binding domain. In some embodiments, the first and second antigen binding domains are independently selected from the group consisting of a Fab fragment, a F(ab′)₂ fragment, a scFv, a scab, a dAb, a single domain heavy chain antibody, a single domain light chain antibody, and a full length IgG antibody. In some embodiments, the biparatopic antibody comprises a full-length IgG antibody comprising the first antigen binding domain, and the second antigen binding domain comprises an scFv. In some embodiments, the chemotherapeutic agent is conjugated to at least one of the full-length IgG antibody comprising the first antigen binding domain or the second antigen binding domain via a linker. In some embodiments, the chemotherapeutic agent is an auristatin, such as for example, but not limited to auristatin E (AE), monomethyl auristatin D (MMAD), monomethyl auristatin E (MMAE), monomethyl auristatin F (MMAF), and synthetic analogs of dolastatin.

Also provided herein are polynucleotides and vectors encoding the antigen binding domains, antibodies and antibody-drug conjugates comprising same, and chimeric antigen receptors (CARs) comprising the antigen binding domains of the disclosure, as well as pharmaceutical compositions comprising same, and methods of making and using same. The antigen binding domains, antibodies, antibody-drug conjugates and CARs can be used to treat a variety of diseases and disorders, including cancers.

### Definitions

Unless defined otherwise, technical and scientific terms used herein have the same meaning as commonly understood by one of ordinary skill in the art to which this invention belongs.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

The term “subject” as used herein includes, but is not limited to, a mammal, including, e.g., a human, non-human primate (e.g., monkey), mouse, pig, cow, goat, rabbit, rat, guinea pig, hamster, horse, monkey, sheep, or other non-human mammal, a non-mammal, including, e.g., a non-mammalian vertebrate, such as a bird (e.g., a chicken or duck) or a fish; and a non-mammalian invertebrate. In some embodiments, the methods and compositions of the invention are used to treat (both prophylactically and/or therapeutically) non-human animals. The term “subject” can also refer to patients, i.e., individuals awaiting or receiving medical care.

The term “pharmaceutical composition” herein means a composition suitable for pharmaceutical use in a subject, including an animal or human. A pharmaceutical composition generally comprises an effective amount of an active agent (e.g., the antibodies or antibody-drug conjugates of the disclosure) and a pharmaceutically acceptable carrier, diluent or excipient (e.g., a buffer, adjuvant, or the like).

The term “effective amount” means a dosage or amount sufficient to produce a desired result. The desired result may comprise an objective or subjective improvement in the recipient of the dosage or amount (e.g., long-term survival, decrease in number and/or size of tumors, effective prevention of a disease state, etc.).

A “prophylactic treatment” is a treatment administered to a subject who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder. A “prophylactic activity” is an activity of an agent, such as the anti-5T4 bispecific antibody-drug conjugates of the disclosure, or compositions thereof, that, when administered to a subject who does not display signs or symptoms of a pathology, disease or disorder (or who displays only early signs or symptoms of a pathology, disease, or disorder) diminishes, prevents, or decreases the risk of the subject developing the pathology, disease, or disorder. A “prophylactically useful” agent or compound (e.g., an anti-5T4 bispecific antibody-drug conjugate) refers to an agent or compound that is useful in diminishing, preventing, treating, or decreasing development of a pathology, disease or disorder.

A “therapeutic treatment” is a treatment administered to a subject who displays symptoms or signs of pathology, disease, or disorder, in which treatment is administered to the subject for the purpose of diminishing or eliminating those signs or symptoms of pathology, disease, or disorder. A “therapeutic activity” is an activity of an agent, such an antibody-drug conjugate of the disclosure, or a composition thereof, that eliminates or diminishes signs or symptoms of a pathology, disease or disorder, when administered to a subject suffering from such signs or symptoms. A “therapeutically effective” agent or compound (e.g., an anti-5T4 bispecific antibody-drug conjugate) indicates that an agent or compound is effective in diminishing, treating, or eliminating such signs or symptoms of the pathology, disease or disorder.

The term “treating cancer” as used herein, unless otherwise indicated, means reversing, alleviating, inhibiting the progress of, or preventing, either partially or completely, the growth of tumors, tumor metastases, or other cancer-causing or neoplastic cells in a subject. The term “treatment” as used herein, unless otherwise indicated, refers to the act of treating.

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity equals the number of identical positions/total number of positions (e.g., overlapping positions×100). In some embodiments, the two sequences are the same length.

The term “substantially identical,” in the context of two nucleic acids or polypeptides, refers to two or more sequences or subsequences that have at least 60%, at least 70%, at least 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98% identity, or at least 99% identity (e.g., as determined using one of the methods set forth infra).

The determination of percent identity between two sequences can be accomplished using a mathematical algorithm. A non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to a protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search, which detects distant relationships between molecules (id.). When utilizing BLAST, Gapped BLAST, and PSI-BLAST programs, the default parameters of the respective programs (e.g., XBLAST and NBLAST) can be used. Another non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Nat. Acad. Sci. USA 85:2444-8. Alternatively, protein sequence alignment may be carried out using the CLUSTAL W algorithm, as described by Higgins et al., 1996, Methods Enzymol. 266:383-402.

As used herein, “antigen binding domain” refers to a region on an antibody that binds to antigens. An exemplary antigen binding domain comprises one constant and one variable domain of each of the heavy and light chain of the antibody. However, alternative arrangements, such as, for example, single-domain antigen binding domains, are contemplated as within the scope of the instant disclosure so long as such domains are capable of binding an antigen. Exemplary antigen binding domains include, but are not limited to, scFv, Fab fragments, Fab′ fragments, F(ab′)₂ fragments and the like, and are described in further detail below.

As used herein, the term binds,” “specifically binds to,” or is “specific to” refers to measurable and reproducible interactions such as binding between a target and an antigen binding domain, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antigen binding domain to an unrelated target is less than about 10% of the binding of the antibody to the target as measured, for example, by a radioimmunoassay (RIA). In certain embodiments, an antibody that specifically binds to a target has a dissociation constant (Kd) of <1 μM, <100 nM, <10 nM, <1 nM, <0.1 nM or <0.01 nM.

In certain embodiments, an antigen binding domain specifically binds to an epitope on a protein that is conserved among the protein from different species. In another embodiment, specific binding can include, but does not require exclusive binding.

As used in this specification, the singular forms “a”, “an”, and “the” include plural references unless the context clearly dictates otherwise. Reference to “the formulation” or “the method” includes one or more formulations, methods, and/or steps of the type described herein and/or which will become apparent to those persons skilled in the art upon reading this disclosure.

The term “polypeptide” refers to a polymer of amino acids and its equivalent and does not refer to a specific length of a product; thus, “peptides” and “proteins” are included within the definition of a polypeptide. A protein can have one or more polypeptides. Also included within the definition of polypeptides are “antibodies” as defined herein. A “polypeptide region” refers to a segment of a polypeptide, which segment may contain, for example, one or more domains or motifs (e.g., a polypeptide region of an antibody can contain, for example, one or more complementarity determining regions (CDRs)). The term “fragment” refers to a portion of a polypeptide that is less than the entire polypeptide, as it occurs naturally.

Unless otherwise indicated by context, a “derivative” is a polypeptide or fragment thereof having one or more non-conservative or conservative amino acid substitutions relative to a second polypeptide (also referred to as a “variant”), or deletions or insertions relative thereto; or a polypeptide or fragment thereof that is modified by covalent attachment of a second molecule such as, e.g., by attachment of a heterologous polypeptide, or by glycosylation, acetylation, phosphorylation, and the like. Further included within the definition of “derivative” are, for example, polypeptides containing one or more analogs of an amino acid (e.g., unnatural amino acids and the like), polypeptides with unsubstituted linkages, as well as other modifications known in the art, both naturally and non-naturally occurring.

An “isolated” polypeptide is one which has been identified and separated and/or recovered from a component of its natural environment. Contaminant components of its natural environment are materials which would interfere with diagnostic or therapeutic uses for the polypeptide, and may include enzymes, hormones, and other proteinaceous or nonproteinaceous solutes. An isolated polypeptide includes an isolated antibody, or a fragment or derivative thereof.

As used herein, the term “chimeric antigen receptor (CAR)” refers to an artificial transmembrane protein receptor comprising (i) an extracellular domain capable of binding to at least one predetermined CAR ligand or antigen, such as a 5T4 antigen binding domain as described herein, (ii) an intracellular segment comprising one or more cytoplasmic domains derived from signal transducing proteins different from the polypeptide from which the extracellular domain is derived, and (iii) a transmembrane domain. In some cases, CARs also include a hinge domain. CARs can be used to graft an artificial specificity onto a particular immune effector cell, such as a helper T cell (CD4+), cytotoxic T cell (CD8+) or NK cell. CARs may be employed to impart the specificity of a monoclonal antibody onto a T cell, thereby allowing a large number of specific T cells to be generated, for example, for use in adoptive cell therapy. Depending on the particular architecture of the CAR, and the intracellular signaling domains employed, the CAR may be an activator receptor, or an inhibitory receptor. Exemplary activator CARs comprise a CD3 zeta intracellular domain, one or more intracellular domains for additional co-stimulatory signaling, such as ICOS, CD137 (4-1BB), CD27, CD28, CD134, CD152 (CTLA-4), CD223 (LAG4), DAP10, and/or OX-40, and optionally an extracellular hinge region, for example derived from CD8a or CD28. Many different CARs are known in the art, all of which are envisaged as within the scope if the instant invention.

As used herein, the term “T cell bispecific antibody” refers to an antibody with dual binding specificity for a cancer-associated antigen, such as 5T4, and a CD3 subunit present on the surface of T cells, for example any one of CD3 epsilon, CD3 gamma, CD3 delta, or CD3 zeta. Without wishing to be bound by theory, it is thought that this allows the T cell bispecific antibody to cross link, and bring into proximity, T cells and cancer cells, inducing T cell activation and subsequent cancer cell death.

The term “about” as used herein means in quantitative terms plus or minus 5%, or in another embodiment plus or minus 10%, or in another embodiment plus or minus 15%, or in another embodiment plus or minus 20%.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All publications, patents, and patent applications mentioned in this specification are herein incorporated by reference to the same extent as if each individual publication, patent, or patent application was specifically and individually indicated to be incorporated by reference.

### 5T4

One example of an antigen associated with cancer is the trophoblast glycoprotein (TPBG), also known as human 5T4 antigen (5T4), 5T4 oncofetal antigen, or Wnt-activated inhibitory factor 1 (WAIF1). The human 5T4 antigen is a 72 kDa type I transmembrane glycoprotein expressed in embryonic tissues, such as placenta, and in various types of solid tumors and carcinomas, including prostate cancer, gastric cancer, and colorectal cancer. See, e.g., U.S. Pat. No. 7,074,909 or U.S. Pat. No. 7,514,546. However, the 5T4 antigen is either expressed at low levels or not expressed in most healthy adult epithelial tissues. See Woods et al, Biochem. J. (2002) 366, 353-365.

The expression or overexpression of the 5T4 antigen in various tumor types, particularly in ovarian, gastric and colorectal tumors, is associated with poorer clinical outcomes. Additionally, overexpression is associated with changes in cell morphology and motility that are consistent with tumor invasion. Thus, it is believed that the 5T4 antigen plays a role in the progression or malignancy of some solid tumors.

The disclosure provides antigen binding domains, as well as antibodies, antibody-drug conjugates and receptors comprising antigen binding domains that bind to 5T4.

The disclosure provides antigen binding domains useful for targeting cells, such as cancer cells, that express 5T4 antigen. In some embodiments, the antigen binding domains are incorporated into an antibody-drug conjugate, which can be used for 5T4-targeted cancer therapy. Exemplary antibody-drug conjugates include biparatopic antibodies that bind to two different 5T4 epitopes, as well as 5T4 antibody-drug conjugates that can bind a single 5T4 epitope. In some embodiments, the 5T4 antibody-drug conjugate comprises a biparatopic 5T4 antibody, and binds to two different 5T4 epitopes. The antibody-drug conjugates provided herein can comprise the 5T4 antigen binding portion of an anti-5T4 antibody engineered into a single chain form and fused to a chemotherapeutic agent. In some embodiments, the 5T4 antibody-drug conjugate comprises a biparatopic 5T4 antibody, comprising a full length IgG antibody linked to an scFv.

The disclosure provides pharmaceutical compositions comprising antigen binding domains, antibodies, antibody-drug conjugates, and receptors described herein. In some embodiments, the pharmaceutical compositions comprise a biparatopic 5T4 antibody-drug conjugate comprising a first antigen binding domain that specifically binds to a first 5T4 epitope, a second antigen binding domain that specifically binds to a second 5T4 epitope that is not the same as the first 5T4 epitope, and a chemotherapeutic agent.

Additionally, this disclosure provides methods of treating diseases or disorders involving cellular expression of the 5T4 antigen, the methods comprising administering to a subject in need of thereof a therapeutically effective amount of a pharmaceutical composition described herein, for example a pharmaceutical composition comprising a biparatopic 5T4 antibody-drug conjugate comprising a first antigen binding domain that specifically binds to a first 5T4 epitope, a second antigen binding domain that specifically binds to a second 5T4 epitope that is not the same as the first 5T4 epitope, and a chemotherapeutic agent.

The present disclosure provides antigen binding domains that bind to human 5T4 antigen. Human 5T4 comprises an amino acid sequence according to the/NCBI Reference Sequence NP_001363851.1:

```
(SEQ ID NO: 27)
    1 MPGGCSRGPA AGDGRLRLAR LALVLLGWVS SSSPTSSASS FSSSAPFLAS AVSAQPPLPD
  61 QCPALCECSE AARTVKCVNR NLTEVPTDLP AYVRNLFLTG NQLAVLPAGA FARRPPLAEL
121 AALNLSGSRL DEVRAGAFEH LPSLRQLDLS HNPLADLSPF AFSGSNASVS APSPLVELIL
181 NHIVPPEDER QNRSFEGMVV AALLAGRALQ GLRRLELASN HFLYLPRDVL AQLPSLRHLD
241 LSNNSLVSLT YVSFRNLTHL ESLHLEDNAL KVLHNGTLAE LQGLPHIRVF LDNNPWVCDC
301 HMADMVTWLK ETEVVQGKDR LTCAYPEKMR NRVLLELNSA DLDCDPILPP SLQTSYVFLG
361 IVLALIGAIF LLVLYLNRKG IKKWMHNIRD ACRDHMEGYH YRYEINADPR LTNLSSNSDV.
```

Any suitable antigen binding domain capable of specifically binding to 5T4, or a fragment thereof, is envisaged within the scope of the instant disclosure, including, but not limited to, a Fab fragment, a F(ab′)2 fragment, single chain variable fragments (scFv), a scab, a dAb, single domain antibodies (sdAb) such as VHH single domain antibodies, a single domain heavy chain antibody, or single domain light chain antibody, full length IgG antibodies, antibody fragments, antigen binding domains, or fragments comprising antigen binding domains as described in further detail below. In addition, full length antibodies, biparatopic antibodies, T cell bispecific antibodies, fusion proteins, and receptors such as chimeric antigen receptors described herein are contemplated as within the scope of the instant disclosure.

The disclosure provides antibodies comprising the antigen binding domains specific to the 5T4 antigen described herein. For example, the antibodies can be monoclonal antibodies, such as full length IgG antibodies.

As used herein, an “antibody” refers to a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains, respectively. The term “antibody” includes antibody molecules prepared, expressed, created or isolated by recombinant means, such as antibodies isolated from a host cell transfected to express the antibody. The term antibody also includes bispecific antibodies (e.g., T cell bispecific antibodies), or biparatopic antibodies, which can include a heterotetrameric immunoglobulin that can bind to more than one different epitope. Bispecific antibodies are generally described in US Patent Application Publication No. 2010/0331527, which is incorporated by reference into this application. The term antibody herein is used in the broadest sense and specifically covers intact monoclonal antibodies, polyclonal antibodies, multispecific antibodies (e.g. bispecific antibodies) formed from at least two intact antibodies, and antibody fragments, so long as they exhibit the desired biological activity.

The term ‘antibody” also includes one or more fragments of an antibody that retain the ability to specifically bind to an antigen, such as for example, an antibody-binding portion or domain of an antibody, sometimes referred to herein as an “antigen binding domain” or “antigen binding portion.”

The term “antigen binding domain” or “antigen binding region” as used herein refers to a domain of an antigen binding moiety that is responsible for the specific binding between an antigen binding moiety and an antigen. For example, the antigen binding region of an antibody or a fragment thereof is formed by amino acid residues of the N-terminal variable regions of the heavy chain (abbreviated herein as VH) and the light chain (abbreviated herein as VL). The variable regions of the VH and the VL each comprise three hypervariable regions, termed complementary determining regions (CDR). The 3 CDRs of the VH and the 3 CDRs of the VL are three-dimensionally disposed relative to each other to form an antigen binding surface. Examples of binding fragments encompassed within the term “antigen binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 241:544-546), which consists of a VH domain, (vi) an isolated CDR, and (vii) an scFv, which consists of the two domains of the Fv fragment, VL and VH, joined by a synthetic linker to form a single protein chain in which the VL and VH regions pair to form monovalent molecules. Other forms of single chain antibodies, such as diabodies are also encompassed under the term “antibody” (see e.g., Holliger et al. (1993) PNAS USA 90:6444-6448; Poljak et al. (1994) Structure 2:1 121-1 123).

Still further, an antibody or antigen binding domain thereof may be part of a larger immunoadhesion molecule, formed by covalent or noncovalent association of the antibody or antigen binding domain with one or more other proteins or peptides. Examples of such immunoadhesion molecules include use of the streptavidin core region to make a tetrameric scFv molecule (Kipriyanov et al. (1995) Human Antibodies and Hybridomas 6:93-101) and use of a cysteine residue, a marker peptide and a C-terminal polyhistidine tag to make bivalent and biotinylated scFv molecules (Kipriyanov et al. (1994) Mol. Immunol. 31: 1047-1058). Antibody fragments, such as Fab and F(ab′)2 fragments, can be prepared from whole antibodies using conventional techniques, such as via papain or pepsin digestion of whole antibodies. Moreover, antibodies, antibody fragments and immunoadhesion molecules can be obtained using standard recombinant DNA techniques commonly known in the art (see Sambrook et al., 1989).

The term “human antibody” or “humanized antibody” is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. “Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity, and capacity. In some instances, Fv framework residues of the human immunoglobulin are replaced by corresponding non-human residues. Furthermore, humanized antibodies may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. The general structure of antibodies is known in the art. Briefly, an immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus, once a set of CDR sequences (i.e. the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

Antibodies, or antigen binding domains, exist as intact immunoglobulins or as a number of well characterized fragments produced by digestion with various peptidases. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the F(ab′)2dimer into an Fab′ monomer. The Fab′ monomer is essentially a Fab with part of the hinge region. While various antibody fragments are defined in terms of the digestion of an intact antibody, one of skill will appreciate that such Fab′ fragments, etc. may be synthesized de novo either chemically or by utilizing recombinant DNA methodology. Thus, the term antibody, as used herein also includes antibody fragments either produced by the modification of whole antibodies or synthesized de novo using recombinant DNA methodologies.

Antibodies, or antigen binding domains, include single chain antibodies, for example single chain Fv (sFv or scFv) antibodies in which a variable heavy and a variable light chain are joined together (directly or through a peptide linker) to form a continuous polypeptide.

Antibodies, or antigen binding domains, include single domain antibodies, which comprise an antibody fragment consisting of a single monomeric variable antibody domain that is able to bind selectively to an antigen domain. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine.

As explained above, the antibodies or antigen binding domains used herein optionally comprise F(ab)2, F(ab′)2, Fab, Fab′, scFv, single domain antibodies, etc. depending upon the specific requirements of the embodiment. Some embodiments use antibodies comprising IgG domains. However, other embodiments comprise alternate immunoglobulins such as IgM, IgA, IgD, and IgE. Furthermore, all possible isotypes of the various immunoglobulins are also encompassed within the current embodiments. Thus, IgG1, IgG2, IgG3, IgG4 etc. are all possible molecules in the antibody domains used in the invention. In addition to choice in selection of the type of immunoglobulin and isotype, different embodiments of the invention may comprise various hinge regions (or functional equivalents thereof). Such hinge regions provide flexibility between the different domains of the antibody, and e.g., an effector to which the antibody is fused.

Antibodies or antigen binding domains of the disclosure include “chimeric” antibodies or antigen binding domains (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

In some embodiments, the antibodies and antibody-drug conjugates of the instant disclosure comprise an antigen binding domain specific to 5T4 comprising any of the CDRs disclosed in Tables 1-2.

TABLE 1
Heavy Chain CDR sequences for 5T4 binding domains

| No. | CDRH1 | CDRH2 | CDRH3 |
|---|---|---|---|
| 1 | GYSFTDYYMH | RVSPNNGATNTNQKFK | STMITSYYFDY |
| | (SEQ ID NO: 1) | D (SEQ ID NO: 2) | (SEQ ID NO: 3) |
| 2 | GFDFSRYWMT | EINPDSRTINYTPSLKD | PDHDYNPYYFNY |
| | (SEQ ID NO: 4) | (SEQ ID NO: 5) | (SEQ ID NO: 6) |
| 3 | GFDFSRYWMT | EINPDSNTINYTPSLKD | PDYDYNPYYFAY |
| | (SEQ ID NO: 4) | (SEQ ID NO: 24) | (SEQ ID NO: 40) |
| 4 | GFTFSSFGLH | YISVGSSTIYYADPVKG | SRTYYRSEIDS |
| | (SEQ ID NO: 13) | (SEQ ID NO: 25) | (SEQ ID NO: 41) |
| 5 | GFNIKDTYMH | RIDPADGNTKFDPKFQ | FRRYDYVMDY |
| | (SEQ ID NO: 14) | G (SEQ ID NO: 26) | (SEQ ID NO: 42) |
| 6 | GYSFTGYYLH | RVNPNNGATVYNQNF | SIMITTYDFDY |
| | (SEQ ID NO: 15) | KG (SEQ ID NO: 28) | (SEQ ID NO: 43) |
| 7 | GYSFTGYYLH | RVNPNNGGTIYNQNFK | SIMITTFDFDY |
| | (SEQ ID NO: 15) | G (SEQ ID NO: 29) | (SEQ ID NO: 44) |
| 8 | GYTFSSYWIE | EILPGSGRTNYNEKFKG | GNYGSSPYYFDY |
| | (SEQ ID NO: 16) | (SEQ ID NO: 30) | (SEQ ID NO: 45) |
| 9 | GYTFTHYVIS | EIYPGSGSTYYNEKFKG | GGRYGFDY (SEQ |
| | (SEQ ID NO: 17) | (SEQ ID NO: 31) | ID NO: 46) |
| 10 | GFSLSTSGVGVD | HIWWDDVKRYNPALK | IADGYSAPWFAY |
| | (SEQ ID NO: 18) | S (SEQ ID NO: 32) | (SEQ ID NO: 47) |
| 11 | GFTFSNFGMH | YISSGTSTIYYADTVKG | SRAYYRYEIDH |
| | (SEQ ID NO: 19) | (SEQ ID NO: 33) | (SEQ ID NO: 48) |
| 12 | GFTFSSFGMH | YISSGGSNIYYADTVKG | WGDSYRYFDV |
| | (SEQ ID NO: 20) | (SEQ ID NO: 34) | (SEQ ID NO: 49) |
| 13 | GFDFSRYWMT | EINPDSRTINYTPPLKD | PDHEYNPYYFNN |
| | (SEQ ID NO: 4) | (SEQ ID NO: 35) | (SEQ ID NO: 50) |
| 14 | GFDFSRYWMT | EINPDSRTINYTPSLKD | PDHDYNPYYFNY |
| | (SEQ ID NO: 4) | (SEQ ID NO: 5) | (SEQ ID NO: 6) |
| 15 | GFDFSRYWMT | EINPDSRTINYTPSLKD | PDHDYNPYYFNY |
| | (SEQ ID NO: 4) | (SEQ ID NO: 5) | (SEQ ID NO: 6) |
| 16 | GFNIKDTYMH | RIDPANGNTKYDPKFQ | FRRYALVMDY |
| | (SEQ ID NO: 14) | G (SEQ ID NO: 36) | (SEQ ID NO: 51) |
| 17 | GYSFTGYYLH | RVNPNNGGTSYNQKFK | STMITSYYFDY |
| | (SEQ ID NO: 15) | G (SEQ ID NO: 37) | (SEQ ID NO: 3) |
| 18 | GYSFTGYYIH | RVNPNNGGTIYNQKFK | STMITSYYFDY |
| | (SEQ ID NO: 21) | G (SEQ ID NO: 38) | (SEQ ID NO: 3) |
| 19 | GYTFSTYWIE | EILPGSGRNNYNEKFK | GNYGSSPYYFDY |
| | (SEQ ID NO: 22) | G (SEQ ID NO: 39) | (SEQ ID NO: 45) |
| 20 | GFSLSTSGMGVG | HIWWDDVKRYNPALK | IADGYYAPWFAY |
| | (SEQ ID NO: 23) | S (SEQ ID NO: 32) | (SEQ ID NO: 52) |

TABLE 2
Light Chain CDR sequences for 5T4 binding domains

| No. | CDRL1 | CDRL2 | CDRL3 |
|---|---|---|---|
| 1 | KASQSVSTDVA | FASDRYT | QQDYSSPPT |
| | (SEQ ID NO: 7) | (SEQ ID NO: 8) | (SEQ ID NO: 9) |
| 2 | RPSQSVSTSSNSYI | YASNLES | QQSWEIPLT |
| | H | (SEQ ID NO: 11) | (SEQ ID NO: 12) |
| | (SEQ ID NO: 10) | | |
| 3 | RASQSVSTSRYSY | YASNLES (SEQ | QHSWEIPLT (SEQ ID |
| | MH (SEQ ID NO: | ID NO: 11) | NO: 76) |
| | 53) | | |
| 4 | RASQSVSSSSYNY | SASTLES (SEQ ID | QHSWEIPYT (SEQ ID |
| | MH (SEQ ID NO: | NO: 67) | NO: 77) |
| | 54) | | |
| 5 | RASESVDSYGNIF | LASNLES (SEQ ID | QQNNEDPWT (SEQ ID |
| | MH (SEQ ID NO: | NO: 68) | NO: 78) |
| | 55) | | |
| 6 | KASQDVSIDVG | WASTRHT (SEQ | QQHYSTPPT (SEQ ID |
| | (SEQ ID NO: 56) | ID NO: 69) | NO: 79) |
| 7 | KASQDVNTAVV | WASTRHT (SEQ | QQHYSTPPT (SEQ ID |
| | (SEQ ID NO: 57) | ID NO: 69) | NO: 79) |
| 8 | RASQSVSTSRNSY | YASNLES (SEQ | QHSWEIPLT (SEQ ID |
| | MH (SEQ ID NO: | ID NO: 11) | NO: 76) |
| | 58) | | |
| 9 | TASSSVSSSYLH | STSNLAS (SEQ ID | HQYHRSPLT (SEQ ID |
| | (SEQ ID NO: 59) | NO: 70) | NO: 80) |
| 10 | KASQDINKYIA | YTSTLQP (SEQ ID | LQCDNLWT (SEQ ID |
| | (SEQ ID NO: 60) | NO: 71) | NO: 81) |
| 11 | RASQSVSTSSSNS | NASNLES (SEQ | QHSWEIPYT (SEQ ID |
| | YVH (SEQ ID NO: | ID NO: 72) | NO: 77) |
| | 61) | | |
| 12 | SASSSVSFMH | STSNLAS (SEQ ID | QQRSGYPPKFT (SEQ |
| | (SEQ ID NO: 62) | NO: 70) | ID NO: 82) |
| 13 | RPSQSVSTSSNSYI | YASNLEP (SEQ | QQSWEIPLT (SEQ ID |
| | H (SEQ ID NO: 10) | ID NO: 73) | NO: 12) |
| 14 | RPSQSVSTSSNSYI | YASNLES (SEQ | QQSWEIPLT (SEQ ID |
| | H (SEQ ID NO: 10) | ID NO: 11) | NO: 12) |
| 15 | RASQSVSTSRYSY | YASNLES (SEQ | QHSWEIPLT (SEQ ID |
| | IH (SEQ ID NO: 63) | ID NO: 11) | NO: 76) |
| 16 | RASETIDSYGNTF | LASNLES (SEQ ID | QQNNEDPWT (SEQ ID |
| | MH (SEQ ID NO: | NO: 68) | NO: 78) |
| | 64) | | |
| 17 | KASQSVSNDVA | YVSNRYI (SEQ ID | QQDYSSPPT (SEQ ID |
| | (SEQ ID NO: 65) | NO: 74) | NO: 9) |
| 18 | KASQSVNYDVA | YASKRYT (SEQ | QQDYSSPPT (SEQ ID |
| | (SEQ ID NO: 66) | ID NO: 75) | NO: 9) |
| 19 | RASQSVSTSRNSY | YASNLES (SEQ | QHSWEIPLT (SEQ ID |
| | MH (SEQ ID NO: | ID NO: 11) | NO: 76) |
| | 58) | | |
| 20 | KASQDINKYIA | YTSTLQP (SEQ ID | LQCDDLWT (SEQ ID |
| | (SEQ ID NO: 60) | NO: 71) | NO: 83) |

The person of ordinary skill in the art will understand that each row in Table 1 discloses three heavy chain CDR sequences and each row in Table 2 discloses three light chain CDR sequences, which together can bind to 5T4. Thus, antigen binding domains specific to 5T4 disclosed herein will be understood to have, in some embodiments, six CDRs, three for the variable heavy domain and three for the variable light domain, corresponding to rows at the same positions in Tables 1 and 2, respectively, identified by the numbers at left. Any combination of CDRs comprising a row of CDRs from Table 1, combined with a row of CDRs from Table 2, is envisaged as within the scope of the 5T4 antibodies of the disclosure. In addition, antibodies, antigen binding domains, CDRs and sequences thereof to that specifically bind to 5T4 epitopes can be derived by methods known in the art. For example, the monoclonal antibodies to be used herein may be made by the hybridoma method first described by Kohler et al., 1975, Nature 256:495, or may be made by recombinant DNA methods (see, for example, U.S. Pat. No. 4,816,567). Monoclonal antibodies may also be isolated from phage antibody libraries using the techniques described in Clackson et al., 1991, Nature 352:624-628 and Marks et al., 1991, J. Mol. Biol. 222:581-597, for example, and the sequences of the antibodies, and corresponding encoding nucleic acids determined by methods known in the art.

Bispecific antibodies are antibodies that have binding specificities for at least two different antigens. Biparatopic antibodies are antibodies that bind to two distinct epitopes of the same antigen. Both bispecific antibodies, e.g., antibodies that bind to 5T4 using a 5T4 antigen binding domain as described herein, and that also bind to an additional antigen, as well as biparatopic antibodies that bind to 5T4, are contemplated as within the scope of the instant disclosure.

The present disclosure provides biparatopic antibody-drug conjugates having a first antigen binding domain that binds to a first 5T4 epitope, and a second antigen binding domain that binds to a second 5T4 epitope that is not the same as the first 5T4 epitope, wherein the first antigen binding domain is operably linked to the second antigen binding domain. In some embodiments, the first antigen binding domain is linked to the second antigen binding domain via linker.

Antibodies with more than two valencies are also contemplated as within the scope of the disclosure. For example, trispecific antibodies can be prepared. See Tutt et al., J. Immunol. 147:60 (1991).

In some aspects, the first and second antigen binding domains are independently selected from the group consisting of a Fab fragment, a F(ab′)2 fragment, a scFv, a scab, a dAb, a single domain heavy chain antibody, a single domain light chain antibody, and a full length IgG antibody.

In some aspects, the biparatopic antibodies of the disclosure comprise a first antibody comprises a full-length IgG antibody comprising a first antigen binding domain. In some aspects, the second antigen binding domain comprises an scFv. In some aspects, the antibody-drug conjugate is tetravalent for binding a 5T4 antigen.

In some aspects, the 5T4 biparatopic antibodies described herein comprise an scFv. In some embodiments, the antibody-drug conjugate comprises two scFv that both specifically bind a 5T4 epitope. In some aspects, the scFv comprises a heavy chain and a light chain, wherein the C-terminus of the light chain is operably linked to the N-terminus of the heavy chain via a linker, or the C-terminus of the heavy chain is operably linked to the N-terminus of the light chain via a linker. In some embodiments, the linker comprises a sequence of SEQ ID NO: 153.

In some aspects, the biparatopic antibodies comprise an scFv and a full length IgG antibody. In some embodiments, the N-terminus of the scFv is operably linked to the C-terminus of a heavy chain of the full length IgG antibody. In some aspects, the C-terminus of the ScFv is operably linked to the N-terminus of a heavy chain of the full length IgG antibody.

In some aspects, the scFv is operably linked to the heavy chain of the full length IgG antibody using a linker. In some aspects, the linker comprises or consists of an amino acid sequence of SEQ ID NO: 152.

In some aspects of the biparatopic antibody-drug conjugates described herein, the biparatopic antibody comprises a full-length IgG antibody comprising the first antigen binding domain, and the second antigen binding domain comprises an scFv, and the antibody-drug conjugate comprises four polypeptides comprising two polypeptides comprising, from N to C terminus, the full length IgG antibody heavy chain, a linker, and the second antigen binding domain; and two polypeptides comprising the full length IgG antibody light chain.

In some aspects of the antibody-drug conjugates, the antibody-drug conjugate comprises a full-length IgG antibody comprising the first antigen binding domain and the second antigen binding domain comprises an scFv, and the antibody-drug conjugate comprises four polypeptides comprising two polypeptides comprising, from N to C terminus, the second antigen binding domain, a linker, and the full length IgG antibody heavy chain; and two polypeptides comprising the full length IgG antibody light chain.

As used herein, “binding affinity” refers to the tendency of one molecule to bind (typically non-covalently) with another molecule, such as the tendency of a member of a specific binding pair for another member of a specific binding pair. A binding affinity can be measured as a dissociation constant, which for a specific binding pair (such as an antibody/antigen pair) can be lower than 1×10⁻⁵ M, lower than 1×10⁻⁶ M, lower than 1×10⁻⁷ M, lower than 1×10⁻⁸ M, lower than 1×10⁻⁹ M, lower than 1×10⁻¹⁰ M, lower than 1×10⁻¹¹ M or lower than 1×10⁻¹² M. In one aspect, binding affinity is calculated by a modification of the Scatchard method described by Frankel et al., Mol. Immunol., 16:101-106, 1979. In another aspect, binding affinity is measured by a binding constant. In another aspect, binding affinity is measured by an antigen/antibody dissociation rate. In yet another aspect, a high binding affinity is measured by a competition radioimmunoassay.

The term “dissociation constant”, or “KD” (M), as used herein, refers to the dissociation equilibrium constant of a particular antibody-antigen interaction. In one aspect, the KD is determined by for instance surface plasmon resonance (SPR) technology in a BIAcore 8000 instrument using the antigen as the ligand and the antibody as the analyte. In some aspects, the antibody binds to the predetermined antigen with an affinity corresponding to a KD that is at least ten-fold lower, such as at least 100-fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the KD of the antibody, so that when the KD of the antibody is very low (that is, the antibody is highly specific), then the amount with which the affinity for the antigen is lower than the affinity for a non-specific antigen may be at least 10,000-fold.

In some aspects, the equilibrium dissociation constant (KD) of the antigen binding domain for binding 5T4 is between about 1×10⁻¹² and about 1×10⁻⁷ M, about 1×10⁻¹² and about 1×10⁻⁸ M, about 1×10⁻¹² and about 1×10⁻⁹ M, about 1×10⁻¹¹ and about 1×10⁻⁹ M, or about 1×10⁻¹¹ and about 9×10⁻¹⁰ M. In some aspects, the KD of the antigen binding domain for binding 5T4 is between about 7.42×10⁻¹¹ and about 7.75×10⁻¹⁰ M. In some aspects, the KD is less than or equal to 7.75×10⁻¹⁰ M, e.g. less than or equal to 3.20×10⁻¹⁰ M, less than or equal to 1.98×10⁻¹⁰ M, or less than or equal to 7.42×10⁻¹¹ M. In some aspects, the KD for binding 5T4 is between about 3.63×10⁻¹² and about 1.43×10⁻⁹ M. In some aspects, the KD for binding 5T4 is between about 3.63×10⁻¹² and about 1.34×10⁻⁹ M. In some aspects, the KD of the antigen binding domain for binding 5T4 is between about 3.63×10⁻¹² and about 1.59×10⁻¹¹ M. In some aspects, the KD for binding 5T4 is between about 7.42×10⁻¹¹ and about 7.75×10⁻¹⁰ M. In some aspects, the KD for binding 5T4 is between about 7.42×10⁻¹¹ and about 7.75×10⁻¹⁰ M.

In some aspects of the biparatopic antibodies or biparatopic antibody-drug conjugates described herein, the equilibrium dissociation constant (KD) for binding 5T4 of at least one of the first antigen binding domain or the second antigen binding domain is between about 1×10⁻¹² and about 1×10⁻⁷ M, about 1×10⁻¹² and about 1×10⁻⁸ M, about 1×10⁻¹² and about 1×10⁻⁹ M, about 1×10⁻¹¹ and about 1×10⁻⁹ M, or about 1×10⁻¹¹ and about 9×10⁻¹⁰ M. In some aspects, the KD for binding 5T4 of at least one of the first antigen binding domain or the second antigen binding domain is between about 7.42×10⁻¹¹ and about 7.75×10⁻¹⁰ M. In some aspects, the KD for binding 5T4 of at least one of the first antigen binding domain or the second antigen binding domain is between about 3.63×10⁻¹² and about 7.75×10⁻¹⁰ M. In some aspects, the KD is less than or equal to 7.75×10⁻¹⁰ M, e.g. less than or equal to 3.20×10⁻¹⁰ M, less than or equal to 1.98×10⁻¹⁰ M, or less than or equal to 7.42×10⁻¹¹ M. In some aspects, the KD for binding 5T4 of the first antibody or antigen binding domain is between about 7.42×10⁻¹¹ and about 7.75×10⁻¹⁰ M. In some aspects, the KD of the first antibody or antigen binding domain for binding 5T4 is between about 3.63×10⁻¹² and about 1.43×10⁻⁹ M. In some aspects, the KD of the first antibody or antigen binding domain is between about 3.63×10⁻¹² and about 1.34×10⁻⁹ M. In some aspects, the KD of the first antibody or antigen binding domain is between about 3.63×10⁻¹² and about 1.59×10⁻¹¹ M. In some aspects, the KD for binding 5T4 of the at least second antibody or antigen binding domain is between about 7.42×10⁻¹¹ and about 7.75×10⁻¹⁰ M. In some aspects, the KD of the second antibody or antigen binding domain for binding 5T4 is between about 3.63×10⁻¹² and about 1.43×10⁻⁹ M. In some aspects, the KD of the second antibody or antigen binding domain is between about 3.63×10⁻¹² and about 1.34×10⁻⁹ M. In some aspects, the KD of the second antibody or antigen binding domain is between about 3.63×10⁻¹² and about 1.59×10⁻¹¹ M. In some aspects, the KD for binding 5T4 of both the first antibody or antigen binding domain and the at least second antibody or antigen binding domain is between about 7.42×10⁻¹¹ and about 7.75×10⁻¹⁰ M. In some aspects, the KD is between about 3.63×10⁻¹² and about 7.75×10⁻¹⁰ M. In some aspects, the KD is between about 3.63×10⁻¹² and about 1.59×10⁻¹¹ M.

In some aspects, the equilibrium dissociation constant (KD) for binding Fc gamma receptor I of an antibody comprising the antigen binding domain is less than or equal to about 1.0×10⁻⁷ M, e.g. less than or equal to about 5.0×10⁻⁸ M, less than or equal to about 4.0×10⁻⁸ M, less than or equal to about 3.0×10⁻⁸ M, less than or equal to about 1.0×10⁻⁸ M, less than or equal to about 5.0×10⁻⁹ M or less than or equal to about 1.0×10⁻⁹ M. In some aspects, the KD is less than or equal to about 3.96×10⁻⁸ M. In some aspects, the KD is less than 3.73×10⁻⁹ M.

In some aspects, the KD for binding Fc gamma receptor IIa of an antibody comprising the antigen binding domain is less than or equal to about 3.74×10⁻⁶ M, e.g. less than or equal to about 5.0×10⁻⁷ M, less than or equal to about 1.0×10⁻⁷ M, less than or equal to about 9.86×10⁻⁸ M, less than or equal to about 9.15×10⁻⁸ M, less than or equal to about 5.0×10⁻⁸ M or less than or equal to about 1.0×10⁻⁸ M. In some aspects, the KD for binding Fc gamma receptor IIa of the antibody comprising the antigen binding domain is less than or equal to about 9.15×10⁻⁸ M. In some aspects, the KD for binding Fc gamma receptor IIa is less than or equal to about 9.86×10⁻⁸ M.

In some aspects, the KD for binding Fc gamma receptor IIb of an antibody comprising the antigen binding domain is less than or equal to about 2.0×10⁻⁷ M, e.g. less than or equal to about 1.16×10⁻⁷ M, less than or equal to about 1.11×10⁻⁷ M, less than or equal to about 1.0×10⁻⁷ M, less than or equal to about 5.0×10⁻⁸ M, less than or equal to about 1.0×10⁻⁸ M or less than or equal to about 5.0×10⁻⁹ M. In some aspects, the KD for binding Fc gamma receptor IIb is less than or equal to about 1.16×10⁻⁷ M. In some aspects, the KD for binding Fc gamma receptor IIb is less than or equal to about 1.11×10⁻⁷ M.

In some aspects, the KD for binding Fc gamma receptor IIIa of an antibody comprising the antigen binding domain is less than or equal to about 6.0×10⁻⁷ M, e.g. less than or equal to about 1.0×10⁻⁷ M, less than or equal to about 7.0×10⁻⁸ M, less than or equal to about 5.05×10⁻⁸ M, less than or equal to about 5.0×10⁻⁸ M, less than or equal to about 1.0×10⁻⁸ M, or less than or equal to about 5.0×10⁻⁹ M. In some aspects, the KD for binding Fc gamma receptor IIIa is less than or equal to about 6.0×10⁻⁸ M. In some aspects, the KD for binding Fc gamma receptor IIIa is less than or equal to about 5.05×10⁻⁸ M. In some aspects, the antibody does not detectably bind the Fc gamma receptor IIIa.

In some aspects, the antibody comprising the antigen binding domain does not detectably bind the Fc gamma receptor IIIb.

In some aspects, the equilibrium dissociation constant (KD) for binding Fc gamma receptor I of an antibody comprising at least one of the first and/or second antigen binding domain is less than or equal to about 1.0×10⁻⁷ M, e.g. less than or equal to about 5.0×10⁻⁸ M, less than or equal to about 4.0×10⁻⁸ M, less than or equal to about 3.0×10⁻⁸ M, less than or equal to about 1.0×10⁻⁸ M, less than or equal to about 5.0×10⁻⁹ M or less than or equal to about 1.0×10⁻⁹ M. in some aspects, the KD is less than or equal to about 3.96×10⁻⁸ M. In some aspects, the KD is less than 3.73×10⁻⁹ M.

In some aspects, the KD for binding Fc gamma receptor IIa of an antibody comprising at least one of the first and/or second antigen binding domain is less than or equal to about 3.74×10⁻⁶ M, e.g. less than or equal to about 5.0×10⁻⁷ M, less than or equal to about 1.0×10⁻⁷ M, less than or equal to about 9.86×10⁻⁸ M, less than or equal to about 9.15×10⁻⁸ M, less than or equal to about 5.0×10⁻⁸ M or less than or equal to about 1.0×10⁻⁸ M. In some aspects, the KD for binding Fc gamma receptor IIa is less than or equal to about 9.15×10⁻⁸ M. In some aspects, the KD for binding Fc gamma receptor IIa is less than or equal to about 9.86×10⁻⁸ M. In some aspects, the KD for binding Fc gamma receptor IIb is less than or equal to about 1.16×10⁻⁷ M.

In some aspects, the KD for binding Fc gamma receptor IIb of an antibody comprising at least one of the first and/or second antigen binding domain is less than or equal to about 2.0×10⁻⁷ M, e.g. less than or equal to about 1.16×10⁻⁷ M, less than or equal to about 1.11×10⁻⁷ M, less than or equal to about 1.0×10⁻⁷ M, less than or equal to about 5.0×10⁻⁸ M, less than or equal to about 1.0×10⁻⁸ M or less than or equal to about 5.0×10⁻⁹ M. In some aspects, the KD for binding Fc gamma receptor IIb is less than or equal to about 1.16×10⁻⁷ M. In some aspects, the KD for binding Fc gamma receptor IIb is less than or equal to about 1.11×10⁻⁷ M.

In some aspects, the KD for binding Fc gamma receptor IIIa of an antibody comprising at least one of the first and/or second antigen binding domain is less than or equal to about 6.0×10⁻⁷ M, e.g. less than or equal to about 1.0×10⁻⁷ M, less than or equal to about 7.0×10⁻⁸ M, less than or equal to about 5.05×10⁻⁸ M, less than or equal to about 5.0×10⁻⁸ M, less than or equal to about 1.0×10⁻⁸ M, or less than or equal to about 5.0×10⁻⁹ M. In some aspects, the KD for binding Fe gamma receptor 1IIa is less than or equal to about 6.0×10⁻⁸ M. In some aspects, the KD for binding Fc gamma receptor 1IIa is less than or equal to about 5.05×10⁻⁸ M. In some aspects, the antibody does not detectably bind the Fc gamma receptor IIIa.

In some aspects, the antibody comprising at least one of the first and/or second antigen binding domain does not detectably bind the Fc gamma receptor IIb.

In some aspects, the first and second antibody bind different 5T4 molecules on the surface of different cancer cells. In some aspects, the first and second antibody bind different 5T4 molecules on the surface of the same cancer cell. In some aspects, the first antibody and second antibody bind the same 5T4 molecule on the surface of a cancer cell. In some aspects, the first and second 5T4 epitopes are non-overlapping epitopes.

### Fc Effector Function

In some embodiments, site mutations in the Fc region of the antibody-drug conjugates mitigate potential side effects caused by antibody effector function, for example at least binding affinity to Fc gamma receptors (FcγRs). Without wishing to be bound by theory, it is thought that binding to Fc gamma receptors can induce activating or inhibitory pathways of the immune system, such as for example antibody-dependent cell-mediated cytotoxicity (ADCC), antibody-dependent cellular phagocytosis (ADCP), and complement-dependent cytotoxicity (CDC).

In some embodiments, the antibody-drug conjugates described herein comprise a full-length IgG antibody. In some embodiments, the full-length IgG antibody comprises a constant region (Fc) domain. In some embodiments, for example those embodiments where the antibody-drug conjugate comprises an IgG Fc domain, the Fc domain is an IgG1 isotype constant region domain. In some embodiments, the IgG1 constant region domain comprises at least one mutation that reduces effector function, extends half-life, or a combination thereof. In some embodiments, the antibody-drug conjugates disclosed herein comprise Fc mutations that reduce binding affinity to Fc gamma receptors. In some embodiments, the Fc mutations reduce binding affinity to an Fc gamma receptor that includes FcγRI. In some embodiments, the Fc mutations reduce binding affinity to an Fc gamma receptor that includes FcγRII. In some embodiments, the Fc mutations reduce binding affinity to an Fc gamma receptor that includes FcγRIII. In some embodiments, the Fc mutation of the antibody-drug conjugate reduce the binding affinity toward FcγRI compared to a 5T4 antibody without Fc mutations. In some embodiments, the at least one mutation comprises an F at position 237 relative to SEQ ID NO: 100 (L234F), a C or A at position 242 relative to SEQ ID NO: 100 (S239C/A), or a combination thereof.

In some embodiments, the Fc site mutations that reduce binding affinity toward FcγRI comprise L234F and S239C, or S239A.

In some embodiments, the Fc domain can be an IgG1 isotype constant region domain. In some embodiments, the IgG1 constant region domain comprises at least one mutation that reduces effector function, extends half-life, or a combination thereof. In some embodiments, the IgG1 constant region domain mutations extend the half-life of the antibody-drug conjugate. Fc mutations of the antibody-drug conjugate can extend the half-life of the antibody-drug conjugate. In some embodiments, the at least one mutation comprises a mutation at position 437 relative to SEQ ID NO: 100 (N434). In some embodiments, the at least one mutation, wherein the mutation comprises an A at position 437 relative to SEQ ID NO: 100 (N434A). These amino acids are indicated with bold and underlining in Table 5, below. In some embodiments, the antibody-drug conjugate comprises at least one mutation that extends the half-life of the antibody-drug conjugate, wherein the at least one mutation comprises a mutation at position 437, wherein the mutation further comprises an A at position 437 relative to SEQ ID NO: 100 (N434A). In some embodiments, the antibody-drug conjugate comprises a mutation that extends the half-life of the antibody-drug conjugate, wherein the mutation comprises a mutation at position 437, wherein the mutation further comprises an A at position 437 relative to SEQ ID NO: 100 (N434A). In some embodiments, the antibody-drug conjugate comprises one mutation that extends the half-life of the antibody-drug conjugate, wherein the one mutation comprises a mutation at position 437, wherein the mutation further comprises an A at position 437 relative to SEQ ID NO: 100 (N434A).

TABLE 3
Summary of Equilibrium Dissociation Constants
(KD) for binding of Exemplary Antibody-drug
conjugates to select Fc gamma receptors (FcγRs)

| | |
|---|---|
| Fc | KD |

| Receptor | 5T4-Bs3- FCA | Parental Ab1 without Fc mutations |
|---|---|---|
| FcγRI | 3.96E−08 | 3.73E−09 |
| FcγRIIa | 9.15E−08 | 9.86E−08 |
| FcγRIIb | 1.16E−07 | 1.11E−07 |
| FcγRIIIa | No binding | 5.05E−08 |
| FcγRIIIb | No binding | No binding |

In some aspects, the antibody-drug conjugates disclosed herein comprise a monoclonal antibodies. In some aspects, the monoclonal antibody binds only a single 5T4 epitope. In some aspects, the monoclonal antibody comprises only a first antibody, i.e. is not a bispecific antibody. In some aspects, the monoclonal antibody binds only a single epitope, i.e., is not a biparatopic antibody.

In some aspects, the antibodies disclosed herein are bispecific or biparatopic monoclonal antibodies. In some aspects, the antibodies comprise a first antigen binding domain, for example an scFv, and a second antigen binding domain, for example a full length IgG antibody. In some aspects, the antibodies comprise a first antigen binding domain that binds to a first 5T4 epitope, and a second antigen binding domain that binds to a second 5T4 epitope.

In some aspects, the antibodies comprise a first antigen binding domain that binds to a 5T4 epitope, and a second antigen binding domain that binds to a second antigen, for example an antigen expressed by cancer cells. Suitable cancer antigens, and antigen binding domains that bind to these antigens, will be known by persons known in the art, and include, for example, B-cell maturation antigen (BCMA), CD19 molecule (CD19), CD20, CD30, CD33, CD38, CD44, CD123, CD138, cell adhesion molecule (CEA), C-type lectin domain family 12 member A (CLEC12A), chorionic somatomammotropin hormone 1 (CS-1), epidermal growth factor receptor (EGFR), EGFRvIII, epithelial cell adhesion molecule (EPCAM), delta like canonical Notch ligand 3 (DLL3), leucine rich repeat containing G protein-coupled receptor 5 (LGR5), mesothelin (MSLN), Programmed death-ligand 1 (PD-L1), folate receptor alpha (FOLR1), folate receptor gamma (FOLR3), erb-b2 receptor tyrosine kinase 2 (ERBB2 or HER2), HER3, bone marrow stromal cell antigen 2 (HM1.24), Sperm Mitochondria Associated Cysteine Rich Protein (MCSP), and Prostate-specific membrane antigen (PSMA). Alternatively, the bispecific antibodies disclosed herein are T cell bispecific (TCB) antibodies. In these cases, the second antigen is expressed by T cells, e.g. a CD3e antigen, and the bispecific antibodies described herein help recruit and engage T cells in the presence of 5T4 positive cancer cells.

In some embodiments, the antigen binding domains of the present disclosure comprise an antigen binding domain specific to 5T4 comprising any of the CDRs disclosed in Tables 1-2. Exemplary variable heavy and light chains of the antigen binding domains of the disclosure incorporating the CDRs disclosed in Tables 1-2 are provided in Table 4, below.

TABLE 4
Anti-5T4 Heavy and Light Chain Sequences

| | Amino Acid | |
|---|---|---|
| Ab | Sequence | Nucleotide Sequence |
| VH-1 | QVQLVQSGAEVKKPGA | CAGGTTCAGCTGGTACAGAGCGGAGCTGAAGTCAAGAA |
| (Variable | SVKVSCKASGYSFTDY | ACCTGGGGCTTCAGTGAAGGTTAGCTGCAAAGCCTCTG |
| Heavy) | YMHWVRQAPGQGLEWM | GCTACAGCTTCACAGACTACTACATGCACTGGGTTAGA |
| | GRVSPNNGATNTNQKF | CAGGCACCAGGACAAGGGCTTGAGTGGATGGGTCGAGT |
| | KDRVTMTRDTSISTAY | GTCTCCCAACAATGGAGCCACCAATACCAACCAGAAGT |
| | MELRSLRSEDTAVYYC | TCAAGGATCGTGTCACCATGACTCGGGATACCTCCATT |
| | ARSTMITSYYFDYWGQ | TCCACTGCCTATATGGAACTGCGCTCCTTGAGGAGTGA |
| | GTLVTVSS (SEQ ID | GGACACAGCAGTGTACTATTGCGCTAGGAGTACCATGA |
| | NO: 96) | TAACGTCCTATTACTTCGACTATTGGGGCCAAGGTACT |
| | | CTGGTGACAGTGTCTAGC (SEQ ID NO: 144) |
| VH-2 | EVQLLESGGGLVQPGG | GAGGTGCAGCTGCTGGAATCTGGCGGAGGATTGGTTCA |
| | SLRLSCAASGFDFSRY | GCCTGGCGGCTCTCTGAGACTGTCTTGTGCCGCCTCTG |
| | WMTWVRQAPGKCLEWV | GCTTCGACTTCTCCCGGTATTGGATGACCTGGGTCCGA |
| | SEINPDSRTINYTPSL | CAGGCTCCTGGCAAGTGTCTGGAATGGGTGTCCGAGAT |
| | KDRFTISRDNAKNTLY | CAACCCCGACAGCCGGACCATCAACTACACCCCTAGCC |
| | LQMNSLRAEDTAVYYC | TGAAGGACCGGTTCACCATCTCCAGAGACAACGCCAAG |
| | ARPDHDYNPYYFNYWG | AACACCCTGTACCTGCAGATGAACTCCCTGAGAGCCGA |
| | QGTTVTVSS (SEQ | GGACACCGCCGTGTACTACTGTGCCAGACCTGACCACG |
| | ID NO: 97) | ACTACAACCCCTACTACTTCAACTACTGGGGCCAGGGC |
| | | ACCACCGTGACAGTTTCTAGC (SEQ ID NO: 145) |
| VL-1 | DIVMTQSPSFLSVSVG | GACATCGTGATGACACAGTCACCCTCCTTTCTGAGTGT |
| | DRVTITCKASQSVSTD | GAGTGTTGGGGATAGAGTCACCATCACATGCAAAGCCA |
| | VAWYQQKPGQAPKLLI | GCCAATCAGTCAGCACCGATGTAGCCTGGTATCAGCAG |
| | YFASDRYTGVPDRFSG | AAACCAGGCCAAGCTCCCAAGCTGCTGATCTACTTCGC |
| | SGSGTDFTFTISSLQA | AAGCGACAGGTACACTGGTGTGCCAGATCGGTTTTCTG |
| | EDVAVYYCQQDYSSPP | GGTCTGGATCTGGTACTGACTTCACCTTCACGATCTCC |
| | TFGGGTKLEIK (SEQ | AGTCTGCAGGCTGAAGATGTTGCCGTCTACTACTGTCA |
| | ID NO: 98) | GCAGGACTATTCCTCTCCTCCTACCTTTGGAGGAGGCA |
| | | CTAAGCTGGAGATAAAGC (SEQ ID NO: 146) |
| VL-2 | DIVLTQSPASLAVSLG | GATATCGTGCTGACCCAGTCTCCTGCCAGCCTGGCTGT |
| | QRATISCRPSQSVSTS | TTCTCTGGGACAGAGAGCCACCATCAGCTGCCGGCCTT |
| | SNSYIHWYQQKPGQPP | CTCAGTCCGTGTCCACCTCCTCCAACTCCTACATCCAC |
| | KLLIKYASNLESGVPA | TGGTATCAGCAGAAGCCCGGCCAGCCTCCTAAGCTGCT |
| | RFSGSGSGTDFTLTIS | GATTAAGTACGCCTCCAACCTGGAATCCGGCGTGCCAG |
| | PVEAEDTATYYCQQSW | CCAGATTTTCCGGCTCTGGCTCTGGCACCGACTTCACC |
| | EIPLTFGCGTKLEIK | CTGACAATCTCTCCCGTGGAAGCTGAGGATACCGCCAC |
| | (SEQ ID NO: 99) | CTACTACTGCCAGCAGTCCTGGGAGATCCCTCTGACCT |
| | | TTGGCTGTGGCACCAAGCTGGAAATCAAA (SEQ ID |
| | | NO: 147) |
| HC | ASTKGPSVFPLAPSSK | GCGTCGACAAAGGGCCCCTCCGTGTTTCCTCTGGCTCC |
| constant | STSGGTAALGCLVKDY | AAGCTCTAAGAGCACCTCTGGAGGAACAGCCGCTCTGG |
| | FPEPVTVSWNSGALTS | GATGTCTGGTGAAGGATTACTTCCCTGAGCCAGTGACC |
| | GVHTFPAVLQSSGLYS | GTGAGCTGGAACTCTGGCGCCCTGACCTCCGGAGTGCA |
| | LSSVVTVPSSSLGTQT | TACATTTCCCGCTGTGCTGCAGTCCAGCGGCCTGTATA |
| | YICNVNHKPSNTKVDK | GCCTGTCTTCCGTGGTGACCGTGCCTAGCTCTTCCCTG |
| | RVEPKSCDKTHTCPPC | GGCACCCAGACATACATCTGCAACGTGAATCACAAGCC |
| | PAPEFLGGPCVFLFPP | CTCCAATACAAAGGTGGACAAGAGAGTGGAGCCTAAGA |
| | KPKDTLMISRTPEVTC | GCTGTGATAAGACCCATACATGCCCACCATGTCCAGCT |
| | VVVDVSHEDPEVKFNW | CCTGAGTTCCTGGGAGGACCTTGCGTGTTCCTGTTTCC |
| | YVDGVEVHNAKTKPRE | TCCAAAGCCAAAGGACACCCTGATGATCTCTCGCACCC |
| | EQYNSTYRVVSVLTVL | CTGAGGTGACATGCGTGGTGGTGGACGTGTCCCACGAG |
| | HQDWLNGKEYKCKVSN | GATCCAGAGGTGAAGTTCAACTGGTACGTGGATGGCGT |
| | KALPAPIEKTISKAKG | GGAGGTGCATAATGCTAAGACCAAGCCTAGGGAGGAGC |
| | QPREPQVYTLPPSREE | AGTACAACAGCACCTATCGGGTGGTGTCTGTGCTGACA |
| | MTKNQVSLTCLVKGFY | GTGCTGCACCAGGACTGGCTGAACGGCAAGGAGTACAA |
| | PSDIAVEWESNGQPEN | GTGCAAGGTGAGCAATAAGGCCCTGCCAGCTCCCATCG |
| | NYKTTPPVLDSDGSFF | AGAAGACCATCTCTAAGGCCAAGGGCCAGCCCAGAGAG |
| | LYSKLTVDKSRWQQGN | CCTCAGGTGTATACACTGCCCCCTAGCCGCGAGGAGAT |
| | VFSCSVMHEALHAHYT | GACCAAGAACCAGGTGTCTCTGACCTGTCTGGTGAAGG |
| | QKSLSLSPGK (SEQ | GCTTCTACCCATCTGACATCGCTGTGGAGTGGGAGTCC |
| | ID NO: 148) | AATGGCCAGCCCGAGAACAATTATAAGACCACACCACC |
| | | CGTGCTGGACTCCGATGGCAGCTTCTTTCTGTACTCCA |
| | | AGCTGACCGTGGATAAGAGCAGGTGGCAGCAGGGCAAC |
| | | GTGTTTTCCTGCAGCGTGATGCACGAGGCCCTGCACGC |
| | | TCATTATACACAGAAATCTCTGTCCCTGAGCCCAGGCA |
| | | AG (SEQ ID NO: 150) |
| LC | RTVAAPSVFIFPPSDE | GTACGGTGGCCGCTCCATCCGTGTTCATCTTTCCACCC |
| constant | QLKSGTASVVCLLNNF | TCCGACGAGCAGCTGAAGTCCGGAACCGCTAGCGTGGT |
| | YPREAKVQWKVDNALQ | GTGCCTGCTGAACAACTTCTACCCAAGAGAGGCCAAGG |
| | SGNSQESVTEQDSKDS | TGCAGTGGAAGGTGGATAACGCTCTGCAGAGCGGCAAT |
| | TYSLSSTLTLSKADYE | TCTCAGGAGTCCGTGACCGAGCAGGACAGCAAGGATTC |
| | KHKVYACEVTHQGLSS | TACATATTCCCTGAGCTCTACCCTGACACTGTCCAAGG |
| | PVTKSFNRGEC (SEQ | CCGATTACGAGAAGCACAAGGTGTATGCTTGCGAGGTG |
| | ID NO: 149) | ACCCATCAGGGCCTGTCCAGCCCCGTGACAAAGAGCTT |
| | | CAACCGCGGCGAGTGT (SEQ ID NO: 151) |
| Linker-1 | GGGGSGGGGSGGGGS | GGTGGAGGGGGATCcGGCGGCGGCGGCAGTGGCGGAGG |
| | (SEQ ID NO: 152) | AGGCTCC (SEQ ID NO: 154) |
| Linker-2 | GGGGSGGGGSGGGGSG | GGCGGAGGTGGAAGCGGAGGCGGAGGTAGTGGTGGTGG |
| | GGGS (SEQ ID NO: | CGGATCTGGTGGCGGTGGATCT (SEQ ID NO: 155) |
| | 153) | |

Exemplary full length heavy and light chains of the antigen binding domains of the disclosure are provided in Table 5, below (HC: heavy chain; LC: light chain). In Table 5, the full sequence heavy chain of the first antibody, a full length IgG1 antibody, is italicized, with mutations to in the constant region underlined. The first linker is underlined, and the VH and VL sequences of the second antibody, an scFv, are in bold and joined by a linker which is italicized and underlined.

TABLE 5
Anti-5T4 Heavy and Light Chain Full Length Amino Acid Sequences

| Ab | Amino Acid Sequence | Nucleotide Sequence |
|---|---|---|
| HC1 | QVQLVQSGAEVKKPGASVKV | CAGGTTCAGCTGGTACAGAGCGGAGCTGAAGTCAAGAA |
| Heavy | SCKASGYSFTDYYMHWVRQA | ACCTGGGGCTTCAGTGAAGGTTAGCTGCAAAGCCTCTG |
| Chain | PGQGLEWMGRVSPNNGATNT | GCTACAGCTTCACAGACTACTACATGCACTGGGTTAGA |
| Full | NQKFKDRVTMTRDTSISTAY | CAGGCACCAGGACAAGGGCTTGAGTGGATGGGTCGAGT |
| length | MELRSLRSEDTAVYYCARST | GTCTCCCAACAATGGAGCCACCAATACCAACCAGAAGT |
| | MITSYYFDYWGQGTLVTVSS | TCAAGGATCGTGTCACCATGACTCGGGATACCTCCATT |
| | ASTKGPSVFPLAPSSKSTSG | TCCACTGCCTATATGGAACTGCGCTCCTTGAGGAGTGA |
| | GTAALGCLVKDYFPEPVTVS | GGACACAGCAGTGTACTATTGCGCTAGGAGTACCATGA |
| | WNSGALTSGVHTFPAVLQSS | TAACGTCCTATTACTTCGACTATTGGGGCCAAGGTACT |
| | GLYSLSSVVTVPSSSLGTQT | CTGGTGACAGTGTCTAGCGCGTCGACAAAGGGCCCCTC |
| | YICNVNHKPSNTKVDKRVEP | CGTGTTTCCTCTGGCTCCAAGCTCTAAGAGCACCTCTG |
| | KSCDKTHTCPPCPAPEFLGG | GAGGAACAGCCGCTCTGGGATGTCTGGTGAAGGATTAC |
| | PCVFLFPPKPKDTLMISRTP | TTCCCTGAGCCAGTGACCGTGAGCTGGAACTCTGGCGC |
| | EVTCVVVDVSHEDPEVKFNW | CCTGACCTCCGGAGTGCATACATTTCCCGCTGTGCTGC |
| | YVDGVEVHNAKTKPREEQYN | AGTCCAGCGGCCTGTATAGCCTGTCTTCCGTGGTGACC |
| | STYRVVSVLTVLHQDWLNGK | GTGCCTAGCTCTTCCCTGGGCACCCAGACATACATCTG |
| | EYKCKVSNKALPAPIEKTIS | CAACGTGAATCACAAGCCCTCCAATACAAAGGTGGACA |
| | KAKGQPREPQVYTLPPSREE | AGAGAGTGGAGCCTAAGAGCTGTGATAAGACCCATACA |
| | MTKNQVSLTCLVKGFYPSDI | TGCCCACCATGTCCAGCTCCTGAGTTCCTGGGAGGACC |
| | AVEWESNGQPENNYKTTPPV | TTGCGTGTTCCTGTTTCCTCCAAAGCCAAAGGACACCC |
| | LDSDGSFFLYSKLTVDKSRW | TGATGATCTCTCGCACCCCTGAGGTGACATGCGTGGTG |
| | QQGNVFSCSVMHEALHAHYT | GTGGACGTGTCCCACGAGGATCCAGAGGTGAAGTTCAA |
| | QKSLSLSPGKGGGGSGGGGS | CTGGTACGTGGATGGCGTGGAGGTGCATAATGCTAAGA |
| | GGGGSEVQLLESGGGLVQPG | CCAAGCCTAGGGAGGAGCAGTACAACAGCACCTATCGG |
| | GSLRLSCAASGFDFSRYWMT | GTGGTGTCTGTGCTGACAGTGCTGCACCAGGACTGGCT |
| | WVRQAPGKCLEWVSEINPDS | GAACGGCAAGGAGTACAAGTGCAAGGTGAGCAATAAGG |
| | RTINYTPSLKDRFTISRDNA | CCCTGCCAGCTCCCATCGAGAAGACCATCTCTAAGGCC |
| | KNTLYLQMNSLRAEDTAVYY | AAGGGCCAGCCCAGAGAGCCTCAGGTGTATACACTGCC |
| | CARPDHDYNPYYFNYWGQGT | CCCTAGCCGCGAGGAGATGACCAAGAACCAGGTGTCTC |
| | TVTVSSGGGGSGGGGSGGGG | TGACCTGTCTGGTGAAGGGCTTCTACCCATCTGACATC |
| | SGGGGSDIVLTQSPASLAVS | GCTGTGGAGTGGGAGTCCAATGGCCAGCCCGAGAACAA |
| | LGQRATISCRPSQSVSTSSN | TTATAAGACCACACCACCCGTGCTGGACTCCGATGGCA |
| | SYIHWYQQKPGQPPKLLIKY | GCTTCTTTCTGTACTCCAAGCTGACCGTGGATAAGAGC |
| | ASNLESGVPARFSGSGSGTD | AGGTGGCAGCAGGGCAACGTGTTTTCCTGCAGCGTGAT |
| | FTLTISPVEAEDTATYYCQQ | GCACGAGGCCCTGCACGCTCATTATACACAGAAATCTC |
| | SWEIPLTFGCGTKLEIK | TGTCCCTGAGCCCAGGCAAGGGTGGAGGGGGATCcGGC |
| | (SEQ ID NO: 100) | GGCGGCGGCAGTGGCGGAGGAGGCTCCGAGGTGCAGCT |
| | | GCTGGAATCTGGCGGAGGATTGGTTCAGCCTGGCGGCT |
| | | CTCTGAGACTGTCTTGTGCCGCCTCTGGCTTCGACTTC |
| | | TCCCGGTATTGGATGACCTGGGTCCGACAGGCTCCTGG |
| | | CAAGTGTCTGGAATGGGTGTCCGAGATCAACCCCGACA |
| | | GCCGGACCATCAACTACACCCCTAGCCTGAAGGACCGG |
| | | TTCACCATCTCCAGAGACAACGCCAAGAACACCCTGTA |
| | | CCTGCAGATGAACTCCCTGAGAGCCGAGGACACCGCCG |
| | | TGTACTACTGTGCCAGACCTGACCACGACTACAACCCC |
| | | TACTACTTCAACTACTGGGGCCAGGGCACCACCGTGAC |
| | | AGTTTCTAGCGGCGGAGGTGGAAGCGGAGGCGGAGGTA |
| | | GTGGTGGTGGCGGATCTGGTGGCGGTGGATCTGATATC |
| | | GTGCTGACCCAGTCTCCTGCCAGCCTGGCTGTTTCTCT |
| | | GGGACAGAGAGCCACCATCAGCTGCCGGCCTTCTCAGT |
| | | CCGTGTCCACCTCCTCCAACTCCTACATCCACTGGTAT |
| | | CAGCAGAAGCCCGGCCAGCCTCCTAAGCTGATTAAGTA |
| | | CGCCTCCAACCTGGAATCCGGCGTGCCAGCCAGATTTT |
| | | CCGGCTCTGGCTCTGGCACCGACTTCACCCTGACAATC |
| | | TCTCCCGTGGAAGCTGAGGATACCGCCACCTACTACTG |
| | | CCAGCAGTCCTGGGAGATCCCTCTGACCTTTGGCTGTG |
| | | GCACCAAGCTGGAAATCAAA (SEQ ID NO: 142) |
| LC-1 | DIVMTQSPSFLSVSVGDRVT | GACATCGTGATGACACAGTCACCCTCCTTTCTGAGTGT |
| Light | ITCKASQSVSTDVAWYQQKP | GAGTGTTGGGGATAGAGTCACCATCACATGCAAAGCCA |
| Chain | GQAPKLLIYFASDRYTGVPD | GCCAATCAGTCAGCACCGATGTAGCCTGGTATCAGCAG |
| Full | RFSGSGSGTDFTFTISSLQA | AAACCAGGCCAAGCTCCCAAGCTGCTGATCTACTTCGC |
| Length | EDVAVYYCQQDYSSPPTFGG | AAGCGACAGGTACACTGGTGTGCCAGATCGGTTTTCTG |
| | GTKLEIKRTVAAPSVFIFPP | GGTCTGGATCTGGTACTGACTTCACCTTCACGATCTCC |
| | SDEQLKSGTASVVCLLNNFY | AGTCTGCAGGCTGAAGATGTTGCCGTCTACTACTGTCA |
| | PREAKVQWKVDNALQSGNSQ | GCAGGACTATTCCTCTCCTCCTACCTTTGGAGGAGGCA |
| | ESVTEQDSKDSTYSLSSTLT | CTAAGCTGGAGATAAAGCGTACGGTGGCCGCTCCATCC |
| | LSKADYEKHKVYACEVTHQG | GTGTTCATCTTTCCACCCTCCGACGAGCAGCTGAAGTC |
| | LSSPVTKSFNRGEC (SEQ | CGGAACCGCTAGCGTGGTGTGCCTGCTGAACAACTTCT |
| | ID NO: 101) | ACCCAAGAGAGGCCAAGGTGCAGTGGAAGGTGGATAAC |
| | | GCTCTGCAGAGCGGCAATTCTCAGGAGTCCGTGACCGA |
| | | GCAGGACAGCAAGGATTCTACATATTCCCTGAGCTCTA |
| | | CCCTGACACTGTCCAAGGCCGATTACGAGAAGCACAAG |
| | | GTGTATGCTTGCGAGGTGACCCATCAGGGCCTGTCCAG |
| | | CCCCGTGACAAAGAGCTTCAACCGCGGCGAGTGT |
| | | (SEQ ID NO: 143) |

Tables 6 and 7 provide nucleotide sequences encoding exemplary CDRs for use in the 5T4 antigen binding domains of the disclosure.

TABLE 6
Exemplary Anti-5T4 Heavy Chain CDR
Nucleotide Sequences

| | CDRH1 | CDRH2 | CDRH3 |
|---|---|---|---|
| A | GGCTACAGCTT | CGAGTGTCTCCCAA | AGTACCATGATAACGTC |
| | CACAGACTACT | CAATGGAGCCACCA | CTATTACTTCGACTATT |
| | ACATGCACT | ATACCAACCAGAAG | (SEQ ID NO: 86) |
| | (SEQ ID NO: | TTCAAGGAT (SEQ | |
| | 84) | ID NO: 85) | |
| B | GGCTTCGACTT | GAGATCAACCCCGA | TGACCACGACTACAACC |
| | CTCCCGGTATT | CAGCCGGACCATCA | CCTACTACTTCAACTAC |
| | GGATGACC | ACTACACCCCTAGC | (SEQ ID NO: 89) |
| | (SEQ ID NO: | CTGAAGGAC (SEQ | |
| | 87) | ID NO: 88) | |

TABLE 7
Exemplary Anti-5T4 Light Chain CDRs
Nucleotide Sequences

| | CDRH1 | CDRH2 | CDRH3 |
|---|---|---|---|
| C | GCCAGCCAATCAGT | GCAAGCGACAG | CAGCAGGACTATTCC |
| | CAGCACCGATGTAG | GTACACT (SEQ | TCTCCTCCTACC |
| | CC (SEQ ID NO: | ID NO: 91) | (SEQ ID NO: 92) |
| | 90) | | |
| D | CGGCCTTCTCAGTC | TACGCCTCCAA | CAGCAGTCCTGGGAG |
| | CGTGTCCACCTCCT | CCTGGAATCC | ATCCCTCTGACC |
| | CCAACTCCTACATC | (SEQ ID NO: | (SEQ ID NO: 95) |
| | CAC (SEQ ID NO: | 94) | |
| | 93) | | |

Table 8 provides additional humanized variable heavy and light chain amino acid sequences for the 5T4 antigen binding domains of the disclosure. In Table 8, CDR sequences are in bold.

TABLE 8
Anti-5T4 Humanized Variable Heavy and Variable
Light Chain Amino Acid Sequences

| ID | | | SEQ ID |
|---|---|---|---|
| No. | Domain | Amino Acid Sequence | NO |
| 2 | 5T4-VH | EVQLLESGGGLVQPGGSLRLSCAASGFDFSRYWMTWVR | 102 |
| | | QAPGKGLEWVSEINPDSRTINYTPSLKDRFTISRDNAK | |
| | | NTLYLQMNSLRAEDTAVYYCARPDHDYNPYYFNYWGQG | |
| | | TTVTVSS | |
| | 5T4-VL | DIVLTQSPASLAVSLGQRATISCRPSQSVSTSSNSYIH | 103 |
| | | WYQQKPGQPPKLLIKYASNLESGVPARFSGSGSGTDFT | |
| | | LTISPVEAEDTATYYCQQSWEIPLTFGGGTKLEIK | |
| 3 | 5T4-VH | EVQLLESGGGLVQPGGSLRLSCAASGFDFSRYWMTWVR | 104 |
| | | QAPGKGLEWVSEINPDSNTINYTPSLKDRFTISRDNAK | |
| | | NTLYLQMNSLRAEDTAVYYCASPDYDYNPYYFAYWGQG | |
| | | TLVTVSS | |
| | 5T4-VL | DIVLTQSPASLAVSLGQRATISCRASQSVSTSRYSYMH | 105 |
| | | WYQQKPGQPPKLLIKYASNLESGVPARFSGSGSGTDFT | |
| | | LTISSLQAEDFATYYCQHSWEIPLTFGGGTKLEIK | |
| 4 | 5T4-VH | EVOLVESGGGLVQPGGSLRLSCAASGFTFSSFGLHWVR | 106 |
| | | QAPGKGLEWVAYISVGSSTIYYADPVKGRFTISRDNAK | |
| | | NTLYLQMNSLRAEDTAVYYCARSRTYYRSEIDSWGQGT | |
| | | TVTVSS | |
| | 5T4-VL | DIVLTQSPASLAVSLGQRATISCRASQSVSSSSYNYMH | 107 |
| | | WYQQKPGQPPKLLIYSASTLESGVPARFSGSGSGTDFT | |
| | | LTISSLQAEDFATYYCQHSWEIPYTFGQGTKLEIK | |
| 5 | 5T4-VH | EVQLVQSGAEVKKPGASVKVSCKASGFNIKDTYMHWVR | 108 |
| | | QAPGQGLEWMGRIDPADGNTKFDPKFQGRVTITADTST | |
| | | STAYMELSSLRSEDTAVYYCARFRRYDYVMDYWGOGTT | |
| | | VTVSS | |
| | 5T4-VL | DIVLTQSPASLAVSLGQRATITCRASESVDSYGNIFMH | 109 |
| | | WYQQKPGQPPKLLIYLASNLESGVPARFSGSGSGTDFT | |
| | | LTISSLEAEDFATYYCQQNNEDPWTFGGGTKLEIK | |
| 1 | 5T4-VH | QVQLVQSGAEVKKPGASVKVSCKASGYSFTDYYMHWVR | 96 |
| | | QAPGQGLEWMGRVSPNNGATNTNQKFKDRVTMTRDTSI | |
| | | STAYMELRSLRSEDTAVYYCARSTMITSYYFDYWGQGT | |
| | | LVTVSS | |
| | 5T4-VL | DIVMTQSPSFLSVSVGDRVTITCKASQSVSTDVAWYQQ | 98 |
| | | KPGQAPKLLIYFASDRYTGVPDRFSGSGSGTDFTFTIS | |
| | | SLQAEDVAVYYCQODYSSPPTFGGGTKLEIK | |
| 6 | 5T4-VH | EVQLVQSGAEVKKPGASVKVSCKASGYSFTGYYLHWVR | 112 |
| | | QAPGKGLEWMGRVNPNNGATVYNQNFKGRVTMTRDKSI | |
| | | STAYMELRSLRSEDTAVYYCVRSIMITTYDFDYWGQGT | |
| | | LVTVSS | |
| | 5T4-VL | DIVMTQSPSSLSASVGDRVTITCKASQDVSIDVGWYQQ | 113 |
| | | KPGQAPKLLIYWASTRHTGVPDRFSGSGSGTDFTLTIS | |
| | | SLOPEDFATYYCQQHHIVPPTFGQGTKVEIK | |
| 7 | 5T4-VH | EVQLVQSGAEVKKPGASVKVSCKASGYSFTGYYLHWVR | 114 |
| | | QAPGQCLEWMGRVNPNNGGTIYNONFKGRVTMTRDKSI | |
| | | STAYMELRSLRSEDTAVYYCARSIMITTFDFDYWGQGT | |
| | | LVTVSS | |
| | 5T4-VL | DIVMTQSPSSLSASVGDRVTITCKASQDVNTAVVWYQQ | 115 |
| | | KPGQAPELLIYWASTRHTGVPDRFSGSGSGTDYTLTIS | |
| | | SLQAEDFATYYCQQHYSTPPTFGQGTKVEIK | |
| 8 | 5T4-VH | QVQLVQSGAEVKKPGASVKISCKASGYTFSSYWIEWVR | 116 |
| | | QAPGQGLEWMGEILPGSGRTNYNEKFKGRVTITADTST | |
| | | STAYMELSSLRSEDTAVYYCARGNYGSSPYYFDYWGQG | |
| | | TLVTVSS | |
| | 5T4-VL | DIVLTQSPASLAVSLGQRATISCRASQSVSTSRNSYMH | 117 |
| | | WYQQKPGQPPKLLIKYASNLESGVPARFSGSGSGTDFT | |
| | | LTISSLEPEDFATYYCQHSWEIPLTFGGGTKLEIK | |
| 9 | 5T4-VH | QVQLVQSGAEVKKPGASVKVSCKASGYTFTHYVISWVR | 118 |
| | | QATGQGLEWIGEIYPGSGSTYYNEKFKGRVTMTADKSI | |
| | | STAYMELSSLRSEDTAVYYCARGGRYGFDYWGQGTTVT | |
| | | VSS | |
| | 5T4-VL | EIVLTQSPATLSASPGERVTLTCTASSSVSSSYLHWYQ | 119 |
| | | QKPGLAPKLLIYSTSNLASGVPARFSGSGSGTDYTLTI | |
| | | SSLEPEDFATYYCHQYHRSPLTFGGGTKLEIK | |
| 10 | 5T4-VH | QVTLKESGPTLVKPTQTLTLTCTFSGFSLSTSGVGVDW | 120 |
| | | IRQPPGKALEWLAHIWWDDVKRYNPALKSRLTISKDTS | |
| | | KNQVVLTMTNMDPVDTATYYCARIADGYSAPWFAYWGQ | |
| | | GTLVTVSS | |
| | 5T4-VL | DIQMTQSPSSLSASVGDRVTITCKASQDINKYIAWYQQ | 121 |
| | | KPGKAPKLLIYYTSTLQPGIPSRFSGSGSGTDFTFTIS | |
| | | SLQPEDIATYYCLQCDNLWTFGGGTKLEIK | |

The person of ordinary skill in the art will appreciate that a 5T4 antigen binding domain may comprise any combination of variable heavy and variable light domains disclosed in Table 8 or 9. Alternatively, 5T4 antigen binding domains may comprise the specific pair of variable heavy and variable light domains identified by the number in the left column of Table 8 or 9.

Table 9 provides additional mouse variable heavy and light chain amino acid sequences for the 5T4 antigen binding domains of the disclosure. The person of ordinary skill in the art will understand that the mouse variable heavy and light domains can be humanized.

TABLE 9
Anti-5T4 mouse Variable Heavy and Variable Light
Chain Amino Acid Sequences

| ID | | | SEQ ID |
|---|---|---|---|
| Ab | Domain | Amino Acid Sequence | NO |
| 11 | 5T4_VH | DVQLVESGGGLVQPGGSRKLSCAASGFTFSNFGMHWVR | 122 |
| | | QAPEKGLEWVAYISSGTSTIYYADTVKGRFTISRDNPK | |
| | | NTLFLQMTSLRSEDTAMYYCARSRAYYRYEIDHWGQGT | |
| | | TLTVSS | |
| | 5T4_VL | DIVLTQSPASLAVSLGQRATISCRASQSVSTSSSNSYV | 123 |
| | | HWYQQKLGQPPKLLIKNASNLESGVPARFSGSGSGTDF | |
| | | ILNIHPVEEEDTATYYCQHSWEIPYTFGGGTKLEIK | |
| 12 | 5T4_VH | DVQLVESGGGLAQPGGSRKLSCEASGFTFSSFGMHWVR | 124 |
| | | QPPEKGLEWVAYISSGGSNIYYADTVKGRFTISRDNPK | |
| | | NTLSLQMTSLRSEDTAMYYCARWGDSYRYFDVWGAGTT | |
| | | VTVSS | |
| | 5T4_VL | QIVLTQSPAILSASPGEKVSITCSASSSVSFMHWFQQK | 125 |
| | | PGTSPKLWIYSTSNLASGVPARFSGSGSGTSYSLTISR | |
| | | MEAEDAATYYCQQRSGYPPKFTFGSGTKLEIK | |
| 13 | 5T4_VH | EVKLLESGGGLVQPGGSLKLSCAASGFDFSRYWMTWVR | 126 |
| | | QAPGKGLEWIGEINPDSRTINYTPPLKDKIIISRDNAK | |
| | | NTLYLQMNKVRSEDTALYYCARPDHEYNPYYFNNWGQG | |
| | | TTLTVSS | |
| | 5T4_VL | DIVLTQSPASLPVSLGQRATISCRPSQSVSTSSNSYIH | 127 |
| | | WYQQKPGQPPKLLIKYASNLEPGVPARFSGSGSGTDFT | |
| | | LNIHPVEEEDTATYYCQQSWEIPLTFGAGTKLELK | |
| 2, 14 | 5T4_VH | EVKLLESGGGLVQPGGSLKLSCAASGFDFSRYWMTWVR | 128 |
| | | QAPGKGLEWIGEINPDSRTINYTPSLKDKFIISRDNAK | |
| | | NTLYLHMSKVRSEDTALYYCARPDHDYNPYYFNYWGQG | |
| | | TTLTVSS | |
| | 5T4_VL | DIVLTQSPASLAVSLGQRATISCRPSQSVSTSSNSYIH | 129 |
| | | WYQQKPGQPPKLLIKYASNLESGVPARFSGSGSGTDFT | |
| | | LNIHPVEEEDTATYYCQQSWEIPLTFGAGTKLELK | |
| 15 | 5T4_VH | EVKLLESGGGLVQPGGSLKLSCAASGFDFSRYWMTWVR | 128 |
| | | QAPGKGLEWIGEINPDSRTINYTPSLKDKFIISRDNAK | |
| | | NTLYLHMSKVRSEDTALYYCARPDHDYNPYYFNYWGQG | |
| | | TTLTVSS | |
| | 5T4_VL | DIVVTQSPASLAVSLGQRATISCRASQSVSTSRYSYIH | 131 |
| | | WYQQKPGQSPKLLIKYASNLESGVPPRFSGSGSGTDFT | |
| | | LNIHPVEEEDTATYYCQHSWEIPLTFGAGTKLELK | |
| 16 | 5T4_VH | EVQLQQSGAELVKPGASVKLSCTASGFNIKDTYMHWVK | 132 |
| | | QRPEQGLEWIGRIDPANGNTKYDPKFQGKATITSDTSS | |
| | | NTAYLQLSSLTSEDTAVYYCARFRRYALVMDYWGQGTS | |
| | | VTVSS | |
| | 5T4_VL_ | NIVLTQSPASLAVSLGQRATISCRASETIDSYGNTFMH | 133 |
| | | WYHQKPGQPPKLLIYLASNLESGVPARFSGSGSRTDFT | |
| | | LTIDPVEADDAATYYCQQNNEDPWTFGGGTKLEIK | |
| 17 | 5T4_VH_ | EVQLQQSGPDLVKPGASVRISCKASGYSFTGYYLHWVK | 134 |
| | | QSHGESLEWIGRVNPNNGGTSYNQKFKGKAILTVDTSS | |
| | | NTVYMELRSLTSEDSAVYNCARSTMITSYYFDYWGQGT | |
| | | TLTVSS | |
| | 5T4_VL_ | SIVMTQTPKFLLVSSGDRVTMTCKASQSVSNDVAWYQQ | 135 |
| | | KPGQSPKLLIYYVSNRYIGVPDRFTGSGYGTDFTFTIS | |
| | | TVQAEDLAVYFCQQDYSSPPTFGGGTKLEIK | |
| 18 | 5T4_VH_ | EVQLQQSGPDLVKPGASVRISCKASGYSFTGYYIHWVK | 136 |
| | | QSHGKSLEWIGRVNPNNGGTIYNQKFKGKAILTVDKSS | |
| | | NTASMEYRSLTSEDSAVYYCARSTMITSYYFDYWGQGT | |
| | | TLTVSS | |
| | 5T4_VL_ | NIVMTQTPKFLLVSAGDRVTITCKASQSVNYDVAWYQQ | 137 |
| | | KPGQSPKPLIYYASKRYTGVPDRFTGSGFGTDFTFTIN | |
| | | TVQAGDLAIYFCQQDYSSPPTFGGGTKLEIK | |
| 19 | 5T4_VH_ | QVOLQQSGAELMKPGASVRISCKATGYTFSTYWIEWVR | 138 |
| | | QRPGHGLEWIGEILPGSGRNNYNEKFKGKATFTADTSS | |
| | | NTAYIQLSSLTSEDSAVYYCAKGNYGSSPYYFDYWGQG | |
| | | TTLTVSS | |
| | 5T4_VL_ | DIVLTQSPASLVVSLGQRATISCRASQSVSTSRNSYMH | 139 |
| | | WYQQKPGQPPKVLIKYASNLESGVPARFSGSGSGTDFT | |
| | | LNIHPVEEEDTATYYCQHSWEIPLTFGAGTKLELK | |
| 20 | 5T4_VH_ | QVTLKESGPGILQPSQTLSLTCSFSGFSLSTSGMGVGW | 140 |
| | | IRQPSGKGLEWLAHIWWDDVKRYNPALKSRLTISKDTS | |
| | | SSQVFLNIASVDTADTATYYCARIADGYYAPWFAYWGQ | |
| | | GTLVTVSA | |
| | 5T4_VL_ | DIQMTQSPSSLSASLGGKVTITCKASQDINKYIAWYQH | 141 |
| | | KPGKGPRLIIHYTSTLQPDIPSRFSGSGSGRDYSFSIS | |
| | | NLEPEDIATYFCLQCDDLWTFGGGTKIEIK | |
| 3 | 5T4-VH | EVKLLESGGGLVQPGGSLKLSCAASGFDFSRYWMTWVR | 156 |
| | | QAPGKGLEWIGEINPDSNTINYTPSLKDKFIISRDNAK | |
| | | NTLYLQLTKVRSEDTGLYYCASPDYDYNPYYFAYWGQG | |
| | | TPLTVSS | |
| | 5T4_VL | DIVVTQSPASLAVSLGQRATISCRASQSVSTSRYSYMH | 157 |
| | | WYQQKPGQSPKLLIKYASNLESGVPPRFSGSGSGTDFT | |
| | | LNIHPVEEEDTATYYCQHSWEIPLTFGAGTKLELK | |
| 4 | 5T4-VH | DVQLVESGGGLVQPGGSRTLSCAASGFTFSSFGLHWVR | 158 |
| | | QSPEKGLEWVAYISVGSSTIYYADPVKGRFTISRDNPK | |
| | | NTLFLQMTSLRSEDTAIYYCARSRTYYRSEIDSWGQGT | |
| | | TLTVSS | |
| | 5T4_VL | DIVLTQSPASLTVSLGQRATISCRASQSVSSSSYNYMH | 159 |
| | | WYROKPGQPPKLLIKSASTLESGVPARFSGSGSGTDFT | |
| | | LNIHPVEEEDTATYYCQHSWEIPYTFGGGTKLEIK | |
| 5 | 5T4-VH | EVQLQQSGAELVKPGASVKLSCTASGFNIKDTYMHWVK | 160 |
| | | QRPEQGLEWIGRIDPADGNTKFDPKFQGKATITADTSS | |
| | | NTAYLQLSSLTSEDTAVYYCARFRRYDYVMDYWGQGTS | |
| | | VTVSS | |
| | 5T4_VL | NIVLTQSPASLAVSLGQRATISCRASESVDSYGNIFMH | 161 |
| | | WYQQIPGQPPKLLIYLASNLESGVPARFSGSGSRTDFT | |
| | | LTIDPVEADDAATYYCQQNNEDPWTFGGGTKLEIK | |
| 1 | 5T4-VH | EVQLQQSGPDLVKPGASVKISCKASGYSFTDYYMHWVK | 162 |
| | | QSHGKSLEWIGRVSPNNGATNTNQKFKDKAILTVDKSS | |
| | | STAYMDLRSLTSEDSAVYYCARSTMITSYYFDYWGQGT | |
| | | TLTVSS | |
| | 5T4_VL | SIVMTQTPKFLLVSAGDRITITCKASQSVSTDVAWYQQ | 163 |
| | | KPGQSPKLLIYFASDRYTGVPDRFTGSGNGTDFTFTIT | |
| | | TVQAEDLAIYFCQQDYSSPPTFGGGTKLEIK | |
| 6 | 5T4-VH | EVQLQQSGPDLVKPGASVRISCKASGYSFTGYYLHWVK | 164 |
| | | QSHGKSLEWIGRVNPNNGATVYNQNFKGKAVLTVDKSS | |
| | | STAYMELRSLTSEDSAVYYCVRSIMITTYDFDYWGQGT | |
| | | TLTVSS | |
| | 5T4_VL | DIVMTQSHKFMSTSVGDRVSITCKASQDVSIDVGWYQQ | 165 |
| | | KPGQSPKLLIYWASTRHTGVPDRFTGSGSGTDYTLTIS | |
| | | NVQAEDLALYYCQQHHIVPPTFGSGTKLEIK | |
| 7 | 5T4-VH | EVQLQQSGPDLVRPGASVKISCKASGYSFTGYYLHWVK | 166 |
| | | QSHGKSLEWIGRVNPNNGGTIYNQNFKGKAMLNVDKSS | |
| | | TTAYMELRSLTSEDSAVYYCARSIMITTFDFDYWGQGT | |
| | | TLTVSS | |
| | 5T4_VL | DIVMTQSHKFMSTSVGDRVSITCKASQDVNTAVVWYQQ | 167 |
| | | KPGQSPELLIYWASTRHTGVPDRFSGSGSGTDYTLTIS | |
| | | SVQAEDLALYYCQQHYSTPPTFGSGTKLEIK | |
| 8 | 5T4-VH | QVQLQQSGAELMKPGASVKISCKATGYTFSSYWIEWVR | 168 |
| | | QRPGHGLEWIGEILPGSGRTNYNEKFKGKATFTADTSS | |
| | | NTAYIQLSSLTSEDSAVYYCAKGNYGSSPYYFDYWGQG | |
| | | TTLTVSS | |
| | 5T4_VL | DIVLTQSPASLAVSLGQRATISCRASQSVSTSRNSYMH | 169 |
| | | WYQQKPGQPPKVLIKYASNLESGVPARFSGSGSGTDFT | |
| | | LNIHPVEEEDTATYYCQHSWEIPLTFGAGTKLELK | |
| 9 | 5T4-VH | QVQLQQSGPELVKPGASVKMSCKASGYTFTHYVISWVK | 170 |
| | | QRTGQGLEWIGEIYPGSGSTYYNEKFKGKATLTADKSS | |
| | | NTAYMQLSSLTSEDSAVYFCARGGRYGFDYWGQGTTLT | |
| | | VSS | |
| | 5T4_VL | QIVLTQSPAIMSASLGERVTMTCTASSSVSSSYLHWYQ | 171 |
| | | QKPGSSPKLWIYSTSNLASGVPARFSGSGSGTSYSLTI | |
| | | SSMEAEDAVTYYCHQYHRSPLTFGAGTKLELK | |
| 10 | 5T4-VH | QVTLKESGPGILQPSQTLSLTCSFSGFSLSTSGVGVDW | 172 |
| | | IRQPSGKGLEWLAHIWWDDVKRYNPALKSRLTISKDTS | |
| | | SSQVFLKIASVDTADTATYYCARIADGYSAPWFAYWGQ | |
| | | GTLVTVSA | |
| | 5T4_VL | DIQMTQSPSSLSASLGGKVTITCKASQDINKYIAWYQH | 173 |
| | | KPGKGPRLLIHYTSTLQPGIPSRFSGSGSGRDYSFSIS | |
| | | NLEPEDIATYYCLQCDNLWTFGGGTKLEIK | |

The disclosure provides antigen binding domains, antibodies, bispecific antibodies (e.g. T cell bispecific antibodies), antibody-drug conjugates, and chimeric receptors (CARs) comprising antigen binding domains comprising the variable heavy domains, paired with any of the variable light chain domains, set forth in Tables 8 and 9. In some embodiments, the antibody or antibody-drug conjugate comprises a biparatopic antibody comprising a first antigen binding domain that specifically binds to a first 5T4 epitope, a second antigen binding domain that specifically binds to a second 5T4 epitope that is not the same as the first 5T4 epitope, wherein the first antigen binding domain is operably linked to the second antigen binding domain, and a chemotherapeutic agent. In some embodiments, the first and/or second antigen binding domains comprise any of the variable heavy domains, paired with any of the variable light chain domains set forth in Tables 8 and 9.

Provided herein are antibody-drug conjugates that bind to at least one epitope of a 5T4 protein. In some embodiments, the antibody-drug conjugates bind to one epitope of a 5T4 antigen. In some embodiments, the antibody-drug conjugates bind to two epitopes of a 5T4 antigen.

In some embodiments, the antibody-drug conjugates comprise a first antigen binding domain that specifically binds to a first 5T4 epitope; a second antigen binding domain that specifically binds to a second 5T4 epitope that is not the same as the first 5T4 epitope; wherein the first antigen binding domain is operably linked to the second antigen binding domain In some embodiments, the first and second antigen binding domains are independently selected from the group consisting of a Fab fragment, a F(ab′)2 fragment, a scFv, a scab, a dAb, a single domain heavy chain antibody, a single domain light chain antibody, and a full-length IgG antibody. In some embodiments, the first antigen binding domain comprises a full-length IgG antibody. In some embodiments, the full-length IgG antibody comprises two heavy chains and two light chains. In some embodiments of the antibody-drug conjugates disclosed herein, the second antigen binding domain comprises an scFv. In some embodiments, the antibody-drug conjugates comprise two second antigen binding domain scFv that both specifically bind the second 5T4 epitope.

In some embodiments, the scFv comprises a heavy chain and a light chain. In some embodiments, the C-terminus of the light chain is operably linked to the N-terminus of the heavy chain via a linker, or the C-terminus of the heavy chain is operably linked to the N-terminus of the light chain via a linker. In some embodiments, the linker connecting the heavy chain and light chain comprises a sequence of SEQ ID NO: 153.

In some embodiments, the N-terminus of the second antigen binding domain is operably linked to the C-terminus of a heavy chain of the first antigen binding domain. In some embodiments, the C-terminus of the second antigen binding domain is operably linked to the N-terminus of a heavy chain of the first antigen binding domain. In some embodiments, the second antigen binding domain is operably linked to the heavy chain of the first antigen binding domain using a linker. In some embodiments, the linker comprises or consists of an amino acid sequence of SEQ ID NO: 152.

### Antibody Constant Region Domain

Antibodies, including monoclonal, bispecific and biparatopic antibodies comprising full length IgG antibodies, are contemplated as within the scope of the instant disclosure. In some embodiments, a full length IgG antibody comprises two heavy chains and two light chains, each comprising a variable region domain and a constant region domain, as described in more detail below. The skilled artisan will appreciate that monoclonal antibodies, as well as bispecific and biparatopic antibodies comprising full length IgG antibodies having the arrangement of variable and constant domains described below are contemplated as within the scope of the instant disclosure.

In some embodiments, the constant region domain is an IgG1 isotype constant region domain. In some embodiments, the constant region domain comprises an amino acid sequence of SEQ ID NO: 148.

In some embodiments, the antibody light chains comprise a variable region domain and a constant region domain. In some embodiments, the constant region domain is an IgG1 isotype constant region domain. In some embodiments, the light chain comprises an IgG1 isotype constant region domain, the constant region domain comprises an amino acid sequence of SEQ ID NO: 149.

In some embodiments, for example those embodiments wherein the antibody comprises an IgG1 constant region domain, the IgG1 constant region domain comprises at least one mutation that reduces effector function, extends half-life, or a combination thereof.

In some embodiments, the at least one mutation comprises an F at position 237 relative to SEQ ID NO: 100 (L234F), a C or A at position 242 relative to SEQ ID NO: 100 (S239C/A), an A at position 437 relative to SEQ ID NO: 100 (N434A), or a combination thereof. In some embodiments, the constant region domain comprises an F at position 237 relative to SEQ ID NO: 100 (L234F), a C or A at position 242 relative to SEQ ID NO: 100 (S239C/A), and an A at position 437 relative to SEQ ID NO: 100 (N434A). In some embodiments, the at least one mutation comprises an F at position 237 relative to SEQ ID NO: 100 (L234F). In some embodiments, the at least one mutation comprises a C or A at position 242 relative to SEQ ID NO: 100 (S239C/A). In some embodiments, the at least one mutation comprises a C at position 242 relative to SEQ ID NO: 100 (S239C/A). In some embodiments, the at least one mutation comprises an A at position 242 relative to SEQ ID NO: 100 (S239C/A). In some embodiments, the at least one mutation comprises an A at position 437 relative to SEQ ID NO: 100 (N434A).

In some embodiments, the antibody heavy chain(s) comprise a constant region domain comprising an amino acid sequence of SEQ ID NO: 148, or a sequence having at least 80%, at least 85%, at least 90%, at least 95%, at least 97% or at least 99% identity thereto. In some embodiments, the antibody light chain(s) comprise a constant region domain comprising an amino acid sequence of SEQ ID NO: 149, or a sequence having at least 80%, at least 85%, at least 90%, at least 95%, at least 97% or at least 99% identity thereto. In some embodiments, the antibody heavy chain(s) comprise a constant region domain comprising an amino acid sequence of SEQ ID NO: 148, or a sequence having at least 80%, at least 85%, at least 90%, at least 95%, at least 97% or at least 99% identity thereto; and the first antibody light chain(s) comprise a constant region domain comprising an amino acid sequence of SEQ ID NO: 149, or a sequence having at least 80%, at least 85%, at least 90%, at least 95%, at least 97% or at least 99% identity thereto.

In some embodiments, the antibody heavy chain(s) comprise a constant region domain comprising an amino acid sequence of SEQ ID NO: 148. In some embodiments, the antibody light chain(s) comprise a constant region domain comprising an amino acid sequence of SEQ ID NO: 149. In some embodiments, the antibody heavy chain(s) comprise a constant region domain comprising an amino acid sequence of SEQ ID NO: 148; and the first antibody light chain(s) comprise a constant region domain comprising an amino acid sequence of SEQ ID NO: 149.

In some embodiments of the antibody-drug conjugate disclosed herein, the antibody-drug conjugate comprises a full-length IgG antibody comprising a first antigen binding domain and a second antigen binding domain comprising an scFv. In some embodiments, the antibody-drug conjugate comprises four polypeptides. In some embodiments, the antibody-drug conjugate comprises two polypeptides comprising, from N to C terminus, the IgG antibody heavy chain, a linker, and the second antigen binding domain. In some embodiments, the antibody-drug conjugate comprises four polypeptides comprising (a) two polypeptides comprising, from N to C terminus, the full length IgG antibody heavy chain, a linker, and the second antigen binding domain; and (b) two polypeptides comprising the full length IgG antibody light chain. In some embodiments, the linker connecting the full length IgG antibody heavy chain and the second antigen binding domain comprises an amino acid sequence of SEQ ID NO: 152.

In some embodiments of the antibody-drug conjugate disclosed herein, the antibody-drug conjugate comprises a full-length IgG antibody comprising a first antigen binding domain and a second antigen binding domain comprising an scFv, and the antibody-drug conjugate comprises two polypeptides comprising, from N to C terminus, the second antigen binding domain, a linker, and the full length IgG antibody heavy chain. In some embodiments, the first antigen binding domain comprises a full-length IgG antibody and the second antigen binding domain comprises an scFv, and the antibody-drug conjugate comprises four polypeptides comprising: (a) two polypeptides comprising, from N to C terminus, the second antigen binding domain, a linker, and the first antigen binding domain heavy chain; and (b) two polypeptides comprising the first antigen binding domain light chain. In some embodiments, the linker connecting the second antigen binding domain and the first antigen binding domain heavy chain comprises an amino acid sequence of SEQ ID NO: 152.

### Heavy Chain CDRs

In some embodiments, the antigen binding domains, as well as antibodies, antibody-drug conjugates and receptors comprising same described herein comprise a heavy chain (HC) complementarity determining region (CDR1) sequence selected from the group consisting of SEQ ID NOs: 1, 4, and 13-23 or a sequence having 1, 2 or 3 substitutions, insertions or deletions relative thereto; a HC CDR2 sequence selected from the group consisting of SEQ ID NOs: 2, 5, and 24-26 and 28-39 or a sequence having 1, 2 or 3 substitutions, insertions or deletions relative thereto; and a HC CDR3 sequence selected from the group consisting of SEQ ID NOs: 3, 6, and 40-52 or a sequence having 1, 2 or 3 substitutions, insertions or deletions relative thereto.

In some embodiments, the antigen binding domains described herein comprise a heavy chain (HC) complementarity determining region (CDR1) sequence selected from the group consisting of SEQ ID NOs: 1, 4, and 13-23; a HC CDR2 sequence selected from the group consisting of SEQ ID NOs: 2, 5, 24-26 and 28-39; and a HC CDR3 sequence comprising an amino acid sequence of SEQ ID NO: 3.

In some embodiments, the antigen binding domains described herein comprise a heavy chain (HC) complementarity determining region (CDR1) sequence selected from the group consisting of SEQ ID NOs: 1, 4, and 13-23; a HC CDR2 sequence selected from the group consisting of SEQ ID NOs: 2, 5, 24-26 and 28-39; and a HC CDR3 sequence comprising an amino acid sequence of SEQ ID NOs: 3, 6, and 40-52.

In some embodiments, the antibodies and antibody-drug conjugates described herein comprise a first and an at least second antigen binding domain, wherein the first and/or second antigen binding domain comprises a heavy chain comprising a heavy chain (HC) complementarity determining region (CDR1) sequence selected from the group consisting of SEQ ID NOs: 1, 4, and 13-23 or a sequence having 1, 2 or 3 substitutions, insertions or deletions relative thereto; a HC CDR2 sequence selected from the group consisting of SEQ ID NOs: 2, 5, 24-26 and 28-39 or a sequence having 1, 2 or 3 substitutions, insertions or deletions relative thereto; and a HC CDR3 sequence selected from the group consisting of SEQ ID NOs: 3, 6, and 40-52 or a sequence having 1, 2 or 3 substitutions, insertions or deletions relative thereto, wherein one or more of the CDR1, CDR2 and CDR3 sequences are not the same between the first and second antigen binding domains.

In some embodiments, the antibodies and antibody-drug conjugates described herein comprise a first and an at least second antigen binding domain, wherein the first and/or second antigen binding domain comprises a heavy chain comprising a heavy chain (HC) complementarity determining region (CDR1) sequence selected from the group consisting of SEQ ID NOs: 1, 4, and 13-23; a HC CDR2 sequence selected from the group consisting of SEQ ID NOs: 2, 5, 24-26 and 28-39; and a HC CDR3 sequence selected from the group consisting of SEQ ID NOs: 3, 6, and 40-52, wherein one or more of the CDR1, CDR2 and CDR3 sequences are not the same between the first and second antigen binding domains.

In some embodiments, the antibodies and antibody-drug conjugates described herein comprise an antigen biding domain comprising a heavy chain (HC) complementarity determining region (CDR1) sequence selected from any heavy chain CDR1 (CDRH1) sequence listed in Table 1; a HC CDR2 selected from any heavy chain CDR2 (CDRH2) sequence listed in Table 1; and a HC CDR3 selected from any heavy chain CDR3 (CDRH3) sequence listed in Table 1.

Those of ordinary skill in the art would understand that the antigen binding domains, antibodies, antibody-drug conjugates and receptors of the present disclosure can comprise a heavy chain comprising any CDRH1, CDRH2, or CDRH3 sequence listed in Table 1. In some embodiments, the heavy chain can comprise a combination of one CDRH1, one CDRH2, and one CDRH3 within a single row of Table 1. In some embodiments, the antibody-drug conjugates can comprise a heavy chain comprising a combination of one CDRH1, one CDRH2, and one CDRH3 wherein the CDRH1, CDRH2, and CDRH3 are not in the same row in Table 1. Those of ordinary skill in the art would understand that the antibody-drug conjugate of the present disclosure can comprise a heavy chain comprising any one CDRH1 sequence listed in Table 1, in combination with any one CDRH2 sequence listed in Table 1, in combination with any one CDRH3 sequence listed in Table 1.

In some embodiments, the heavy chain variable region domain comprises: (a) a HC CDR1 comprising SEQ ID NO: 1, a HC CDR2 comprising SEQ ID NO: 2, and a HC CDR3 comprising SEQ ID NO: 3; (b) a HC CDR1 comprising SEQ ID NO: 4, a HC CDR2 comprising SEQ ID NO: 5, and a HC CDR3 comprising SEQ ID NO: 6; (c) a HC CDR1 comprising SEQ ID NO: 4, a HC CDR2 comprising SEQ ID NO: 24, and a HC CDR3 comprising SEQ ID NO: 40; (d) a HC CDR1 comprising SEQ ID NO: 13, a HC CDR2 comprising SEQ ID NO: 25, and a HC CDR3 comprising SEQ ID NO: 41; (e) a HC CDR1 comprising SEQ ID NO: 14, a HC CDR2 comprising SEQ ID NO: 26, and a HC CDR3 comprising SEQ ID NO: 42; (f) a HC CDR1 comprising SEQ ID NO: 15, a HC CDR2 comprising SEQ ID NO: 28, and a HC CDR3 comprising SEQ ID NO: 43; (g) a HC CDR1 comprising SEQ ID NO: 15, a HC CDR2 comprising SEQ ID NO: 29, and a HC CDR3 comprising SEQ ID NO: 44; (h) a HC CDR1 comprising SEQ ID NO: 16, a HC CDR2 comprising SEQ ID NO: 30, and a HC CDR3 comprising SEQ ID NO: 45; (i) a HC CDR1 comprising SEQ ID NO: 17, a HC CDR2 comprising SEQ ID NO: 31, and a HC CDR3 comprising SEQ ID NO: 46; (j) a HC CDR1 comprising SEQ ID NO: 18, a HC CDR2 comprising SEQ ID NO: 32, and a HC CDR3 comprising SEQ ID NO: 47; (k) a HC CDR1 comprising SEQ ID NO: 19, a HC CDR2 comprising SEQ ID NO: 33, and a HC CDR3 comprising SEQ ID NO: 48; (1) a HC CDR1 comprising SEQ ID NO: 20, a HC CDR2 comprising SEQ ID NO: 34, and a HC CDR3 comprising SEQ ID NO: 49; (m) a HC CDR1 comprising SEQ ID NO: 4, a HC CDR2 comprising SEQ ID NO: 35, and a HC CDR3 comprising SEQ ID NO: 50; (n) a HC CDR1 comprising SEQ ID NO: 14, a HC CDR2 comprising SEQ ID NO: 36, and a HC CDR3 comprising SEQ ID NO: 51; (o) a HC CDR1 comprising SEQ ID NO: 15, a HC CDR2 comprising SEQ ID NO: 37, and a HC CDR3 comprising SEQ ID NO: 3; (p) a HC CDR1 comprising SEQ ID NO: 21, a HC CDR2 comprising SEQ ID NO: 38, and a HC CDR3 comprising SEQ ID NO: 3; (q) a HC CDR1 comprising SEQ ID NO: 22, a HC CDR2 comprising SEQ ID NO: 39, and a HC CDR3 comprising SEQ ID NO: 45; or (r) a HC CDR1 comprising SEQ ID NO: 23, a HC CDR2 comprising SEQ ID NO: 32, and a HC CDR3 comprising SEQ ID NO: 52.

### Light Chain CDRs

In some embodiments, the antigen binding domains, as well as antibodies, antibody-drug conjugates and receptors comprising same described herein comprise a light chain (LC) complementarity determining region (CDR1) sequence selected from the group consisting of SEQ ID NOs: 7, 10, and 53-66 or a sequence having 1, 2 or 3 substitutions, insertions or deletions relative thereto; a LC CDR2 sequence selected from the group consisting of SEQ ID NOs: 8, 11, and 67-75 or a sequence having 1, 2 or 3 substitutions, insertions or deletions relative thereto; a LC CDR3 sequence selected from the group consisting of SEQ ID NOs: 9, 12, and 76-83 or a sequence having 1, 2 or 3 substitutions, insertions or deletions relative thereto.

In some embodiments, the antibody-drug conjugates described herein comprise a first and a second antigen binding domain, wherein the first and/or second antigen binding domain comprises a light chain (LC) complementarity determining region (CDR1) sequence selected from the group consisting of SEQ ID NOs: 7, 10, and 53-66 or a sequence having 1, 2 or 3 substitutions, insertions or deletions relative thereto; a LC CDR2 sequence selected from the group consisting of SEQ ID NOs: 8, 11, and 67-75 or a sequence having 1, 2 or 3 substitutions, insertions or deletions relative thereto; a LC CDR3 sequence selected from the group consisting of SEQ ID NOs: 9, 12, and 76-83 or a sequence having 1, 2 or 3 substitutions, insertions or deletions relative thereto, wherein one or more of the CDR1, CDR2 and CDR3 sequences are not the same between the first and second antigen binding domains.

In some embodiments, the antibody-drug conjugates described herein comprise a first and a second antigen binding domain, wherein the first and/or second antigen binding domain comprises a light chain (LC) complementarity determining region (CDR1) sequence selected from the group consisting of SEQ ID NOs: 7, 10, and 53-66; a LC CDR2 sequence selected from the group consisting of SEQ ID NOs: 8, 11, and 67-75; a LC CDR3 sequence selected from the group consisting of SEQ ID NOs: 9, 12, and 76-83, wherein one or more of the CDR1, CDR2 and CDR3 sequences are not the same between the first and second antigen binding domains.

In some embodiments, the first antigen binding domain comprises a heavy chain variable region domain comprising a HC CDR1 sequence comprising an amino acid sequence of SEQ ID NO: 1, or a sequence having 1, 2 or 3 substitutions, insertions or deletions relative thereto; a HC CDR2 sequence comprising an amino acid sequence of SEQ ID NO: 2, or a sequence having 1, 2 or 3 substitutions, insertions or deletions relative thereto; and a HC CDR3 sequence comprising an amino acid sequence of SEQ ID NO: 3 or a sequence having 1, 2 or 3 substitutions, insertions or deletions relative thereto.

In some embodiments, the first antigen binding domain comprises a light chain variable region domain comprising a LC CDR1 sequence comprising an amino acid sequence of SEQ ID NO: 7, or a sequence having 1, 2 or 3 substitutions, insertions or deletions relative thereto; a LC CDR2 sequence comprising an amino acid sequence of SEQ ID NO: 8, or a sequence having 1, 2 or 3 substitutions, insertions or deletions relative thereto; and a LC CDR3 sequence comprising an amino acid sequence of SEQ ID NO: 9, or a sequence having 1, 2 or 3 substitutions, insertions or deletions relative thereto.

In some embodiments, the first antigen binding domain comprises a heavy chain variable region domain comprising a HC CDR1 sequence comprising an amino acid sequence of SEQ ID NO: 1; a HC CDR2 sequence comprising an amino acid sequence of SEQ ID NO: 2; and a HC CDR3 sequence comprising an amino acid sequence of SEQ ID NO: 3. In some embodiments, the first antigen binding domain comprises a light chain variable region domain comprising a LC CDR1 sequence comprising an amino acid sequence of SEQ ID NO: 7; a LC CDR2 sequence comprising an amino acid sequence of SEQ ID NO: 8; and a LC CDR3 sequence comprising an amino acid sequence of SEQ ID NO: 9.

In some embodiments, the first antigen binding domain comprises a heavy chain variable region domain comprising a HC CDR1 sequence comprising an amino acid sequence of SEQ ID NO: 1; a HC CDR2 sequence comprising an amino acid sequence of SEQ ID NO: 2; a HC CDR3 sequence comprising an amino acid sequence of SEQ ID NO: 3; and a light chain variable region domain comprising a LC CDR1 sequence comprising an amino acid sequence of SEQ ID NO: 7; a LC CDR2 sequence comprising an amino acid sequence of SEQ ID NO: 8; and a LC CDR3 sequence comprising an amino acid sequence of SEQ ID NO: 9.

In some embodiments of the antibody-drug conjugate, the first antigen binding domain comprises a heavy chain variable region domain comprising a HC CDR1 sequence comprising an amino acid sequence of SEQ ID NO: 1; a HC CDR2 sequence comprising an amino acid sequence of SEQ ID NO: 2; a HC CDR3 sequence comprising an amino acid sequence of SEQ ID NO: 3; and a light chain variable region domain comprising a LC CDR1 sequence comprising an amino acid sequence of SEQ ID NO: 10; a LC CDR2 sequence comprising an amino acid sequence of SEQ ID NO: 11; and a LC CDR3 sequence comprising an amino acid sequence of SEQ ID NO: 12.

In some embodiments, the second antigen binding domain comprises a heavy chain variable region domain comprising a HC CDR1 sequence comprising an amino acid sequence of SEQ ID NO: 4; a HC CDR2 sequence comprising an amino acid sequence of SEQ ID NO: 5; a HC CDR3 sequence comprising an amino acid sequence of SEQ ID NO: 6. In some embodiments, the second antigen binding domain comprises a light chain variable region domain comprising a LC CDR1 sequence comprising an amino acid sequence of SEQ ID NO: 10; a LC CDR2 sequence comprising an amino acid sequence of SEQ ID NO: 11; and a LC CDR3 sequence comprising an amino acid sequence of SEQ ID NO: 12.

In some embodiments, the first antigen binding domain comprises a heavy chain variable region domain comprising a HC CDR1 sequence comprising an amino acid sequence of SEQ ID NO: 1; a HC CDR2 sequence comprising an amino acid sequence of SEQ ID NO: 2; and a HC CDR3 sequence comprising an amino acid sequence of SEQ ID NO: 3; and a light chain variable region domain comprising a LC CDR1 sequence comprising an amino acid sequence of SEQ ID NO: 7; a LC CDR2 sequence comprising an amino acid sequence of SEQ ID NO: 8; and a LC CDR3 sequence comprising an amino acid sequence of SEQ ID NO: 9; and the second antigen binding domain comprises a heavy chain variable region domain comprising a HC CDR1 sequence comprising an amino acid sequence of SEQ ID NO: 4; a HC CDR2 sequence comprising an amino acid sequence of SEQ ID NO: 5; a HC CDR3 sequence comprising an amino acid sequence of SEQ ID NO: 6; and the light chain variable region domain comprising a LC CDR1 sequence comprising an amino acid sequence of SEQ ID NO: 10; a LC CDR2 sequence comprising an amino acid sequence of SEQ ID NO: 11; and a LC CDR3 sequence comprising an amino acid sequence of SEQ ID NO: 12.

In some embodiments, the first antigen binding domain comprises a heavy chain variable region domain comprising a HC CDR1 sequence comprising an amino acid sequence of SEQ ID NO: 1; a HC CDR2 sequence comprising an amino acid sequence of SEQ ID NO: 2; and a HC CDR3 sequence comprising an amino acid sequence of SEQ ID NO: 3; and a light chain variable region domain comprising a LC CDR1 sequence comprising an amino acid sequence of SEQ ID NO: 10; a LC CDR2 sequence comprising an amino acid sequence of SEQ ID NO: 11; and a LC CDR3 sequence comprising an amino acid sequence of SEQ ID NO: 12; and the second antigen binding domain comprises a heavy chain variable region domain comprising a HC CDR1 sequence comprising an amino acid sequence of SEQ ID NO: 4; a HC CDR2 sequence comprising an amino acid sequence of SEQ ID NO: 5; a HC CDR3 sequence comprising an amino acid sequence of SEQ ID NO: 6; and a light chain variable region domain comprising a LC CDR1 sequence comprising an amino acid sequence of SEQ ID NO: 7; a LC CDR2 sequence comprising an amino acid sequence of SEQ ID NO: 8; and a LC CDR3 sequence comprising an amino acid sequence of SEQ ID NO: 9.

Those of ordinary skill in the art will understand that the antigen binding domains, antibodies, antibody-drug conjugates, and receptors comprising same of the present disclosure can comprise a light chain comprising any CDRL1, CDRL2, or CDRL3 sequence listed in Table 2. In some embodiments, the light chain can comprise a combination of one CDRL1, one CDRL2, and one CDRL3 within a single row of Table 2. In some embodiments, the antigen binding domains comprise a light chain comprising a combination of one CDRL1, one CDRL2, and one CDRL3 wherein the CDRL1, CDRL2, and CDRL3 are not in the same row in Table 2. Those of ordinary skill in the art will understand that the antigen binding domains of the present disclosure can comprise a light chain comprising any one CDRL1 sequence listed in Table 2, in combination with any one CDRL2 sequence listed in Table 2, in combination with any one CDRL3 sequence listed in Table 2.

In some embodiments, the antigen binding domain comprises light chain variable region domain comprising: (a) a LC CDR1 comprising SEQ ID NO: 7, a LC CDR2 comprising SEQ ID NO: 8, and a LC CDR3 comprising SEQ ID NO: 9; (b) a LC CDR1 comprising SEQ ID NO: 10, a LC CDR2 comprising SEQ ID NO: 11, and a LC CDR3 comprising SEQ ID NO: 12; (c) a LC CDR1 comprising SEQ ID NO: 53, a LC CDR2 comprising SEQ ID NO: 11, and a LC CDR3 comprising SEQ ID NO: 76; (d) a LC CDR1 comprising SEQ ID NO: 54, a LC CDR2 comprising SEQ ID NO: 67, and a LC CDR3 comprising SEQ ID NO: 77; (e) a LC CDR1 comprising SEQ ID NO: 55, a LC CDR2 comprising SEQ ID NO: 68, and a LC CDR3 comprising SEQ ID NO: 78; (f) a LC CDR1 comprising SEQ ID NO: 56, a LC CDR2 comprising SEQ ID NO: 69, and a LC CDR3 comprising SEQ ID NO: 79; (g) a LC CDR1 comprising SEQ ID NO: 57, a LC CDR2 comprising SEQ ID NO: 69, and a LC CDR3 comprising SEQ ID NO: 79; (h) a LC CDR1 comprising SEQ ID NO: 58, a LC CDR2 comprising SEQ ID NO: 11, and a LC CDR3 comprising SEQ ID NO: 76; (i) a LC CDR1 comprising SEQ ID NO: 59, a LC CDR2 comprising SEQ ID NO: 70, and a LC CDR3 comprising SEQ ID NO: 80; (j) a LC CDR1 comprising SEQ ID NO: 60, a LC CDR2 comprising SEQ ID NO: 71, and a LC CDR3 comprising SEQ ID NO: 81; (k) a LC CDR1 comprising SEQ ID NO: 61, a LC CDR2 comprising SEQ ID NO: 72, and a LC CDR3 comprising SEQ ID NO: 77; (1) a LC CDR1 comprising SEQ ID NO: 62, a LC CDR2 comprising SEQ ID NO: 70, and a LC CDR3 comprising SEQ ID NO: 82; (m) a LC CDR1 comprising SEQ ID NO: 10, a LC CDR2 comprising SEQ ID NO: 73, and a LC CDR3 comprising SEQ ID NO: 12; (n) a LC CDR1 comprising SEQ ID NO: 63, a LC CDR2 comprising SEQ ID NO: 11, and a LC CDR3 comprising SEQ ID NO: 76; (o) a LC CDR1 comprising SEQ ID NO: 64, a LC CDR2 comprising SEQ ID NO: 68, and a LC CDR3 comprising SEQ ID NO: 78; (p) a LC CDR1 comprising SEQ ID NO: 65, a LC CDR2 comprising SEQ ID NO: 74, and a LC CDR3 comprising SEQ ID NO: 9; (q) a LC CDR1 comprising SEQ ID NO: 66, a LC CDR2 comprising SEQ ID NO: 75, and a LC CDR3 comprising SEQ ID NO: 9;® a LC CDR1 comprising SEQ ID NO: 58, a LC CDR2 comprising SEQ ID NO: 11, and a LC CDR3 comprising SEQ ID NO: 76; or (s) a LC CDR1 comprising SEQ ID NO: 60, a LC CDR2 comprising SEQ ID NO: 71, and a LC CDR3 comprising SEQ ID NO: 83.

In some embodiments, heavy chain variable region domain comprises a HC CDR1 comprising SEQ ID NO: 1, a HC CDR2 comprising SEQ ID NO: 2, and a HC CDR3 comprising SEQ ID NO: 3, and the light chain variable region domain comprises a LC CDR1 comprising SEQ ID NO: 7, a LC CDR2 comprising SEQ ID NO: 8, and a LC CDR3 comprising SEQ ID NO: 9.

In some embodiments, the heavy chain variable region domain comprises a HC CDR1 comprising SEQ ID NO: 4, a HC CDR2 comprising SEQ ID NO: 5, and a HC CDR3 comprising SEQ ID NO: 6, and the light chain variable region domain comprises a LC CDR1 comprising SEQ ID NO: 10, a LC CDR2 comprising SEQ ID NO: 11, and a LC CDR3 comprising SEQ ID NO: 12.

In some embodiments, the heavy chain variable region domain comprises a HC CDR1 comprising SEQ ID NO: 4, a HC CDR2 comprising SEQ ID NO: 24, and a HC CDR3 comprising SEQ ID NO: 40, and the light chain variable region domain comprises a LC CDR1 comprising SEQ ID NO: 53, a LC CDR2 comprising SEQ ID NO: 11, and a LC CDR3 comprising SEQ ID NO: 76.

In some embodiments, the heavy chain variable region domain comprises a HC CDR1 comprising SEQ ID NO: 13, a HC CDR2 comprising SEQ ID NO: 25, and a HC CDR3 comprising SEQ ID NO: 41, and the light chain variable region domain comprises a LC CDR1 comprising SEQ ID NO: 54, a LC CDR2 comprising SEQ ID NO: 67, and a LC CDR3 comprising SEQ ID NO: 77.

In some embodiments, the heavy chain variable region domain comprises a HC CDR1 comprising SEQ ID NO: 14, a HC CDR2 comprising SEQ ID NO: 26, and a HC CDR3 comprising SEQ ID NO: 42, and the light chain variable region domain comprises a LC CDR1 comprising SEQ ID NO: 55, a LC CDR2 comprising SEQ ID NO: 68, and a LC CDR3 comprising SEQ ID NO: 78.

In some embodiments, the heavy chain variable region domain comprises a HC CDR1 comprising SEQ ID NO: 15, a HC CDR2 comprising SEQ ID NO: 28, and a HC CDR3 comprising SEQ ID NO: 43, and the light chain variable region domain comprises a LC CDR1 comprising SEQ ID NO: 56, a LC CDR2 comprising SEQ ID NO: 69, and a LC CDR3 comprising SEQ ID NO: 79.

In some embodiments, the heavy chain variable region domain comprises a HC CDR1 comprising SEQ ID NO: 15, a HC CDR2 comprising SEQ ID NO: 29, and a HC CDR3 comprising SEQ ID NO: 44, and the light chain variable region domain comprises a LC CDR1 comprising SEQ ID NO: 57, a LC CDR2 comprising SEQ ID NO: 69, and a LC CDR3 comprising SEQ ID NO: 79.

In some embodiments, the heavy chain variable region domain comprises a HC CDR1 comprising SEQ ID NO: 16, a HC CDR2 comprising SEQ ID NO: 30, and a HC CDR3 comprising SEQ ID NO: 45, and the light chain variable region domain comprises a LC CDR1 comprising SEQ ID NO: 58, a LC CDR2 comprising SEQ ID NO: 11, and a LC CDR3 comprising SEQ ID NO: 76.

In some embodiments, the heavy chain variable region domain comprises a HC CDR1 comprising SEQ ID NO: 17, a HC CDR2 comprising SEQ ID NO: 31, and a HC CDR3 comprising SEQ ID NO: 46, and the light chain variable region domain comprises a LC CDR1 comprising SEQ ID NO: 59, a LC CDR2 comprising SEQ ID NO: 70, and a LC CDR3 comprising SEQ ID NO: 80.

In some embodiments, the heavy chain variable region domain comprises a HC CDR1 comprising SEQ ID NO: 18, a HC CDR2 comprising SEQ ID NO: 32, and a HC CDR3 comprising SEQ ID NO: 47, and the light chain variable region domain comprises a LC CDR1 comprising SEQ ID NO: 60, a LC CDR2 comprising SEQ ID NO: 71, and a LC CDR3 comprising SEQ ID NO: 81.

In some embodiments, the heavy chain variable region domain comprises a HC CDR1 comprising SEQ ID NO: 19, a HC CDR2 comprising SEQ ID NO: 33, and a HC CDR3 comprising SEQ ID NO: 48, and the light chain variable region domain comprises a LC CDR1 comprising SEQ ID NO: 61, a LC CDR2 comprising SEQ ID NO: 72, and a LC CDR3 comprising SEQ ID NO: 77.

In some embodiments, the heavy chain variable region domain comprises a HC CDR1 comprising SEQ ID NO: 20, a HC CDR2 comprising SEQ ID NO: 34, and a HC CDR3 comprising SEQ ID NO: 49, and the light chain variable region domain comprises a LC CDR1 comprising SEQ ID NO: 62, a LC CDR2 comprising SEQ ID NO: 70, and a LC CDR3 comprising SEQ ID NO: 82.

In some embodiments, the heavy chain variable region domain comprises a HC CDR1 comprising SEQ ID NO: 4, a HC CDR2 comprising SEQ ID NO: 35, and a HC CDR3 comprising SEQ ID NO: 50, and the light chain variable region domain comprises a LC CDR1 comprising SEQ ID NO: 10, a LC CDR2 comprising SEQ ID NO: 73, and a LC CDR3 comprising SEQ ID NO: 12.

In some embodiments, the heavy chain variable region domain comprises a HC CDR1 comprising SEQ ID NO: 4, a HC CDR2 comprising SEQ ID NO: 5, and a HC CDR3 comprising SEQ ID NO: 6, and the light chain variable region domain comprises a LC CDR1 comprising SEQ ID NO: 63, a LC CDR2 comprising SEQ ID NO: 11, and a LC CDR3 comprising SEQ ID NO: 76.

In some embodiments, the heavy chain variable region domain comprises a HC CDR1 comprising SEQ ID NO: 14, a HC CDR2 comprising SEQ ID NO: 36, and a HC CDR3 comprising SEQ ID NO: 51, and the light chain variable region domain comprises a LC CDR1 comprising SEQ ID NO: 64, a LC CDR2 comprising SEQ ID NO: 68, and a LC CDR3 comprising SEQ ID NO: 78.

In some embodiments, the heavy chain variable region domain comprises a HC CDR1 comprising SEQ ID NO: 15, a HC CDR2 comprising SEQ ID NO: 37, and a HC CDR3 comprising SEQ ID NO: 3, and the light chain variable region domain comprises a LC CDR1 comprising SEQ ID NO: 65, a LC CDR2 comprising SEQ ID NO: 74, and a LC CDR3 comprising SEQ ID NO: 9.

In some embodiments, the heavy chain variable region domain comprises a HC CDR1 comprising SEQ ID NO: 21, a HC CDR2 comprising SEQ ID NO: 38, and a HC CDR3 comprising SEQ ID NO: 3, and the light chain variable region domain comprises a LC CDR1 comprising SEQ ID NO: 66, a LC CDR2 comprising SEQ ID NO: 75, and a LC CDR3 comprising SEQ ID NO: 9.

In some embodiments, the heavy chain variable region domain comprises a HC CDR1 comprising SEQ ID NO: 22, a HC CDR2 comprising SEQ ID NO: 39, and a HC CDR3 comprising SEQ ID NO: 45, and the light chain variable region comprises a LC CDR1 comprising SEQ ID NO: 58, a LC CDR2 comprising SEQ ID NO: 11, and a LC CDR3 comprising SEQ ID NO: 76.

In some embodiments, the heavy chain variable region domain comprises a HC CDR1 comprising SEQ ID NO: 23, a HC CDR2 comprising SEQ ID NO: 32, and a HC CDR3 comprising SEQ ID NO: 52, and the light chain variable region domain comprises a LC CDR1 comprising SEQ ID NO: 60, a LC CDR2 comprising SEQ ID NO: 71, and a LC CDR3 comprising SEQ ID NO: 83.

### Heavy Chain Variable Region Domains

In some embodiments of the antigen binding domains, as well as antibodies, antibody-drug conjugates and receptors comprising same of the present disclosure, the antigen binding domain comprises at least one heavy chain variable region domain comprising a sequence selected from the group consisting of SEQ ID NOs: 96, 97, 102, 104, 106, 108, 112, 114, 116, 118, 120, 122, 124, 126, 128, 132, 134, 136, 138, 140, 156, 158, 160, 162, 164, 166, 169, 170, and 172, or a sequence having at least 80%, at least 85%, at least 90%, at least 95%, at least 97% or at least 99% identity thereto. In some embodiments, the antigen binding domain comprises a single heavy chain variable region domain comprising a sequence selected from the group consisting of SEQ ID NO: 96, 97, 102, 104, 106, 108, 112, 114, 116, 118, 120, 122, 124, 126, 128, 132, 134, 136, 138, 140, 156, 158, 160, 162, 164, 166, 169, 170, and 172, or a sequence having at least 80%, at least 85%, at least 90%, at least 95%, at least 97% or at least 99% identity thereto.

In some embodiments, the antibody or antibody-drug conjugate comprises at least two heavy chain variable region domains each comprising a sequence selected independently from the group consisting of SEQ ID NOs: 96, 97, 102, 104, 106, 108, 112, 114, 116, 118, 120, 122, 124, 126, 128, 132, 134, 136, 138, 140, 156, 158, 160, 162, 164, 166, 169, 170, and 172, or a sequence having at least 80%, at least 85%, at least 90%, at least 95%, at least 97% or at least 99% identity thereto. In some embodiments, the antibody or antibody-drug conjugate comprises two heavy chain variable region domains each comprising a sequence selected independently from the group consisting of SEQ ID NO: 96, 97, 102, 104, 106, 108, 112, 114, 116, 118, 120, 122, 124, 126, 128, 132, 134, 136, 138, 140, 156, 158, 160, 162, 164, 166, 169, 170, and 172, or a sequence having at least 80%, at least 85%, at least 90%, at least 95%, at least 97% or at least 99% identity thereto. In some embodiments, the two heavy chain variable region domains are not the same. In some embodiments, the antibody or antibody-drug conjugate comprises more than two heavy chain variable region domains comprising a sequence selected from the group consisting of SEQ ID NO: 96, 97, 102, 104, 106, 108, 112, 114, 116, 118, 120, 122, 124, 126, 128, 132, 134, 136, 138,140, 156, 158, 160, 162, 164, 166, 169, 170, and 172, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 96, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 97, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 102, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 104, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 106, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 108, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 112, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 114, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 116, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 118, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 120, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 122, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 124, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 126, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 128, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 132, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 134, or a sequence having at least 80%, at least 85%, at least 90%, at least 95%, at least 97% or at least 99% identity thereto.

In some embodiments, the antibody-drug conjugate comprises a heavy chain variable region domain comprising an amino acid sequence of SEQ ID NO: 136, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 138, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 140, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 156, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 158, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 160, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 162, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 164, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 166, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 168, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 170, or a sequence having at least 80%, at least 85%, at least 90%, at least 95%, at least 97% or at least 99% identity thereto.

In some embodiments, the antigen binding domain comprises a heavy chain variable region domain comprising an amino acid sequence of SEQ ID NO: 172, or a sequence having at least 80%, at least 85%, at least 90%, at least 95%, at least 97% or at least 99% identity thereto.

In some embodiments, for example those embodiments where the antigen binding domain is incorporated in a biparatopic antibody or antibody-drug conjugate, the first and/or second antigen binding domains comprise a heavy chain variable region domain comprising a sequence selected from the group consisting of SEQ ID NO: 96, 97, 102, 104, 106, 108, 112, 114, 116, 118, 120, 122, 124, 126, 128, 132, 134, 136, 138, 140, 56, 158, 160, 162, 164, 166, 169, 170, and 172, or a sequence having at least 80%, at least 85%, at least 90%, at least 95%, at least 97% or at least 99% identity thereto.

In some embodiments, the first antigen binding domain comprises a heavy chain variable region domain comprising an amino acid sequence of SEQ ID NO: 96, or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 97 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 102 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 104 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 106 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 108 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 112 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 114 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 116 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 118 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 120 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 122 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 124 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 126 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 128 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 132 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 134 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 136 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 138 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 140 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 156 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 158 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 160 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 162 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 164 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 166 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 168 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 170 or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 172 or a sequence having at least 80%, at least 85%, at least 90%, at least 95%, at least 97% or at least 99% identity thereto.

In some embodiments, the second antigen binding domain comprises a heavy chain variable region domain comprising an amino acid sequence of SEQ ID NO: 96 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 97 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 102 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 104 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 106 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 108 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 112 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 114 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 116 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 118 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 120 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 122 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 124 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 126 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 128 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 132 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 134 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 136 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 138 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 140 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 156 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 158 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 160 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 162 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 164 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 166 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 168 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 170 or a sequence having at least 80%, at least 85%, at least 90%, at least 95%, at least 97% or at least 99% identity thereto. In some embodiments, the second antigen binding domain comprises a heavy chain variable region domain comprising an amino acid sequence of SEQ ID NO: 172 or a sequence having at least 80%, at least 85%, at least 90%, at least 95%, at least 97% or at least 99% identity thereto.

### Light Chain Variable Region Domains

In some embodiments of the antigen binding domains, as well as the antibodies, antibody-drug conjugates and receptors comprising same of the present disclosure, the antibody-drug conjugate comprises a light chain variable region domain comprising a sequence selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto. In some embodiments, the antibody or antibody-drug conjugate comprises at least one light chain variable region domain comprising a sequence selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto.

In some embodiments, the antibody or antibody-drug conjugate comprises at least two light chain variable region domains each comprising a sequence independently selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto. In some embodiments, the antibody or antibody-drug conjugate comprises two light chain variable region domains each comprising a sequence selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto. In some embodiments, the two light chain variable region domains are not the same. In some embodiments of the antibodies or antibody-drug conjugates of the present disclosure, the antibody or antibody-drug conjugate comprises more than two light chain variable region domains comprising a sequence selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto.

In some embodiments, the antigen binding domain comprises a light chain variable region domain comprising a sequence selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto.

In some embodiments, the antigen binding domain comprises a light chain variable region domain comprising an amino acid sequence of SEQ ID NO: 98, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 99, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 103, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 105, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 107, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 109, or a sequence having at least 80%, at least 85%, at least 90%, at least 95%, at least 97% or at least 99% identity thereto. In some embodiments of the antigen binding domain of the present disclosure, the antigen binding domain comprises a light chain variable region domain comprising an amino acid sequence of SEQ ID NO: 113, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 115, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 117, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 119, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 121, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 123, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 125, or a sequence having at least 80%, at least 85%, at least 90%, at least 95%, at least 97% or at least 99% identity thereto. In some embodiments o, the antigen binding domain comprises a light chain variable region domain comprising an amino acid sequence of SEQ ID NO: 127, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 129, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 131, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 133, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 135, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 137, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 139, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 141, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 157, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 159, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 161, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 163, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 165, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 167, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 169, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 171, or a sequence having at least 80%, at least 85%, at least 90%, at least 95%, at least 97% or at least 99% identity thereto. In some embodiments, the antigen binding domain comprises a light chain variable region domain comprising an amino acid sequence of SEQ ID NO: 173, or a sequence having at least 80%, at least 85%, at least 90%, at least 95%, at least 97% or at least 99% identity thereto.

In some embodiments, the heavy chain variable region domain comprises an amino acid sequence of SEQ ID NO: 96, and the light chain variable region domain comprises an amino acid sequence of SEQ ID NO: 98.

In some embodiments, the heavy chain variable region domain comprises an amino acid sequence of SEQ ID NO: 97, and the light chain variable region domain comprises an amino acid sequence of SEQ ID NO: 99.

In some embodiments, the heavy chain variable region domain comprises an amino acid sequence of SEQ ID NO: 102, and the light chain variable region domain comprises an amino acid sequence of SEQ ID NO: 103.

In some embodiments, the heavy chain variable region domain comprises an amino acid sequence of SEQ ID NO: 104, and the light chain variable region domain comprises an amino acid sequence of SEQ ID NO: 105.

In some embodiments, the heavy chain variable region domain comprises an amino acid sequence of SEQ ID NO: 106, and the light chain variable region domain comprises an amino acid sequence of SEQ ID NO: 107.

In some embodiments, the heavy chain variable region domain comprises an amino acid sequence of SEQ ID NO: 108, and the light chain variable region domain comprises an amino acid sequence of SEQ ID NO: 109.

In some embodiments, the heavy chain variable region domain comprises an amino acid sequence of SEQ ID NO: 112, and the light chain variable region domain comprises an amino acid sequence of SEQ ID NO: 113.

In some embodiments, the heavy chain variable region domain comprises an amino acid sequence of SEQ ID NO: 114, and the light chain variable region domain comprises an amino acid sequence of SEQ ID NO: 115.

In some embodiments, the heavy chain variable region domain comprises an amino acid sequence of SEQ ID NO: 116, and the light chain variable region domain comprises an amino acid sequence of SEQ ID NO: 117.

In some embodiments, the heavy chain variable region domain comprises an amino acid sequence of SEQ ID NO: 118, and the light chain variable region domain comprises an amino acid sequence of SEQ ID NO: 119.

In some embodiments, the heavy chain variable region domain comprises an amino acid sequence of SEQ ID NO: 120, and the light chain variable region domain comprises an amino acid sequence of SEQ ID NO: 121.

In some embodiments, for example those embodiments where the antigen binding domain is incorporated in a biparatopic antibody or antibody-drug conjugate, the first and/or second antibodies comprise a light chain variable region domain comprising a sequence selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto.

In some embodiments, the first and second antigen binding domains comprise a light chain variable region domain comprise a sequence selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto.

In some embodiments, the first antigen binding domain comprises a light chain variable region domain comprising an amino acid sequence of SEQ ID NO: 98, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 99, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 103, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 105, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 107, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 109, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 113, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 115, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 117, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 119, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 121, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 123, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 125, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 127, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 129, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 131, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 133, or a sequence having at least 80%, at least 85%, at least 90%, at least 95%, at least 97% or at least 99% identity thereto. In some embodiments e, the first antigen binding domain comprises a light chain variable region domain comprising an amino acid sequence of SEQ ID NO: 135, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 137, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 139, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 141, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 157, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 159, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 161, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 163, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 165, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 167, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 169, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 171, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 173, or a sequence having at least 80%, at least 85%, at least 90%, at least 95%, at least 97% or at least 99% identity thereto.

In some embodiments, the second antigen binding domain comprises a light chain variable region domain comprising an amino acid sequence of SEQ ID NO: 98, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 99, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 103, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 105, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 107, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 109, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 113, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 115, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 117, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 119, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 121, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 123, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 125, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 127, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 129, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 131, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 133, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 135, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 137, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 139, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 141, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 157, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 159, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 161, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 163, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 165, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 167, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 169, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 171, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 173, or a sequence having at least 80%, at least 85%, at least 90%, at least 95%, at least 97% or at least 99% identity thereto.

In some embodiments of the antibodies antibody-drug conjugates of the present disclosure, the first and/or second antigen biding domains comprise a heavy chain variable region domain comprising a sequence selected from the group consisting of SEQ ID NO: 96, 97, 102, 104, 106, 108, 112, 114, 116, 118, 120, 122, 124, 126, 128, 132, 134, 136, 138, 140, 156, 158, 160, 162, 164, 166, 168, 170 and 172, or a sequence having at least 80%, at least 85%, at least 90%, at least 95%, at least 97% or at least 99% identity thereto; and a light chain variable region domain comprising a sequence selected from the group consisting of SEQ ID NO: 98, 99, 103, 105, 107, 109, 113, 115, 117, 119, 121, 123, 125, 127, 129, 131, 133, 135, 137, 139, 141, 157, 159, 161, 163, 165, 167, 169, 171 and 173, or a sequence having at least 80%, at least 85%, at least 90%, at least 95%, at least 97% or at least 99% identity thereto.

In some embodiments, the first antigen binding domain comprises a heavy chain variable region domain comprising an amino acid sequence of SEQ ID NO: 96 or 97. In some embodiments, the first antigen binding domain comprises alight chain variable region domain comprising an amino acid sequence of SEQ ID NO: 98 or 99. In some embodiments, the first antigen binding domain comprises a heavy chain variable region domain comprising an amino acid sequence of SEQ ID NO: 96 or 97, and a light chain variable region domain comprising an amino acid sequence of SEQ ID NO: 98 or 99.

In some embodiments, the first antigen binding domain comprises a heavy chain variable region domain comprising an amino acid sequence of SEQ ID NO: 96, or a sequence having at least 80%, at least 85%, at least 90%, at least 95%, at least 97% or at least 99% identity thereto. In some embodiments, the first antigen binding domain comprises a light chain variable region domain comprising an amino acid sequence of SEQ ID NO: 98, or a sequence having at least 80%, at least 85%, at least 90%, at least 95%, at least 97% or at least 99% identity thereto. In some embodiments, the first antigen binding domain comprises a heavy chain variable region domain comprising an amino acid sequence of SEQ ID NO: 96, or a sequence having at least 80%, at least 85%, at least 90%, at least 95%, at least 97% or at least 99% identity thereto; and a light chain variable region domain comprising an amino acid sequence of SEQ ID NO: 98, or a sequence having at least 80%, at least 85%, at least 90%, at least 95%, at least 97% or at least 99% identity thereto.

In some embodiments, the second antigen binding domain comprises a heavy chain variable region domain comprising an amino acid sequence of SEQ ID 97, or a sequence having at least 80%, at least 85%, at least 90%, at least 95%, at least 97% or at least 99% identity thereto. In some embodiments, the second antigen binding domain comprises a light chain variable region domain comprising an amino acid sequence of SEQ ID NO: 99, or a sequence having at least 80%, at least 85%, at least 90%, at least 95%, at least 97% or at least 99% identity thereto.

In some embodiments, the second antigen binding domain comprises a heavy chain variable region domain comprising an amino acid sequence of SEQ ID 97, or a sequence having at least 80%, at least 85%, at least 90%, at least 95%, at least 97% or at least 99% identity thereto; and a light chain variable region domain comprising an amino acid sequence of SEQ ID NO: 99, or a sequence having at least 80%, at least 85%, at least 90%, at least 95%, at least 97% or at least 99% identity thereto.

In some embodiments, the first antigen binding domain comprises a heavy chain variable region domain comprising an amino acid sequence of SEQ ID NO: 96, or a sequence having at least 80%, at least 85%, at least 90%, at least 95%, at least 97% or at least 99% identity thereto; and a light chain variable region domain comprising an amino acid sequence of SEQ ID NO: 98, or a sequence having at least 80%, at least 85%, at least 90%, at least 95%, at least 97% or at least 99% identity thereto; and the second antigen binding domain comprises a heavy chain variable region domain comprising an amino acid sequence of SEQ ID 97, or a sequence having at least 80%, at least 85%, at least 90%, at least 95%, at least 97% or at least 99% identity thereto; and a light chain variable region domain comprising an amino acid sequence of SEQ ID NO: 99, or a sequence having at least 80%, at least 85%, at least 90%, at least 95%, at least 97% or at least 99% identity thereto.

In some embodiments, the biparatopic antibody or antibody-drug conjugate comprises full length IgG1 antibody comprising the first antigen binding domain, and the antibody or antibody-drug conjugate comprises two polypeptides comprising a first antigen binding domain heavy chain variable region domain of SEQ ID NO: 96, a first linker of SEQ ID NO: 152, a second antigen binding domain heavy chain variable region domain of SEQ ID NO: 97, a second linker of SEQ ID NO: 153, and a light chain variable region of SEQ ID NO: 99. In some embodiments, the antibody or antibody-drug conjugate comprises two polypeptides comprising a light chain variable region domain of SEQ ID NO: 98. In some embodiments, the antibody-drug conjugate comprises two polypeptides comprising a first antigen binding domain heavy chain variable region domain of SEQ ID NO: 96, a first linker of SEQ ID NO: 152, a second antigen binding domain heavy chain variable region domain of SEQ ID NO: 97, a second linker of SEQ ID NO: 153, and a light chain variable region of SEQ ID NO: 99, and the antibody or antibody-drug conjugate further comprises two polypeptides comprising an antibody light chain variable region domain of SEQ ID NO: 98. In some embodiments, the heavy and light chains of the full length IgG1 comprise an IgG1 isotype constant region domain.

In some embodiments, the second antigen binding domain is an scFv comprising a first heavy chain domain comprising SEQ ID NO: 97, or a sequence having at least 80%, at least 85%, at least 90%, at least 95%, at least 97% or at least 99% identity thereto. In some embodiments, the second antigen binding domain is an scFv comprising a first heavy chain domain comprising SEQ ID NO: 97.

In some embodiments, the second antigen binding domain is an scFv comprising a light chain variable region domain comprising SEQ ID NO: 99, or a sequence having at least 80%, at least 85%, at least 90%, at least 95%, at least 97% or at least 99% identity thereto. In some embodiments, the second antigen binding domain is an scFv comprising a light chain variable region domain comprising SEQ ID NO: 99.

In some embodiments of the antibody-drug conjugate, the second antigen binding domain is an scFv comprising a first heavy chain domain comprising SEQ ID NO: 97; and a light chain variable region domain comprising SEQ ID NO: 99.

In some embodiments, the antibody or antibody-drug comprises a full-length IgG antibody comprising the first antigen binding domain, and the antigen binding domain antibody comprises an scFv, and the antibody or antibody-drug conjugate comprises two polypeptides comprising, from N to C terminus, a first antibody heavy chain variable region domain of SEQ ID NO: 96, an IgG1 isotype constant region domain of SEQ ID NO: 148, a first linker of SEQ ID NO: 152, a second antibody heavy chain variable region domain of SEQ ID NO: 97, a second linker of SEQ ID NO: 153, and a light chain variable region of SEQ ID NO: 99. In some embodiments, the antibody or antibody-drug conjugate comprises two polypeptides comprising, from N to C terminus, a first antibody heavy chain variable region domain of SEQ ID NO: 96, an IgG1 isotype constant region domain of SEQ ID NO: 148, a first linker of SEQ ID NO: 152, a second antibody light chain variable region of SEQ ID NO: 99, a second linker of SEQ ID NO: 153, and a heavy chain variable region domain of SEQ ID NO: 97.

In some embodiments, the antibody or antibody-drug conjugate comprises a full-length IgG antibody comprising the first antigen binding domain, and the second antigen binding domain comprises an scFv, and the antibody or antibody-drug conjugate comprises two polypeptides comprising, from N to C terminus, a first antibody variable light chain domain of SEQ ID NO: 98, and a first antibody light chain constant region domain of SEQ ID NO: 149.

In some embodiments, the antibody or antibody-drug conjugate comprises a full-length IgG antibody comprising the first antigen binding domain, and the second antigen binding domain comprises an scFv, and the antibody or antibody-drug conjugate comprises four polypeptides comprising: two polypeptides comprising, from N to C terminus, a first antigen binding domain heavy chain variable region domain of SEQ ID NO: 96, an IgG1 isotype constant region domain of SEQ ID NO: 148, a first linker of SEQ ID NO: 152, a second antigen binding domain heavy chain variable region domain of SEQ ID NO: 97, a second linker of SEQ ID NO: 153, and a light chain variable region of SEQ ID NO: 99; two polypeptides comprising, from N to C terminus, a first antigen binding domain variable light chain domain of SEQ ID NO: 98, and a first antigen binding domain light chain constant region domain of SEQ ID NO: 149.

In some embodiments, the antibody or antibody-drug conjugate comprises a full-length IgG antibody comprising the first antigen binding domain, and the second antigen binding domain comprises an scFv, and the antibody or antibody-drug conjugate comprises four polypeptides comprising: two polypeptides comprising, from N to C terminus, a first antigen binding domain heavy chain variable region domain of SEQ ID NO: 96, an IgG1 isotype constant region domain of SEQ ID NO: 148, a first linker of SEQ ID NO: 152, a second antigen binding domain light chain variable region of SEQ ID NO: 99, a second linker of SEQ ID NO: 153, and a heavy chain variable region domain of SEQ ID NO: 97; two polypeptides comprising, from N to C terminus, a first antigen binding domain variable light chain domain of SEQ ID NO: 98, and a first antigen binding domain light chain constant region domain of SEQ ID NO: 149.

In some embodiments, the antibody or antibody-drug conjugate comprises two polypeptides comprising a sequence of SEQ ID NO: 100, or a sequence having at least 80%, at least 85%, at least 90%, at least 95%, at least 97% or at least 99% identity thereto; and two polypeptides comprising a sequence of SEQ ID NO: 101, or a sequence having at least 80%, at least 85%, at least 90%, at least 95%, at least 97% or at least 99% identity thereto.

In some embodiments, the antibody or antibody-drug conjugate comprises two polypeptides comprising a sequence of SEQ ID NO: 100; and two polypeptides comprising a sequence of SEQ ID NO: 101.

The disclosure provides antibodies and antibody-drug conjugates, comprising a first antigen binding domain that specifically binds to a first 5T4 epitope; a second antigen binding domain that specifically binds to a second 5T4 epitope that is not the same as the first 5T4 epitope; wherein the first antigen binding domain is operably linked to the second antigen binding domain; and a chemotherapeutic agent, wherein the first antigen binding domain comprises a full-length IgG antibody and the second antigen binding domain comprises an scFv, and the antibody or antibody-drug conjugate comprises: (a) two polypeptides comprising at least one heavy chain variable region domain comprising: a HC CDR1 sequence comprising an amino acid sequence of SEQ ID NO: 1; a HC CDR2 sequence comprising an amino acid sequence of SEQ ID NO: 2; a HC CDR3 sequence comprising an amino acid sequence of SEQ ID NO: 3; and wherein the polypeptide further comprises an amino acid sequence of SEQ ID NO: 100, or a sequence having at least 80%, at least 85%, at least 90%, at least 95%, at least 97% or at least 99% identity thereto; and (b) two polypeptides comprising at least one light chain variable region domain comprising: a LC CDR1 sequence comprising an amino acid sequence of SEQ ID NO: 7; a LC CDR2 sequence comprising an amino acid sequence of SEQ ID NO: 8; a LC CDR3 sequence comprising an amino acid sequence of SEQ ID NO: 9; and wherein the polypeptide further comprises an amino acid sequence of SEQ ID NO: 101, or a sequence having at least 80%, at least 85%, at least 90%, at least 95%, at least 97% or at least 99% identity thereto.

The disclosure provides antibodies or antibody-drug conjugates, comprising a first antigen binding domain that specifically binds to a first 5T4 epitope; a second antigen binding domain that specifically binds to a second 5T4 epitope that is not the same as the first 5T4 epitope; wherein the first antigen binding domain is operably linked to the second antigen binding domain; and a chemotherapeutic agent, wherein the first antigen binding domain comprises a full-length IgG antibody and the second antigen binding domain comprises an scFv, and the antibody or antibody-drug conjugate comprises: (a) two polypeptides comprising at least one heavy chain variable region domain comprising: a HC CDR1 sequence comprising an amino acid sequence of SEQ ID NO: 4; a HC CDR2 sequence comprising an amino acid sequence of SEQ ID NO: 5; a HC CDR3 sequence comprising an amino acid sequence of SEQ ID NO: 6; and wherein the polypeptide further comprises an amino acid sequence of SEQ ID NO: 100, or a sequence having at least 80%, at least 85%, at least 90%, at least 95%, at least 97% or at least 99% identity thereto; and (b) two polypeptides comprising at least one light chain variable region domain comprising: a LC CDR1 sequence comprising an amino acid sequence of SEQ ID NO: 10; a LC CDR2 sequence comprising an amino acid sequence of SEQ ID NO: 11; and a LC CDR3 sequence comprising an amino acid sequence of SEQ ID NO: 12; and wherein the polypeptide further comprises an amino acid sequence of SEQ ID NO: 101, or a sequence having at least 80%, at least 85%, at least 90%, at least 95%, at least 97% or at least 99% identity thereto.

The present disclosure provides an antibody or antibody-drug conjugate, comprising a first antigen binding domain that specifically binds to a first 5T4 epitope; a second antigen binding domain that specifically binds to a second 5T4 epitope that is not the same as the first 5T4 epitope; wherein the first antigen binding domain is operably linked to the second antigen binding domain; and a chemotherapeutic agent, wherein the first antigen binding domain comprises a full-length IgG antibody and the second antigen binding domain comprises an scFv, and the antibody or antibody-drug conjugate comprises: (a) two polypeptides comprising a first heavy chain variable region domain comprising: a HC CDR1 sequence comprising an amino acid sequence of SEQ ID NO: 1; a HC CDR2 sequence comprising an amino acid sequence of SEQ ID NO: 2; a HC CDR3 sequence comprising an amino acid sequence of SEQ ID NO: 3; and an at least second heavy chain variable region domain comprising: a HC CDR1 sequence comprising an amino acid sequence of SEQ ID NO: 4; a HC CDR2 sequence comprising an amino acid sequence of SEQ ID NO: 5; a HC CDR3 sequence comprising an amino acid sequence of SEQ ID NO: 6; and light chain variable region domain comprising: a LC CDR1 sequence comprising an amino acid sequence of SEQ ID NO: 7; a LC CDR2 sequence comprising an amino acid sequence of SEQ ID NO: 8; a LC CDR3 sequence comprising an amino acid sequence of SEQ ID NO: 9; and wherein the polypeptide further comprises an amino acid sequence of SEQ ID NO: 100, or a sequence having at least 80%, at least 85%, at least 90%, at least 95%, at least 97% or at least 99% identity thereto; and (b) two polypeptides comprising a light chain variable region domain comprising: a LC CDR1 sequence comprising an amino acid sequence of SEQ ID NO: 10; a LC CDR2 sequence comprising an amino acid sequence of SEQ ID NO: 11; a LC CDR3 sequence comprising an amino acid sequence of SEQ ID NO: 12; and wherein the polypeptide further comprises an amino acid sequence of SEQ ID NO: 101, or a sequence having at least 80%, at least 85%, at least 90%, at least 95%, at least 97% or at least 99% identity thereto.

The disclosure provides antibodies or antibody-drug conjugates, comprising a first antigen binding domain that specifically binds to a first 5T4 epitope; a second antigen binding domain that specifically binds to a second 5T4 epitope that is not the same as the first 5T4 epitope; wherein the first antigen binding domain is operably linked to the second antigen binding domain; and a chemotherapeutic agent, wherein the first antigen binding domain comprises a full-length IgG antibody and the second antigen binding domain comprises an scFv, and the antibody or antibody-drug conjugate comprises: (a) two polypeptides comprising a first heavy chain variable region domain comprising: a HC CDR1 sequence comprising an amino acid sequence of SEQ ID NO: 1; a HC CDR2 sequence comprising an amino acid sequence of SEQ ID NO: 2; a HC CDR3 sequence comprising an amino acid sequence of SEQ ID NO: 3; and an at least second heavy chain variable region domain comprising: a HC CDR1 sequence comprising an amino acid sequence of SEQ ID NO: 4; a HC CDR2 sequence comprising an amino acid sequence of SEQ ID NO: 5; a HC CDR3 sequence comprising an amino acid sequence of SEQ ID NO: 6; and light chain variable region domain comprising: a LC CDR1 sequence comprising an amino acid sequence of SEQ ID NO: 10; a LC CDR2 sequence comprising an amino acid sequence of SEQ ID NO: 11; a LC CDR3 sequence comprising an amino acid sequence of SEQ ID NO: 12; and wherein the polypeptide further comprises an amino acid sequence of SEQ ID NO: 100, or a sequence having at least 80%, at least 85%, at least 90%, at least 95%, at least 97% or at least 99% identity thereto; and (b) two polypeptides comprising a light chain variable region domain comprising: a LC CDR1 sequence comprising an amino acid sequence of SEQ ID NO: 7; a LC CDR2 sequence comprising an amino acid sequence of SEQ ID NO: 8; a LC CDR3 sequence comprising an amino acid sequence of SEQ ID NO: 9; and wherein the polypeptide further comprises an amino acid sequence of SEQ ID NO: 101, or a sequence having at least 80%, at least 85%, at least 90%, at least 95%, at least 97% or at least 99% identity thereto.

### Therapeutic Agents

The disclosed antigen binding domains and antibodies specific for 5T4 can be conjugated to a therapeutic agent or effector molecule including, but not limited to, molecules in which there is a covalent linkage of the therapeutic agent to the antigen binding domain or antibody. A therapeutic agent is an agent with a particular biological activity directed against a particular target molecule or a cell bearing a target molecule. One of skill in the art will appreciate that therapeutic agents can include various drugs such as vinblastine, daunomycin and the like, cytotoxins such as native or modified Pseudomonas exotoxin or Diphtheria toxin, encapsulating agents (such as liposomes) which themselves contain pharmacological compositions, radioactive agents such as ¹²⁵I, ³²P, ¹⁴C, ³H and ³⁵S and other labels, target moieties and ligands.

In some embodiments, the therapeutic agent is a chemotherapeutic agent.

The choice of a particular therapeutic agent depends on the particular target molecule or cell, and the desired biological effect. Thus, for example, the therapeutic agent can be a cytotoxin that is used to bring about the death of a particular target cell (such as a tumor cell). Conversely, where it is desired to invoke a non-lethal biological response, the therapeutic agent can be conjugated to a non-lethal pharmacological agent or a liposome containing a non-lethal pharmacological agent.

Effector molecules can be linked to an antigen binding domain or antibody of interest using any number of means known to those of skill in the art. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antigen binding domain or antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the protein and to the effector molecule. Suitable linkers are well known to those of skill in the art and include, but are not limited to, straight or branched-chain carbon linkers, heterocyclic carbon linkers, or peptide linkers. Where the protein and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antigen binding domain or antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

The person skilled in the art will be able to determine a suitable method for attaching a given agent to an antibody or other polypeptide.

In some aspects, the chemotherapeutic agent is conjugated to at least one of the first antigen binding domain or second binding domain of the biparatopic antibodies described herein via a linker. In some aspects, the chemotherapeutic agent is an auristatin. In some aspects, the auristatin is selected from the group consisting of auristatin E (AE), monomethyl auristatin D (MMAD), monomethyl auristatin E (MMAE), monomethyl auristatin F (MMAF), and synthetic analogs of dolastatin. In some aspects the linker is a cleavable linker. In some aspects, the linker is a non-cleavable linker.

The anti-5T4 biparatopic antibody-drug conjugates (ADC) of the present disclosure comprises a first antigen binding domain that specifically binds to a first 5T4 epitope, a second antigen binding domain that specifically binds to a second 5T4 epitope that is not the same as the first 5T4 epitope, wherein the first antigen binding domain is operably linked to the second antigen binding domain, and is conjugated to a cytotoxic or immunosuppressive agent, such that the resulting ADC exerts a cytotoxic or cytostatic effect on an 5T4-expressing cancer cell. Thus, the anti-5T4 biparatopic antibody-drug conjugate exerts a cytotoxic or cytostatic effect on 5T4-expressing cancer cells. In one embodiment, the anti-5T4 ADC is internalized and accumulates within a 5T4-expressing cell, where the ADC exerts a therapeutic effect (e.g., a cytotoxic, cytostatic, or immunosuppressive effect).

Examples of suitable moieties for conjugation to antigen binding domains and antibodies include chemotherapeutic agents, prodrug converting enzymes, radioactive isotopes or compounds, or toxins. In exemplary embodiments, the anti-5T4 antibody, or an antigen-binding portion thereof is conjugated to an auristatin, e.g., MMAF or MMAE. Any agent that exerts a therapeutic effect on cancer cells or activated immune cells can be used as the therapeutic agent for conjugation to an anti-5T4 antibody or derivative thereof (See, e.g., WO 2004/010957, “Drug Conjugates and Their Use for Treating Cancer, An Autoimmune. Disease or an Infectious Disease” (supra) and U.S. Provisional Application No. 60/400,403 (supra)). Typically, the therapeutic agent is a cytotoxic agent. In some embodiments, an anti-5T4 antibody-drug conjugate comprises more than one therapeutic agents per conjugate, for example from about 1 to about 20 therapeutic agents per conjugate (commonly referred to as the drug to antibody ratio, or DAR).

In some embodiments, the anti-5T4 antibody, or an antigen-binding portion thereof, is conjugated to an auristatin. Auristatins have been shown to interfere with microtubule dynamics, GTP hydrolysis, and/or nuclear and cellular division and have anticancer and/or antifungal activity.

An anti-5T4 antibody or antigen binding domain of the disclosure may be conjugated to at least one auristatin. Auristatins represent a group of dolastatin analogs that have generally been shown to possess anticancer activity by interfering with microtubule dynamics and GTP hydrolysis, thereby inhibiting cellular division. For example, Auristatin E (described in U.S. Pat. No. 5,635,483, incorporated by reference herein) is a synthetic analogue of the marine natural product dolastatin 10, a compound that inhibits tubulin polymerization by binding to the same site on tubulin as the anticancer drug vincristine (G. R. Pettit, Prog. Chem. Org. Nat. Prod, 70: 1-79 (1997)). Dolastatin 10, auristatin PE, and auristatin E are linear peptides having four amino acids, three of which are unique to the dolastatin class of compounds. Exemplary embodiments of the auristatin subclass of mitotic inhibitors include, but are not limited to, monomethyl auristatin D (MMAD or auristatin D derivative), monomethyl auristatin E (MMAE or auristatin E derivative), monomethyl auristatin F (MMAF or auristatin F derivative), auristatin F phenylenediamine (AFP), auristatin EB (AEB), auristatin EFP (AEFP), and 5-benzoylvaleric acid-AE ester (AEVB). The synthesis and structure of auristatin derivatives are described in U.S. Patent Application Publication Nos. 2003-0083263, 2005-0238649 and 2005-0009751; International Patent Publication No. WO 04/010957, International Patent Publication No. WO 02/088172, and U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein.

In some embodiments, an anti-5T4 antibody or antigen binding domain is conjugated to at least one MMAF (monomethyl auristatin F) by a linker such as, but not limited to, maleimidocaproyl (mc-MMAF). An anti-5T4 ADC may have a drug-to-antibody ratio (DAR) of 2, 4, 6, or 8. Notably, the DAR of an ADC can range from 0 to 8, although higher loads, e.g., 10, 12 or 14 are also possible. Monomethyl auristatin F (MMAF) inhibits cell division by blocking the polymerization of tubulin. It has a charged C-terminal phenylalanine residue that attenuates its cytotoxic activity compared to its uncharged counterpart MMAE. Because of its toxicity, it cannot be used as a drug itself, but can be linked to a monoclonal antibody (mAb) that directs it to the cancer cells. In one embodiment, the linker to the anti-5T4 antibody is stable in extracellular fluid but is cleaved by cathepsin once the conjugate has entered a tumor cell, thus activating the anti-mitotic mechanism.

In some embodiments, the anti-5T4 antibody or antigen binding domain of the invention is conjugated to at least one MMAE (mono-methyl auristatin E). Monomethyl auristatin E (MMAE, vedotin) inhibits cell division by blocking the polymerisation of tubulin. Because of its toxicity, it frequently cannot be used as a drug itself. In recent cancer therapy developments, it is linked to a monoclonal antibody (mAb) that recognizes a specific marker expression in cancer cells and directs MMAE to the cancer cells. In some embodiments, the linker linking MMAE to the anti-5T4 antibody or antigen binding domain is stable in extracellular fluid (i.e., the medium or environment that is external to cells) but is cleaved by cathepsin once the ADC has bound to the specific cancer cell antigen and entered the cancer cell, thus releasing the toxic MMAE and activating the potent anti-mitotic mechanism.

In some embodiments, the anti-5T4 antibody, or antigen-binding portion thereof, is conjugated to an auristatin which is MMAF. In some embodiments, the anti-5T4 ADC is covalently linked to one or more molecules of monomethyl auristatin F (MMAF). In some embodiments, to generate the anti-5T4 ADC covalently linked to one or more molecules of MMAF, the interchain disulfide bonds of the ADC are reduced to sulfhydryl groups. MMAF is then coupled to the antibody via these sulfhydryl groups. In some embodiments, the anti-5T4 ADC is generated using a noncleavable linker, i.e., a noncleavable maleimidocaproyl (mc) linkage.

ADCs that can be labeled with a detectable or functional label. Detectable labels include, but are not limited to, radiolabels such as the isotopes .sup.2H, .sup.3H, .sup.11C, .sup.13C, .sup.14C, .sup.32P, .sup.33S, .sup.34S, .sup.35S, .sup.36S, .sup.36C1, .sup.51Cr, .sup.57Co, .sup.58Co, .sup.59Fe, .sup.90Y, .sup.1211, .sup.1241, .sup.1251, .sup.1311, .sup.211At, .sup.198Au, .sup.67Cu, .sup.225Ac, .sup.213Bi, sup.99Tc and .sup.186Re, which may be attached to antibodies of the invention using conventional chemistry known in the art of antibody imaging. Labels also include fluorescent labels and labels used conventionally in the art for MRI-CT imagine. They also include enzyme labels such as horseradish peroxidase. Labels further include chemical moieties such as biotin which may be detected via binding to a specific cognate detectable moiety, e.g., labeled avidin.

Functional labels may also include substances which are designed to be targeted to the site of a tumor to cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

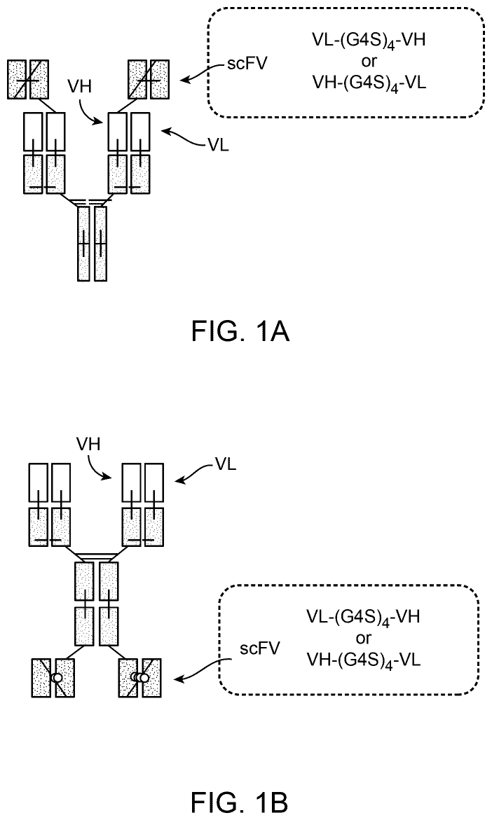

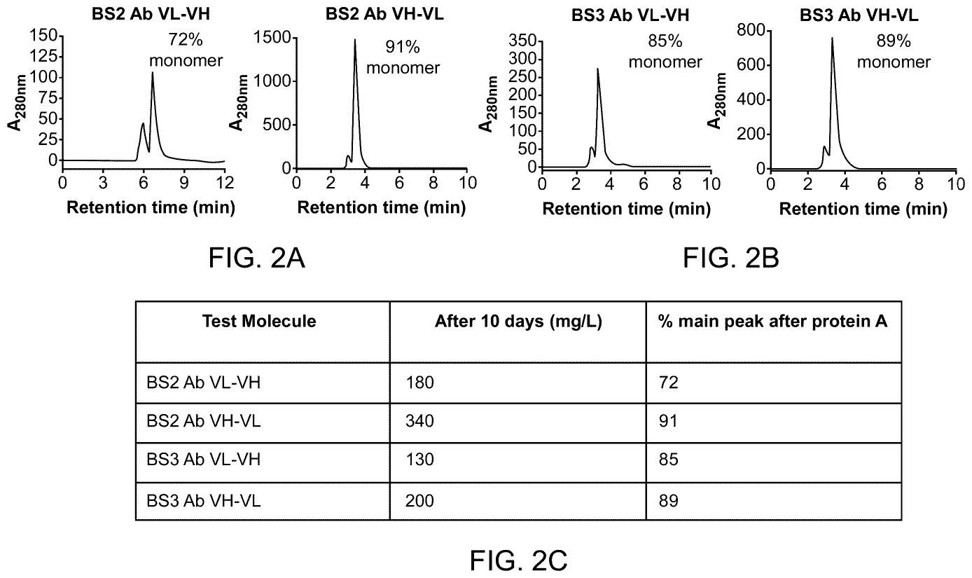

As will be understood by those of skill in the art, the agents set forth above, as well as other suitable agents, may be conjugated or attached to an anti-5T4 antibody, such as the antibodies depicted in FIGS. 1A-1B, in any suitable manner to produce an anti-5T4 ADC of the disclosure. For example, and without limitation, in various embodiments of the present disclosure the anti-5T4 antibody and agent(s) may be covalently attached and/or may be conjugated using linker, spacer and/or stretcher compounds, which in various embodiments of the present invention are cleavable or are noncleavable, and result in the therapeutic agent(s) being internalized by the target cell.

In one embodiment, the anti-5T4 antibody or antigen binding domain is conjugated to MMAF using a noncleavable maleimidocaproyl linkage.

Techniques for conjugating therapeutic agents to proteins, and in particular to antibodies, are known in the art (see, e.g., Amon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Dekker, Inc., 2nd ed. 1987); Thorpe, “Antibody Carriers Of Cytotoxic Agents In Cancer Therapy: A Review,” in Monoclonal Antibodies'84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624).

In some embodiments, the ADC comprises a linker region between the cytotoxic agent and the antibody or antigen binding domain. For example, such linker, spacer and/or stretcher compounds include, but are not limited to, the following: amino benzoic acid spacers (see, for example and without limitation, U.S. Pat. Nos. 7,091,186 and 7,553,816, each of which is hereby incorporated by reference in its entirety); maleimidocaproyl; p-aminobenzylcarbamoyl (PAB); lysosomal enzyme-cleavable linkers (see, for example and without limitation, U.S. Pat. No. 6,214,345, hereby incorporated by reference in its entirety); maleimidocaproyl-polyethylene 20 glycol (MC(PEG)₆-OH); N-methyl-valine citrulline; N-succinimidyl 4-(N-maleimidomethyl) cyclohexane-1-carboxylate (SMCC) (see, for example and without limitation, Yoshitake et al. (1979) Eur. J. Biochem., 101, 395-399, hereby incorporated by reference in its entirety); N-succinimidyl 4-(2-pyridyldithio) butanoate (SPDB) (see, for example and without limitation, U.S. Pat. No. 4,563,304, hereby incorporated by reference 25 in its entirety); N-Succinimidyl 4-(2-pyridylthio)pentanoate (SPP); valine-citrulline; and other linker, spacer, and/or stretcher compounds (see, for example and without limitation, U.S. Pat. Nos. 7,090,843, 7,223,837, and 7,659,241, and U.S. Patent Publication Nos. 2004/0018194, 2004/0121940, 2006/0116422, 2007/0258987, 2008/0213289, 2008/0241128, 2008/0311136, 2008/0317747, and 2009/0010945, each of which is hereby incorporated by reference in its entirety). Generally speaking, techniques for attaching and/or conjugating the agents set forth above, as well as other agents, to specific binding members of the present invention, particularly antibodies and fragments thereof, are known in the art. See, for example and without limitation, Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, In Monoclonal Antibodies'84: Biological And Clinical Applications, Pinchera et al. (eds.), pp. 475-506 (1985), each of which is hereby incorporated by reference in its entirety.

A number of different reactions are available for covalent attachment of drugs to antibodies or antigen binding domains. This is often accomplished by reaction of the amino acid residues of the antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of the antibody molecule. Also available for attachment of drugs to antibodies is the Schiff base reaction. This method involves the periodate oxidation of a drug that contains glycol or hydroxy groups, thus forming an aldehyde which is then reacted with the antibody molecule. Attachment occurs via formation of a Schiff base with amino groups of the antibody molecule. Isothiocyanates can also be used as coupling agents for covalently attaching drugs to antibodies. Other techniques are known to the skilled artisan and within the scope of the present invention. Non-limiting examples of such techniques are described in, e.g., U.S. Pat. Nos. 5,665,358; 5,643,573; and 5,556,623, which are incorporated by reference in their entireties herein.

In certain embodiments, an intermediate, which is the precursor of the linker, is reacted with the drug under appropriate conditions. In certain embodiments, reactive groups are used on the drug and/or the intermediate. The product of the reaction between the drug and the intermediate, or the derivatized drug, is subsequently reacted with the anti-5T4 antibody under appropriate conditions.

Other examples of conjugation methods are described in U.S. Pat. No. 7,837,980 (Seattle Genetics), Carter and Senter (2008) Cancer J, 14(3):154, as well as U.S. Published Application Nos. 2004-0157782 A1 and 2005-0238649 and International Patent Application No, PCT/US04/038392.

In certain embodiments, anti-5T4 ADCs may be purified in order to obtain ADCs having a desired drug to antibody ration (DAR). In one embodiment of the invention, the formulation contains an anti-5T4 ADC mixture comprising anti-5T4 ADCs having an average desired drug to antibody ration (DAR), e.g., an average DAR of about 3. In one embodiment of the invention, the formulation comprises an ADC mixture comprising anti-5T4 ADCs having a desired DAR range, e.g., a DAR of about 2-4, or about 2-8, or about 4-8.

In one embodiment, the formulation contains an ADC mixture where 70% of the ADCs present have a drug loaded species of 8 or less, and wherein the ADC comprises an anti-5T4 antibody and an auristatin. Alternatively, 75% of ADCs present have a drug loaded species of 8 or less; 80% of ADCs present have a drug loaded species of 8 or less; 85% of ADCs present have a drug loaded species of 4 or less; 90% of ADCs present have a drug loaded species of 8 or less; or 95% of ADCs present have a drug loaded species of 8 or less.

### Polynucleotides and Vectors

The disclosure provides polynucleotides, and polynucleotide systems comprising one or more polynucleotides, encoding the anti-5T4 antibodies, antigen binding domains, and receptors described herein.

In some embodiments, the polynucleotides or polynucleotide systems described herein comprise sequences encoding any of the light chain CDR sequences and/or heavy chain CDR sequences of the anti-5T4 biparatopic antibodies described herein.

In some embodiments, the polynucleotides or polynucleotide systems described herein comprise sequences encoding any of the light chains and heavy chains of the anti-5T4 biparatopic antibodies described herein.

In some embodiments, the polynucleotides or polynucleotide systems described herein comprise sequences as set forth in any of Tables 4-7.

The disclosure provides a first polynucleotide encoding a first heavy chain of an anti-5T4 antigen binding domain disclosure, and a second polynucleotide encoding a light chain of an anti-5T4 antigen binding domain. In some embodiments, the first and second polynucleotides are separate molecules. Alternatively, the first and second polynucleotides may form part of a single contiguous polynucleotide molecule.

In some embodiments, the disclosure provides a first polynucleotide encoding a first anti-5T4 antigen binding domain heavy chain, a linker, and a second anti-5T4 antigen binding domain; and a second a polynucleotide encoding a first anti-5T4 antigen binding domain light chain. In some embodiments, the disclosure provides a single contiguous polynucleotide molecule encoding a first anti-5T4 antigen binding domain heavy chain, a first anti-5T4 antigen binding domain light chain, and a second anti-5T4 antigen binding domain.

In some embodiments, the first polynucleotide encodes a sequence of a first polypeptide comprising, from N to C terminus, a first anti-5T4 antigen binding domain heavy chain, a linker, and a second anti-5T4 antigen binding domain. In some embodiments, the first polynucleotide encodes a sequence of a first polypeptide comprising, from N to C terminus, a second anti-5T4 antigen binding domain, a linker and a first anti-5T4 antigen binding domain heavy chain. In some embodiments, the first polynucleotide encodes a sequence of a first polypeptide, comprising, from N to C terminus, a second anti-5T4 antigen binding domain, a linker and a first anti-5T4 antigen binding domain heavy chain. In some embodiments, the first polynucleotide comprises a nucleotide sequence of SEQ ID NO: 144 or 145. In some embodiments, the first polynucleotide comprises a nucleotide sequence of SEQ ID NO: 144 and 150. In some embodiments, the first polynucleotide comprises a nucleotide sequence of SEQ ID NO: 145 and 150. In some embodiments, the first polynucleotide comprises a nucleotide sequence of SEQ ID NO: 142.

In some embodiments, the second polynucleotide encodes a second polypeptide comprising a first anti-5T4 antigen binding domain light chain. In some embodiments, the second polynucleotide comprise a nucleotide sequence of SEQ ID NO: 146 or 147. In some embodiments, the second polynucleotide comprise nucleotide sequence of SEQ ID NO: 146 and 151. In some embodiments, the second polynucleotide comprise a nucleotide sequence of SEQ ID NO: 147 and 151. In some embodiments, the second polynucleotide comprises a nucleotide sequence of SEQ ID NO: 143.

In some embodiments, the first polynucleotide encodes a polypeptide comprising a sequence of SEQ ID NO: 100, or a sequence having at least 80%, at least 85%, at least 90%, at least 95%, at least 97% or at least 99% identity thereto. In some embodiments, the second polynucleotide encode a polypeptide comprising a sequence of SEQ ID NO: 101, or a sequence having at least 80%, at least 85%, at least 90%, at least 95%, at least 97% or at least 99% identity thereto.

In some embodiments, the polynucleotide sequences encoding each of the first, and second polypeptides are operably linked to one or more promoters. For example, sequences of the polypeptides can be operably linked (under the control of) the same promoter, and separated by one or more elements that produce separate polypeptides, such as self-cleaving polypeptides, internal ribosome entry sites, and the like.

In alternative embodiments, the sequences of the first or second polynucleotides encoding the first or second polypeptides are under the control of separate promoters. For example, each of the first, and second polynucleotides may be cloned into a separate expression vector, each vector comprising its own promoter and/or regulatory sequences. In some embodiments, the promoters operably linked to each of the first and second polynucleotides are the same. In some embodiments, the promoters operably linked to each of the first and second polynucleotides are not the same.

In some embodiments, polynucleotides of the present invention are prepared using PCR techniques using procedures and methods known to one skilled in the art. In some embodiments, the procedure involves the ligation of two different DNA sequences (See, for example, “Current Protocols in Molecular Biology”, eds. Ausubel et al., John Wiley & Sons, 1992).

With the antigen binding domains, antibodies and receptors described herein, one of skill can readily construct a variety of clones containing functionally equivalent nucleic acids, such as nucleic acids which differ in sequence but which encode the same protein sequence. Thus, the present disclosure provides nucleic acids encoding antibodies thereof.

A polynucleotide sequence is “operably linked” when it is placed into a functional relationship with another polynucleotide sequence. For example, a polynucleotide presequence or secretory leader is operably linked to a nucleic acid encoding a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the polynucleotide sequences being linked are contiguous, and, in the case of a secretory leader, contiguous and in reading frame. However, enhancers are optionally contiguous. Linking can be accomplished, for example, by ligation at convenient restriction sites. If such sites do not exist, synthetic oligonucleotide adaptors, linkers or other methods known in the art can be used. In another embodiment, “operably linked” also refers to the functional pairing of distinct amino acid sequences, peptides or proteins, as in the combination of the first anti-5T4 antigen binding domain heavy chain and second anti-5T4 antigen binding domain described herein, which are operably linked, optionally via linker sequences also described herein. For example, the first anti-5T4 antigen binding domain heavy chain is “operably linked” to the second anti-5T4 antigen binding domain via a linker sequence as described herein.

The disclosure provides vectors comprising the polynucleotides comprising sequences encoding the 5T4 antigen binding domains, antibodies and receptors described herein.

The terms “vector”, “cloning vector” and “expression vector” mean the vehicle by which a DNA or RNA sequence (e.g., a foreign gene) can be introduced into a host cell, so as to transform the host and promote expression (e.g. transcription and translation) of the introduced sequence. Vectors include plasmids, phages, viruses, etc.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane. The term “intracellular” means something that is inside a cell. The term “extracellular” means something that is outside a cell. The term transmembrane means something that has an extracellular domain outside the cell, a portion embedded in the cell membrane and an intracellular domain inside the cell.

In some embodiments polynucleotides of the present disclosure are inserted into expression vectors (i.e., a nucleic acid construct) to enable expression of the polypeptides described herein.

In some embodiment, the expression vector of the present disclosure includes additional sequences which render this vector suitable for replication and integration in prokaryotes. In some embodiments, the expression vector of the present disclosure includes additional sequences which render this vector suitable for replication and integration in eukaryotes. In some embodiments, the expression vector of the present disclosure includes a shuttle vector which renders this vector suitable for replication and integration in both prokaryotes and eukaryotes. For example, such vectors may include selectable markers appropriate for both eukaryotic and prokaryotic cells. Suitable markers will be apparent to persons of ordinary skill in the art.

In some embodiments, cloning vectors comprise transcription and translation initiation sequences (e.g., promoters, enhancer) and transcription and translation terminators (e.g., polyadenylation signals) to enhance expression of polypeptides expressed therefrom. Suitable translation terminators include, but are not limited, to bovine growth hormone polyadenylation signals (BGH polyA) and the like. Suitable promoters will be apparent to persons of the ordinary skill in the art, and include the CMV promoter, actin promoter and the like.

In some embodiments, the expression vectors of the present disclosure can further include additional polynucleotide sequences that allow, for example, the translation of several proteins from a single mRNA such as an internal ribosome entry site (IRES) and sequences for genomic integration of the promoter-chimeric polypeptide.

In some embodiments, the expression vectors of the present disclosure include elements that increase the expression of the antibodies of the invention. Such features include, but are not limited to, choice of promoter and polyadenylation. In some embodiments, the polyadenylation sequence is a bovine growth hormone (BGH) polyadenylation sequence. In some embodiments, the promoter comprises a constitutively active promoter. In some embodiments, the promoter comprises a cytomegalovirus promoter (pCMV). Promoters can, in some embodiments, be combined with additional elements to promote expression of the recombinant proteins of the disclosure, such as introns (e.g., rabbit beta globin intron, EF1a intron and the like) and enhancer elements (CMV immediate early enhancer, SV40 enhancer, EF1a enhancer, adenoviral major late protein enhancer, and the like).

Exemplary mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Barr virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, for example in bacterial systems used to express the polypeptides of the present invention, a number of expression vectors can be advantageously selected depending upon the use intended for the protein expressed. In some embodiments, vectors that direct the expression of high levels of the protein product, possibly as a fusion with a hydrophobic signal sequence, which directs the expressed product into the periplasm of the bacteria or the culture medium where the protein product is readily purified are desired. In one embodiment, vectors adaptable to such manipulation include, but are not limited to, the pET series of E. coli expression vectors (see Studier et al., Methods in Enzymol. 185:60-89 (1990)).

In some embodiments, yeast expression systems are used to express the polypeptides of the disclosure. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In some embodiments, recombinant viral vectors are useful for in vivo expression of the polypeptides of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In some embodiments, mammalian cell expression systems are used to express the polypeptides of the disclosure. The mammalian cells can be, for example Chinese Hamster Ovary (CHO) cells or derivatives thereof, and the vector is a vector suitable for expression of the polypeptides in CHO cells. In some embodiments, the mammalian cells can be ExpiCHO-S™ cells.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

### Methods of Manufacture

The disclosure provides methods of making the anti-5T4 antibodies described herein comprising: (a) contacting a plurality of cells with polynucleotides, polynucleotide systems or vectors encoding the antibody; (b) culturing the plurality of cells under conditions whereby the antibody is expressed by at least one cell of the plurality of cells; and (c) purifying the antibody.

The disclosure provides methods of making the anti-5T4 antibody-drug conjugates described herein comprising: (a) contacting a plurality of cells with polynucleotides, polynucleotide systems or vectors encoding the antibody; (b) culturing the plurality of cells under conditions whereby the bispecific antibody is expressed by at least one cell of the plurality of cells; (c) purifying the antibody; and (d) conjugating the antibody to a chemotherapeutic agent. In some embodiments, the antibody is bispecific or biparatopic, as described herein.

The disclosure provides methods of manufacturing an antibody-drug conjugate described herein comprising: (a) culturing a cell comprising a nucleic acid construct that encodes the bispecific or biparatopic antibody under conditions that lead to expression of the bispecific or biparatopic antibody, (b) recovering the bispecific or biparatopic antibody, and (c) conjugating the bispecific or biparatopic antibody to a chemotherapeutic agent.

The disclosure provides methods of manufacturing the antibodies described herein comprising: (a) culturing a cell comprising a nucleic acid construct that encodes the antibody under conditions that lead to expression of the antibody, wherein the antibody comprises the following structure: i) a first antibody that specifically binds to a first 5T4 epitope; ii) a second antibody that specifically binds to a second 5T4 epitope that is not the same as the first 5T4 epitope; wherein the first antibody is operably linked to the second antibody; and (b) recovering the bispecific antibody.

A variety of prokaryotic or eukaryotic cells can be used as host-expression systems to express the antibodies of the present invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence.

In some embodiments, the plurality of cells comprises eukaryotic cells. In some embodiments, the eukaryotic cells are mammalian cells. Mammalian cells suitable for expression of antibody-drug conjugates include CHO cells, PER.C6 cells, murine NS0 cells, and HEK293 cells. Selection of a suitable cell line will be apparent to persons of ordinary skill in the art.

In some embodiments, the plurality of cells comprises prokaryotic cells, for example E. coli cells.

Various methods can be used to introduce the expression vector encoding the antibody of the present disclosure into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, contacting the plurality of cells with the polynucleotides or vectors encoding the antibodies of the disclosure comprises transfection.

The term “transfection” means the introduction of a foreign nucleic acid into a cell using recombinant DNA technology. The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

In some embodiments, contacting the plurality of cells with the polynucleotides or vectors encoding the antibodies of the disclosure comprises transduction. The term “transduction” means the introduction of a foreign nucleic acid into a cell using a viral vector, such as a lentiviral vector.

In some embodiments, non-bacterial expression systems are used (e.g., mammalian expression systems such as CHO cells) to express the antibody polypeptides. In some embodiments, the expression vector comprises a CMV promoter and a neomycin resistance gene. In alternative embodiments, the expression vector comprises a glutamine synthase marker (GS) under control of an SV40 promoter.

In some embodiments, introduction of nucleic acid by viral infection offers several advantages over other methods such as lipofection and electroporation since higher transfection efficiency can be obtained due to the infectious nature of viruses.

In some embodiments, transformed cells are cultured under effective conditions, which allow for the expression of high amounts of antibody or polypeptide. In some embodiments, effective culture conditions include, but are not limited to, effective media, bioreactor, temperature, pH and oxygen conditions that permit protein production. A medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. Culturing conditions are within the expertise of one of ordinary skill in the art.

For example, appropriate media for the culture of eukaryotic cells includes, but is not limited to, Iscove's Modified Dulbecco's Medium, RPMI 1640, Minimal Essential Medium-alpha (MEM-alpha), Dulbecco's Modification of Eagle's Medium (DMEM), Grace's Complete Insect Medium, Ham's F-10 or F-12 with L-Glutamine, Schneider's Insect Medium, or any other media known to one skilled in the art. Additionally, culture media as described herein include, but are not limited to, chemically defined media, hydrolysate-containing media, and simple media. Choice of appropriate media and cell culture conditions for a particular cell type will be apparent to the person of ordinary skill in the art.

In some embodiments, depending on the vector and host system used for production, resultant polypeptides of the present invention either remain within the recombinant cell, secreted into the fermentation medium, secreted into a space between two cellular membranes, such as the periplasmic space in E. coli; or retained on the outer surface of a cell or viral membrane.

In some embodiments, following a predetermined time in culture, the antibody or polypeptide is recovered.

Polypeptides of the present invention are purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In some embodiments, to facilitate recovery, the expressed coding sequence can be engineered to encode the polypeptide of the present invention and fused cleavable moiety. For example, a polypeptide can be designed so that the polypeptide can be readily isolated by affinity chromatography; e.g., by immobilization on a column specific for the cleavable moiety. In one embodiment, a cleavage site is engineered between the polypeptide and the cleavable moiety and the polypeptide can be released from the chromatographic column by treatment with an appropriate enzyme or agent that specifically cleaves the polypeptide at this site [e.g., see Booth et al., Immunol. Lett. 19:65-70 (1988); and Gardella et al., J. Biol. Chem. 265:15854-15859 (1990)].

In some embodiments, the polypeptide of the present invention is retrieved in “substantially pure” form. The phrase “substantially pure” refers to a purity that allows for the effective use of the protein in the applications described herein.

In some embodiments, the polypeptides of the present invention can also be synthesized using in vitro expression systems. In one embodiment, in vitro synthesis methods are well known in the art and the components of the system are commercially available.

In some embodiments, the polypeptides are synthesized and purified; and their therapeutic efficacy is assayed in vivo or in vitro.

### Pharmaceutical Compositions

The disclosure provides pharmaceutical compositions comprising anti-5T4 antigen binding domains, antibodies, and bispecific or biparatopic antibody-drug conjugates described herein, and a pharmaceutically acceptable carrier, diluent or excipient. Pharmaceutical compositions comprising immune cells comprising CARs comprising the antigen binding domains described herein are also contemplated as within the scope of the instant disclosure.

As used herein, “pharmaceutical carrier” includes any and all solvents, dispersion media, coatings, antibacterial and antifungal agents, isotonic and absorption delaying agents, and the like that are physiologically compatible. Carrier materials are non-toxic and do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. The term “carrier” denotes an organic or inorganic ingredient, natural or synthetic, with which the active ingredient is combined to facilitate the application. Preferably, the carrier is suitable for intravenous, intramuscular, subcutaneous, parenteral, spinal or epidermal administration (e.g., by injection or infusion).

Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The pharmaceutical compositions may be present in a form known in the art and acceptable for therapeutic uses. In some embodiments, pharmaceutical compositions of the invention is a liquid formulation. In other embodiments, pharmaceutical compositions of the invention are lyophilized. In further embodiments, pharmaceutical compositions of the invention are reconstituted liquid formulations. In some embodiments, a liquid formulation of the invention is an aqueous formulation. In other embodiments, the liquid formulation is non-aqueous.

Compositions comprising the anti-5T4 antigen binding domains, antibodies and bispecific antibody-drug conjugates of the present disclosure can be formulated for administration by a variety of methods known in the art. As will be appreciated by the person of ordinary skill in the art, the route and/or mode of administration will vary depending upon the desired results. To administer a composition of the disclosure by certain routes of administration, it may be necessary to co-administer the composition with a material to prevent its inactivation. For example, the antibody-drug conjugate may be administered to a subject in an appropriate carrier, for example, liposomes, or a diluent.

In some embodiments, preparations for administration to subjects include sterile aqueous or non-aqueous solutions, suspensions, and emulsions. Some embodiments include non-aqueous solvents such as propylene glycol, polyethylene glycol, vegetable oils (e.g., olive oils), organic esters (e.g., ethyl oleate) and other solvents known to those of skill in the art. Physiologically acceptable carriers (or excipients) are optionally used in certain embodiments of the invention. Examples of such include, e.g., saline, PBS, Ringer's solution, lactated Ringer's solution, etc. Additionally, preservatives and additives are optionally added to the compositions to help ensure stability and sterility. For example, antibiotics and other bacteriocides, antioxidants, chelating agents, and the like are all optionally present in various embodiments of the compositions herein.

Regardless of the route of administration selected, the compositions of the disclosure, which may be used in a suitable hydrated form, and/or the pharmaceutical compositions of the present invention, are formulated into pharmaceutically acceptable dosage forms by conventional methods known to those of ordinary skill in the art.

The pharmaceutical compositions are optionally administered to subjects in need of treatment (either therapeutically or prophylactically) in any appropriate sterile pharmaceutical carrier. Such pharmaceutical carrier acts to maintain the solubility and action of the anti-5T4 antibody or antibody-drug conjugate.

In some embodiments, compositions for use in the methods disclosed herein comprise solutions or emulsions, which in some embodiments are aqueous solutions or emulsions comprising a safe and effective amount of the compounds disclosed herein and optionally, other compounds, intended for various routes of administration.

The composition must be sterile and fluid to the extent that the composition is deliverable by syringe. In addition to water, the carrier preferably is an isotonic buffered saline solution. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

Actual dosage levels of the active ingredients in the pharmaceutical compositions of the present invention may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts.

### Therapeutic Methods

The disclosure provides methods of treating a disease or disorder in a subject in need thereof, the method comprising administering a therapeutically effective amount of the anti-5T4 bispecific antibody-drug conjugates or the pharmaceutical compositions comprising the bispecific antibody-drug conjugates disclosed herein. Methods comprising administering the anti-5T4 antigen binding domains, e.g. as part of an immunotherapy, are also contemplated as within the scope of the instant disclosure. Methods of the disclosure also include adoptive cell therapies comprising administering immune cells, for example T cells or NK cells, expressing receptors comprising the 5T4 antigen binding domains described herein.

In some embodiments, the disease or disorder is cancer. In some embodiments, the cancer comprises a solid tumor.

In some embodiments, the cancer comprises a solid tumor. In some embodiments, the cancer is selected from the group consisting of melanoma, renal cell carcinoma, mesothelioma, small cell lung cancer, uveal melanoma, bladder cancer, gastric cancer, squamous cell carcinoma of the head and neck, cutaneous carcinoma, non-small cell lung cancer, colorectal cancer, prostate cancer, ovarian cancer, cervical cancer, endometrial carcinoma, breast cancer, pancreatic cancer, urothelial cancer, esophageal cancer, hepatocellular carcinoma, glioblastoma, glioma, or sarcoma.

In some embodiments, the cancer is selected from the group consisting of adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstroem macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, epithelioid sarcoma, synovial sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A cancer treated with the antigen binding domains, antibodies, immune cells, or antibody-drug conjugates or pharmaceutical compositions comprising same of the disclosure can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1 (mi), PN1a, PN1b, PN1c, pN2, pN2a, pN2b, pN3, pN3a, pN3b, or pN3c. Alternatively, or in addition, a cancer can be staged according to the TNM staging system, which divides most types of cancers into 4 stages. Stage 1 usually means that a cancer is relatively small and contained within the organ of origin. Stage 2 cancers have usually not started to spread into surround tissues, but that the tumor is larger than stage 1. In some embodiments, stage 2 means that the cancer has spread into the lymph nodes close to the tumor. Stage 3 cancers are usually larger and have started to spread into surrounding tissues and lymph nodes. Stage 4, or metastatic cancers, are typically cancers that have spread from the point of origin to other organ(s) in the body.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which an antibody-drug conjugate or other composition of matter is in direct contact with a cell, or is close enough to induce a desired biological effect in a cell.

As used herein, “monotherapy” refers to the administration of a single active or therapeutic compound to a subject in need thereof. Preferably, monotherapy will involve administration of a therapeutically effective amount of an active compound. Monotherapy may be contrasted with combination therapy, in which a combination of multiple active compounds is administered, preferably with each component of the combination present in a therapeutically effective amount.

As used herein, “treating” or “treat” describes the management and care of a subject for the purpose of combating a disease, condition, or disorder and includes the administration of an anti-5T4 antigen binding domain, antibody, antibody-drug conjugate, immune cell expressing an anti-5T4 receptor, or pharmaceutical composition comprising same of the disclosure to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of recombinant of anti-5T4 bispecific antibody-drug conjugate or pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

As used herein, the term “aggressive” indicates a cancer that can grow, form or spread quickly. Cancers termed aggressive may be susceptible to treatment, or they may resist treatment. An aggressive cancer can comprise any sort of cancer. Alternatively, or in addition, the term “aggressive” may describe a cancer that requires a more severe or intense than the usual form of treatment for that cancer.

As used herein, the term “refractory” describes a cancer that does not respond to an attempted form of treatment. Refractory cancers can also be termed resistant cancers.

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population that is not receiving the anti-5T4 bispecific antibody-drug conjugate, or pharmaceutical composition comprising same, of the disclosure. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population that is not receiving the anti-5T4 bispecific antibody-drug conjugate, or pharmaceutical composition comprising same, of the disclosure. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not an anti-5T4 bispecific antibody-drug conjugate or pharmaceutical composition of the disclosure. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with the antibody-drug conjugates described herein.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating cancer can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating cancer can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating cancer can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating cancer can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

Treating cancer can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

### Combination Therapies

In some embodiments, it may be desired to administer additional cancer treatments in conjunction with the antigen binding domains, antibodies, antibody-drug conjugates, adoptive cell therapies or pharmaceutical compositions comprising same. For example, in some treatment regimes, chemotherapeutic agents, antibiotics, additional formulations comprising the antibody-drug conjugate of the disclosure and one or more standard of care agents, etc. are all optionally included with the compositions of the invention. In some embodiments, the antibody-drug conjugate is administered in combination with one or more of chemotherapy, a small molecule inhibitor, radiation, surgery, immunotherapy or adoptive cell therapy.

As used herein, the terms “combination treatment,” “combination therapy,” and “co-therapy” are used interchangeably and generally refer to treatment modalities featuring an antibody-drug conjugate or pharmaceutical composition comprising the same as provided herein and an additional therapeutic agent or method. Typically, combination treatment modalities are part of a specific treatment regimen intended to provide a beneficial effect from the concurrent action of the therapeutic agent combination. The beneficial effect of the combination may include, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). In some embodiments, combination treatment comprises administration of two or more therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, separate dosage forms for the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. The therapeutic agents can be administered according to the same or to a different administration interval. For example, a first therapeutic agent of the combination selected may be administered by intravenous injection while the other therapeutic agents of the combination may be administered orally. Alternatively, for example, all therapeutic agents may be administered orally, or all therapeutic agents may be administered by intravenous injection.

In some embodiments, combination therapy also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some embodiments, the additional therapeutic agent is a chemotherapeutic agent (also referred to as an anti-neoplastic agent or anti-proliferative agent), e.g., an alkylating agent; an antibiotic; an anti-metabolite; a detoxifying agent; an interferon; a polyclonal or monoclonal antibody; an EGFR inhibitor; a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, an immune checkpoint inhibitor, a platinum based antineoplastic agent, a CDK inhibitor, a PARP inhibitor or any anti-neoplastic or anti-proliferative agent known to those of skill in the art.

Exemplary alkylating agents suitable for use according to the combination treatment modalities provided herein include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary suitable anthracyclines include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine-131 tositumomab (Bexxar); alemtuzumab (Campath); ibritumomab (Zevalin; In-111; Y-90 Zevalin); gemtuzumab (Mylotarg); eculizumab (Soliris) or denosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (EMD 72000) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/fasudil hydrochloride; flavopiridol; seliciclib (CYC202; Roscovitine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin), sorafenib (Nexavar), sunitinib (Sutent), ranibizumab, pegaptanib, or vandetinib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary immune checkpoint inhibitors include programmed cell death 1 (PD-1), and CD274 molecule (PD-L1) inhibitors. Exemplary PD-1 inhibitors include pembrolizumab, nivolumab and cemiplimab. Further examples of PD-1 inhibitors include retifanlimab, spartalizumab, camrelizumab, tislelizumab, toripalimab and dostarlimab. Exemplary PD-L1 inhibitors include atezolizumab, avelumab and durvalumab. Further examples of PD-L1 inhibitors include enfavolimab.

Exemplary platinum based antineoplastic agents include Cisplatin and Carboplatin.

Exemplary cyclin dependent kinase (CDK) inhibitors include abemaciclib, palbociclib, and ribociclib.

Exemplary poly (ADP-ribose) polymerase (PARP) inhibitors include talazoparib, olaparib, rucaparib, niraparib and veliparib.

Exemplary general chemotherapeutic, anti-neoplastic, anti-proliferative agents include, but are not limited to, altretamine (Hexalen); isotretinoin (Accutane; Amnesteem, Claravis, Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur—0.4 M 5-chloro-2,4-dihydroxypyrimidine-1 M potassium oxonate) and lovastatin.

Small molecule inhibitors refer to drugs that, because of their small, can be used to target both extracellular and intracellular proteins expressed by cancer cells. Small molecule inhibitors target serine/threonine/tyrosine kinases, matrix metalloproteinases (MMPs), heat shock proteins (HSPs), proteosome and other proteins playing a role in signal transduction pathways. Exemplary small molecule inhibitors include Acitinib, Erlotinib, Imatinib, Gefitinib, Sunitinib, Lapatinib, Nolitinib, Cabozantinib, Crizotinib, Sorafenib, Vemurafenib, Trametinib, Everolimus, Temisorolimus, Ruxolitinib, Bortezomib, Pazopanib, Ruzolitinib, Vandetenib, Bosutinib, Cabozantinib, Ponatinib, Regorafenib, Ibrutinib, Trametinib, Perifosine, Batimistat, Neovastat, Prinomastat, Rebimastat, Ganetespib, Marimastat, Obatoclax, Navitoclax and Carfilzomib.

In some embodiments, combination treatment modalities are provided in which the additional therapeutic agent is a cytokine, e.g., G-CSF (granulocyte colony stimulating factor).

In some embodiments, a pharmaceutical composition provided herein may be administered in combination with radiation therapy. Radiation therapy can also be administered in combination with a pharmaceutical composition provided herein and another chemotherapeutic agent described herein as part of a multi-agent therapy. In yet another aspect, a pharmaceutical composition provided herein may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), or CMFP (cyclophosphamide, methotrexate, 5-fluorouracil and prednisone).

In some preferred embodiments, a pharmaceutical composition provided herein may be administered with an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevec (targets Bcr-Ab1), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Ab1), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

In some embodiments, the antibody-drug conjugate or pharmaceutical composition comprising same is administered in combination with an adoptive cell therapy. In some embodiments, the adoptive cell therapy is a chimeric antigen receptor T cell (CAR T), or a CAR NK cell therapy.

### Dosing and Administration

In some embodiments, the methods comprise administering a biparatopic 5T4 antibody-drug conjugate. In some embodiments, the anti-5T4 antibody-drug conjugate or a pharmaceutical composition comprising same is administered to a subject daily, every 2 days, every 3 days, every 4 days, every 5 days, every 6 days, every 7 days, every 8 days, every 9 days, every 10 days, every 11 days, every 12 days, every 13 days, every 2 weeks, every 3 weeks, every 4 weeks, every 5 weeks, every 6 weeks, every 7 weeks, every 8 weeks, every 9 weeks, every 10 weeks, every 11 weeks, every 12 weeks, every 13 weeks, every 2 months, every 3 months or every 4 months.

In some embodiments, the anti-5T4 antibody-drug conjugate or a pharmaceutical composition comprising same is administered to a subject daily, every 2 days, every 3 days, every 4 days, every 5 days, every 6 days, every 7 days, every 8 days, every 9 days, every 10 days, every two weeks, every three weeks or monthly.

In some embodiments, the anti-5T4 antibody-drug conjugate or a pharmaceutical composition comprising same, is administered to a subject once a day. In some embodiments, the antibody-drug conjugate or a pharmaceutical composition comprising same is administered to a subject every two days. In some embodiments, the antibody-drug conjugate or composition comprising same is administered to a subject every three days. In some embodiments, the antibody-drug conjugate or composition comprising same is administered to a subject every four days. In some embodiments, the antibody-drug conjugate or composition comprising same is administered to a subject every five days. In some embodiments, the antibody-drug conjugate or composition comprising same is administered to a subject every six days. In some embodiments, the antibody-drug conjugate or composition comprising same is administered to a subject every seven days. In some embodiments, the antibody-drug conjugate or composition comprising same is administered to a subject every eight days. In some embodiments, the antibody-drug conjugate or composition comprising same is administered to a subject every nine days. In some embodiments, the antibody-drug conjugate or composition comprising same is administered to a subject every ten days. In some embodiments, the antibody-drug conjugate or composition comprising same is administered to a subject every eleven days. In some embodiments, the antibody-drug conjugate or composition comprising same is administered to a subject every twelve days. In some embodiments, the antibody-drug conjugate or composition comprising same is administered to a subject every thirteen days. In some embodiments, the antibody-drug conjugate or composition comprising same is administered to a subject every two weeks. In some embodiments, the antibody-drug conjugate or composition comprising same is administered to a subject every three weeks. In some embodiments, the antibody-drug conjugate or composition comprising same is administered to a subject every month. In some embodiments, the antibody-drug conjugate or pharmaceutical composition comprising same is administered to a subject two or more times a year. In some embodiments, the antibody-drug conjugate or pharmaceutical composition comprising same is administered to a subject two or more times every two years. In some embodiments, the antibody-drug conjugate or pharmaceutical composition comprising same is administered to a subject two or more times every two or more years.

In some embodiments, the antibody-drug conjugate or pharmaceutical composition comprising same is administered to a subject once every 7-14 days. In some embodiments, the antibody-drug conjugate or pharmaceutical composition comprising the same is administered to a subject once every 10-20 days. In some embodiments, the antibody-drug conjugate or pharmaceutical composition comprising the same is administered to a subject once every 5-15 days. In some embodiments, the antibody-drug conjugate or pharmaceutical composition comprising the same is administered to a subject once every 15-30 days.

In some embodiments, the antibody-drug conjugate or pharmaceutical composition comprising same is administered at least once every 36 hours. In some embodiments, the antibody-drug conjugate or pharmaceutical composition comprising same is administered at least once every 48 hours. In some embodiments, the antibody-drug conjugate or pharmaceutical composition comprising same is administered at least once every 60 hours. In some embodiments, the antibody-drug conjugate or pharmaceutical composition comprising same is administered at least once every 72 hours. In some embodiments, the antibody-drug conjugate or pharmaceutical composition comprising same is administered at least once every 84 hours. In some embodiments, the antibody-drug conjugate or pharmaceutical composition comprising same is administered at least once every 96 hours. In some embodiments, the antibody-drug conjugate or pharmaceutical composition comprising same is administered at least once every 5 days. In some embodiments, the antibody-drug conjugate or pharmaceutical composition comprising same is administered at least once every 6 days. In some embodiments, the antibody-drug conjugate or pharmaceutical composition comprising same is administered at least once every 7 days. In some embodiments, the antibody-drug conjugate or pharmaceutical composition comprising same is administered at least once every 8-10 days. In some embodiments, the antibody-drug conjugate or pharmaceutical composition comprising same is administered at least once every 10-12 days. In some embodiments, the antibody-drug conjugate or pharmaceutical composition comprising same is administered at least once every 12-15 days. In some embodiments, the antibody-drug conjugate or pharmaceutical composition comprising same is administered at least once every 15-25 days. In some embodiments, the antibody-drug conjugate or pharmaceutical composition comprising same is administered at least once every 20-30 days.

In some embodiments, the antibody-drug conjugate or pharmaceutical composition comprising same is administered to a subject at least once every 1 month, at least once every 2 months, at least once every 3 months, at least once every 4 months, or at least once every 6 months. In one embodiment, a dose of the antibody-drug conjugate or pharmaceutical composition comprising the same is administered at least once every 6-12 months. In some embodiments, the antibody-drug conjugate or pharmaceutical composition comprising same is administered quarterly. In some embodiments, the antibody-drug conjugate or pharmaceutical composition comprising same is administered daily, weekly, biweekly, monthly or annually. In some embodiments, the antibody-drug conjugate or pharmaceutical composition comprising same is administered once, twice, or two or more times a day, a week, a month or a year. In another embodiment, the dose is administered every two, three, four, or at least five years.

In some embodiments, administration of the antibody-drug conjugate or pharmaceutical composition comprising same comprises a dosing holiday. For example, the antibody-drug conjugate or pharmaceutical composition comprising same is administered to the subject every three days, followed by a week, two weeks, three weeks or a month with no administration, followed by a resumption of dosing. The person of ordinary skill in the art will understand that this dosing holiday is exemplary. Dosing holidays of other duration and frequency are contemplated as within the scope of the instant disclosure.

In some embodiments, the antibody-drug conjugate or pharmaceutical composition is administered for at least one week, at least two weeks, at least three weeks, at least 4 weeks, at least 5 weeks, at least 6 weeks, at least 2 months, at least 3 months, at least 4 months, at least 5 months, at least 6 months, at least 7 months, at least 8 months, at least 9 months, at least 10 months, at least 11 months, at least 12 months, at least 14 months, at least 16 months, at least 18 months, at least 20 months, at least 22 months, at least 2 years, at least 2.5 years or at least 3 years.

In some embodiments, the anti-5T4 antibody-drug conjugate pharmaceutical composition comprising same is administered at a dose of 0.01 μg/kg to 50.0 mg/kg, 0.01 μg/kg to 30 mg/kg, 0.01 μg/kg to 20 mg/kg, 0.01 μg/kg to 10 mg kg, 0.01 μg/kg to 1.0 mg/kg, 0.01 μg/kg to 100 μg/kg, 1 μg/kg to 50 mg/kg, 1 μg/kg to 30 mg/kg, 1 μg/kg to 20 mg/kg, 1 μg/kg to 10 mg kg, 1 μg/kg to 1.0 mg/kg, 1 μg/kg to 100 μg/kg, 10 μg/kg to 50.0 mg/kg, 10 μg/kg to 30 mg/kg, 10 μg/kg to 20 mg/kg, 10 μg/kg to 10 mg kg, 10 μg/kg to 1.0 mg/kg, 10 μg/kg to 100 μg/kg, 20 μg/kg to 50.0 mg/kg, 20 μg/kg to 30 mg/kg, 20 μg/kg to 20 mg/kg, 20 μg/kg to 10 mg kg, 20 μg/kg to 1.0 mg/kg, 20 μg/kg to 100 μg/kg, 50 μg/kg to 50.0 mg/kg, 50 μg/kg to 30 mg/kg, 50 μg/kg to 20 mg/kg, 50 μg/kg to 10 mg kg, 50 μg/kg to 5.0 mg/kg, 50 μg/kg to 4.0 mg/kg, 50 μg/kg to 3.0 mg/kg, 50 μg/kg to 2.0 mg/kg, 50 μg/kg to 1.0 mg/kg, 50 μg/kg to 100 μg/kg, 100 μg/kg to 50.0 mg/kg, 100 μg/kg to 30 mg/kg, 100 μg/kg to 20 mg/kg, 100 μg/kg to 10 mg kg, 100 μg/kg to 5.0 mg/kg, 100 μg/kg to 4.0 mg/kg, 100 μg/kg to 3.0 mg/kg, 100 μg/kg to 2.0 mg/kg, 100 μg/kg to 1.0 mg/kg, 1 mg/kg to 50.0 mg/kg, 1 mg/kg to 30 mg/kg, 1 mg/kg to 20 mg/kg, 1 mg/kg to 10 mg kg, 1 mg/kg to 5.0 mg/kg, 1 mg/kg to 4.0 mg/kg, 1 mg/kg to 3.0 mg/kg, 1 mg/kg to 2.0 mg/kg, 3 mg/kg to 50.0 mg/kg, 3 mg/kg to 30 mg/kg, 3 mg/kg to 20 mg/kg, 3 mg/kg to 10 mg kg, 3 mg/kg to 5.0 mg/kg, 5 mg/kg to 50.0 mg/kg, 5 mg/kg to 30 mg/kg, 5 mg/kg to 20 mg/kg, or 5 mg/kg to 10 mg kg.

In some embodiments, the anti-5T4 bispecific antibody-drug conjugate or pharmaceutical composition comprising same is administered at a dose of 0.01 μg/kg to 50 mg/kg. In some embodiments, the antibody-drug conjugate or pharmaceutical composition comprising same is administered at a dose of 20 μg/kg to 20 mg/kg. In some embodiments, the antibody-drug conjugate or pharmaceutical composition comprising same is administered at a dose of 1 mg/kg to 10.0 mg/kg. In some embodiments, the antibody-drug conjugate or pharmaceutical composition comprising same is administered at a dose of 0.1 μg/kg to 10.0 mg/kg.

In some embodiments, the antibody-drug conjugate or pharmaceutical composition comprising the same is administered to a subject in a dose ranging from 10 μg to 5000 mg, 10 μg to 4000 mg, 10 μg to 3000 mg, 10 μg to 2000 mg, 10 μg to 1000 mg, 10 μg to 750 mg, 10 μg to 500 mg, 10 μg to 400 mg, 10 μg to 300 mg, 10 μg to 200 mg, 10 μg to 100 mg, 100 μg to 50 mg, 100 μg to 40 mg, 100 μg to 30 mg, 100 μg to 20 mg, 100 μg to 10 mg, 100 μg to 5 mg, 100 μg to 4 mg, 100 μg to 3 mg, 300 μg to 5000 mg, 300 μg to 4000 mg, 300 μg to 3000 mg, 300 μg to 2000 mg, 300 μg to 1000 mg, 300 μg to 500 mg, 300 μg to 400 mg, 300 μg to 300 mg, 300 μg to 200 mg, 300 μg to 100 mg, 500 μg to 50 mg, 500 μg to 40 mg, 500 μg to 30 mg, 500 μg to 20 mg, 500 μg to 10 mg, 500 μg to 5 mg, 500 μg to 4 mg, 500 μg to 3 mg, 500 μg to 2 mg, 500 μg to 1 mg, 1 mg to 500 mg, 1 mg to 400 mg, 1 mg to 300 mg, 1 mg to 200 mg, 1 mg to 100 mg, 1 mg to 50 mg, 1 mg to 40 mg, 1 mg to 30 mg, 1 mg to 20 mg, 1 mg to 10 mg, 5 mg to 500 mg, 5 mg to 400 mg, 5 mg to 300 mg, 5 mg to 200 mg, 5 mg to 100 mg, 5 mg to 50 mg, 5 mg to 40 mg, 5 mg to 30 mg, 5 mg to 20 mg, 5 mg to 10 mg, 10 mg to 500 mg, 10 mg to 400 mg, 10 mg to 300 mg, 10 mg to 200 mg, 10 mg to 100 mg, 10 mg to 50 mg, 20 mg to 500 mg, 20 mg to 300 mg, 20 mg to 200 mg, 5 mg to 100 mg, or 20 mg to 50 mg.

In some embodiments, the antibody-drug conjugate or pharmaceutical composition comprising the same is administered to a subject in a dose ranging from 0.05 μg to 5,000 mg. In some embodiments, the antibody-drug conjugate or pharmaceutical composition comprising the same is administered to a subject in a dose ranging from 1 mg to 3,000 mg. In some embodiments, the antibody-drug conjugate or pharmaceutical composition comprising the same is administered to a subject in a dose ranging from 10 mg to 2,000 mg. In some embodiments, the antibody-drug conjugate or pharmaceutical composition comprising the same is administered to a subject in a dose ranging from 1.0 μg to 1,000 mg. In some embodiments, the antibody-drug conjugate or pharmaceutical composition comprising the same is administered to a subject in a dose ranging from 50 μg to 500 mg. In some embodiments, the antibody-drug conjugate or pharmaceutical composition comprising the same is administered to a subject in a dose ranging from 100 μg to 500 mg. In some embodiments, the antibody-drug conjugate or pharmaceutical composition comprising the same is administered to a subject in a dose ranging from 100 μg to 100 mg. In some embodiments, the antibody-drug conjugate or pharmaceutical composition comprising the same is administered to a subject in a dose ranging from 500 μg to 1000 mg. In some embodiments, the antibody-drug conjugate or pharmaceutical composition comprising the same is administered to a subject in a dose ranging from 500 μg to 100 mg. In some embodiments, the antibody-drug conjugate or pharmaceutical composition comprising the same is administered to a subject in a dose ranging from 1 mg to 500 mg. In some embodiments, the antibody-drug conjugate or pharmaceutical composition comprising the same is administered to a subject in a dose ranging from 1 mg to 100 mg. In some embodiments, the antibody-drug conjugate or pharmaceutical composition comprising the same is administered to a subject in a dose ranging from 500 μg to 50 mg. In some embodiments, the antibody-drug conjugate or pharmaceutical composition comprising the same is administered to a subject in a dose ranging from 5 mg to 1,000 mg. In some embodiments, the antibody-drug conjugate or pharmaceutical composition comprising the same is administered to a subject in a dose ranging from 100 mg to 1,000 mg In some embodiments, the antibody-drug conjugate or pharmaceutical composition comprising the same is administered to a subject in a dose ranging from 10 mg to 500 mg. In some embodiments, the antibody-drug conjugate or pharmaceutical composition comprising the same is administered to a subject in a dose ranging from 10 mg to 100 mg.

In one embodiment, a single one time dose of the antibody-drug conjugate or pharmaceutical composition comprising the same is administered to a subject. In another embodiment, a total of two doses are administered to the subject. In another embodiment, a total of two or more doses are administered to the subject. In some embodiments, a single dose is administered in a single injection. In some embodiments, a single dose is administered in multiple injections, e.g. 1, 2, 3, 4, or more injections.

In some embodiments, the antibody-drug conjugate or pharmaceutical composition comprising same is administered by intravenous, intra-arterial, subcutaneous, intratumoral or intramuscular injection of a liquid preparation. In some embodiments, liquid formulations include solutions, suspensions, dispersions, emulsions, oils and the like. In some embodiments, the antibody-drug conjugate or pharmaceutical compositions are administered intravenously, and are thus formulated in a form suitable for intravenous administration. In some embodiments, the antibody-drug conjugate or pharmaceutical compositions are administered intra-arterially, and are thus formulated in a form suitable for intra-arterial administration. In some embodiments, the antibody-drug conjugate or pharmaceutical compositions are administered subcutaneously, and are thus formulated in a form suitable for subcutaneous administration. In some embodiments, the antibody-drug conjugate or pharmaceutical compositions are administered intratumorally, and are thus formulated in a form suitable for intratumoral administration.

In some embodiments, compositions for use in the methods disclosed herein comprise solutions or emulsions, which in some embodiments are aqueous solutions or emulsions comprising a safe and effective amount of the compounds disclosed herein and optionally, other compounds, intended for intravenous or subcutaneous administration.

In some embodiments, administration of the antibody-drug conjugate described herein, or pharmaceutical compositions comprising same increases 5T4 receptor cellular internalization of cancer cells in a subject. Alternatively, or in addition, administration of the antibody-drug conjugate described herein, or pharmaceutical compositions comprising same decreases the viability of cancer cells in a subject. Alternatively, or in addition, administration of the antibody-drug conjugate described herein, or pharmaceutical compositions comprising same decreases the viability of cancer cells. Alternatively, or in addition, administration of the antibody-drug conjugate described herein, or pharmaceutical compositions comprising same reduces tumor growth in a subject. Alternatively, or in addition, administration of the antibody-drug conjugate described herein, or pharmaceutical compositions comprising same increases the survival of a subject. Alternatively, or in addition, administration of the antibody-drug conjugate described herein, or pharmaceutical compositions comprising same reduces a sign or symptom of cancer. Alternatively, or in addition, administration of the antibody-drug conjugate described herein, or pharmaceutical compositions comprising same reduces tumor volume, tumor number, slows rate of tumor growth, or a combination thereof.

### Kits and Articles of Manufacture

The disclosure further provides kits comprising the anti-5T4 antigen binding domains, anti-5T4 antibodies, and antibody-drug conjugates of the disclosure or pharmaceutical composition comprising same for preventing, treating or delaying cancer in a subject, wherein the kit comprises one or more doses of pharmaceutical composition and instructions on how to use the pharmaceutical preparation or composition.

The disclosure further provides kits comprising polynucleotides or vectors encoding the 5T4 antibodies of the disclosure.

The disclosure further provides kits comprising cells comprising the 5T4 targeting receptors of the disclosure.

In some embodiments, the kit comprises syringes, vials labels, and/or instructions on how to use the pharmaceutical preparation or composition.

In some embodiments of the kits of the disclosure, the various constituents of the compositions come pre-measured and/or prepackaged and/or ready for use without additional measurement, etc. The present invention also optionally comprises kits for conducting/using the methods and/or the compositions of the invention. In particular, these kits optionally include, e.g., appropriate antibody-drug conjugate (and optionally additional reagents for performing combination treatments as described supra). Additionally, such kits can also comprise appropriate excipients (e.g., pharmaceutically acceptable excipients) for performing therapeutic and/or prophylactic treatments of the invention. Such kits optionally contain additional components for the assembly and/or use of the compositions of the invention including, but not limited to, e.g., diluents, etc.

The compositions described herein are optionally packaged to include all (or almost all) necessary components for performing the methods of the invention or for using the compositions of the invention (optionally including, e.g., written instructions for the use of the methods/compositions of the invention). For example, the kits can optionally include such components as, e.g., buffers, reagents, serum proteins, antibodies, substrates, etc. In the case of prepackaged reagents, the kits optionally include pre-measured or pre-dosed amounts that are ready to incorporate into the methods without measurement, e.g., pre-measured fluid aliquots, or pre-weighed or pre-measured solid reagents that can be easily reconstituted by the end-user of the kit.

Such kits also typically include appropriate instructions for performing the methods of the invention and/or using the compositions of the invention. In some embodiments, the components of the kits/packages are provided in a stabilized form, so as to prevent degradation or other loss during prolonged storage, e.g., from leakage. A number of stabilizing processes/agents are widely used for reagents, etc. that are to be stored, such as the inclusion of chemical stabilizers (i.e., enzymatic inhibitors, microbicides/bacteriostats, anticoagulants), and the like.

The following examples are presented in order to more fully illustrate the preferred embodiments of the invention. They should in no way be construed, however, as limiting the broad scope of the invention.

### EXAMPLES

### Example 1: Methods for Examples 2-3

### Cell Culture

A human embryonic kidney 293 (HEK293) cell line transfected with 5T4 and the human prostate cancer cell line DU145 were maintained in EMEM medium supplemented with 10% heat-inactivated fetal bovine serum (FBS). The human breast cancer cell line T-47D was maintained in RPMI-1640 medium supplemented with 10% heat-inactivated FBS and 0.01 mg/mL human recombinant insulin (Gibco). The human breast cancer cell line MCF-7 was maintained in EMEM medium supplemented with 10% heat-inactivated FBS and 0.01 mg/mL human recombinant insulin (Gibco). All cancer cell lines were cultured in the presence of 1% antibiotics penicillin-streptomycin and in a humidified atmosphere of 5% CO2 and at a temperature of 37° C.

### Epitope Binding

To examine epitope binding of anti-5T4 bispecific antibody-drug conjugate parental monospecific antibodies, surface plasmon resonance (SPR) binding experiments were performed using Biacore™ 8K systems (Cytiva™). HBS-EP+ buffer (10 mM HEPES, 150 mM NaCl, 3 mM EDTA and 0.05% v/v Surfactant P20) was used as running buffer. Anti-mouse IgG antibodies from the mouse antibody capture kit (Cytiva) were coupled in active flow cells on a CM5 sensor chip using an amine coupling kit (Cytiva), whereas the reference cell was left blank. As the first step, mouse monospecific antibody Ab1 or Ab2 was injected over both cells on all experimental channels. Second, test article 5T4 or buffer was injected over both cells on the corresponding channel. Third, humanized monospecific antibody Ab1 or Ab2, or buffer, was injected over both cells on all experimental channels. After each binding cycle, the sensor chip was regenerated with 10 mM Glycine-HCl pH1.7. SPR signals (RU) were recorded in real-time and plotted against time to generate binding sensorgrams. Sensorgrams from multiple channels were overlaid using Biacore 8K Evaluation Software.

### Target Binding Assay

The binding affinity of anti-5T4 bispecific antibody-drug conjugate monospecific and bispecific antibodies to 5T4 was measured by Biacore kinetic experiments using a Protein A sensor chip on Biacore 8K (Cytiva) with HBS-EP+ as running buffer. Anti-5T4 bispecific antibody-drug conjugate antibodies were captured to a target level of around 100 resonance units (RU) on the active cell of a protein A sensor chip. 5T4 protein was serially diluted to 10, 5, 2.5, 1.25, and 0.625 nM with HBS-EP+ buffer and injected over surfaces of both active and reference cells at a flow rate of 30 NL/min. After each binding cycle, the sensor chip was regenerated with glycine solution, pH 1.5. Signals from reference surface and an ensemble of buffer blank injections were subtracted from binding signals to correct for nonspecific binding and injection artifacts. The corrected experimental signals were globally fitted to a 1:1 binding model to extract kinetic constants, including association rate constant (ka), dissociation rate constant (kd), and equilibrium dissociation constant (KD), using the Biacore 8K Evaluation Software.

### FcγR Binding Activity

Site mutations (L234F and S239C) in the Fc region of anti-5T4 bispecific antibody-drug conjugates were designed to mitigate potential side effects which could be caused by the antibody effector function. To confirm the mutations had the intended effect of reducing Fc receptor binding activities, the affinities between an anti-5T4 bispecific antibody with Fc mutations and an array of Fc receptors were evaluated by Biacore assay. A total of 5 Fc receptors, including FcγRI (Abcam), FcγRIIa, FcγRIIb, FcγRIIIa (158V), and FcγRIIIb (Sino Biological), were analyzed. One parental anti-5T4 Antibody (Ab1) without Fc mutations, was used as a positive control for FcR binding. SPR kinetic experiments were performed on Biacore 8K systems (GE Healthcare) with HBS-EP+ as running buffer. Antibodies were captured in the active flow cell of a protein A sensor chip (Cytiva). Capture level for each ligand was maintained between 50-100RU. For kinetic analysis, each receptor was serially diluted to 6 different concentrations, ranging from 0.41 nM to 100 nM, and the serial dilutions were injected sequentially over both reference and active flow cells in each single channel. After each binding cycle, the sensor chip was regenerated with glycine solution, pH 1.5. The resulting sensorgrams were fitted with a one-to-one binding model to extract kinetic constants using Biacore 8K Evaluation Software.

### Internalization Assay

5T4-expressing cancer cells (DU145, PANC-1, T-47D, MCF7 and 5T4-transfected HEK293 cells—clones 3G9 and 4F2—were incubated with antibody (10 μg/ml each antibody) on ice for 1 hour and then washed to remove unbound antibodies. An aliquot of cells remained on ice and the rest were incubated at 37° C. for different periods of time, then stained with FITC-labeled rabbit antibody against human IgG Fc (Invitrogen®). Cells were fixed in 2% paraformaldehyde overnight and analyzed by flow cytometry and FlowJo® software. Receptor-antibody complex internalization at each time point was calculated as percent mean fluorescent intensity (MFI) loss at 37° C. relative to that on ice after subtracting the background value of MFI derived from the untreated control.

### In Vitro Cytotoxicity Assay

The human cancer cell lines AGS, DU145, T-47D, MCF-7 and 5T4-transfected HEK293 (HEK293-5T4) cells were seeded in 96-well flat bottom plates at a density of 1,000 to 10,000 cells per 80 μL per well, depending on the growth kinetics of each cell line. An anti-5T4 bispecific antibody-drug conjugate or control articles were prepared at 5× working concentration in a stepwise 1:4 serial dilution series using the culture medium, and then added to the cells at 20 μL/well in triplicates. Cells were incubated at 37° C./5% CO₂ for 72 days. Cell viability was determined using Cell Titer-Glo 2.0 Luminescent Viability Assay (Promega®) according to the manufacturer's instructions. Data were analyzed using the GraphPad Prism® software and presented as percent viability or percent growth inhibition relative to the untreated control. Growth inhibition was calculated using the formula: 1-(RLUT-RLU0)/(RLUU-RLU0). RLU: relative light unit; RLU0: RLU of initial cell seeding before treatment; RLUT: RLU of treated group at the end of the experiment. RLUU: RLU of untreated control at the end of the experiment.

### Enzyme-Linked Immunosorbent Assays (ELISAs)

Briefly, 2 μg/mL of recombinant 5T4 from human, non-human primate (cynomolgus/rhesus macaque), mouse and rat (Sino biologics and Biorbyt) was coated on high protein binding 96 well ELISA plates. Plates were blocked with 2% bovine serum albumin (BSA) in phosphate-buffered saline (PBS) overnight. A dilution curve of bispecific antibody ranging from 25 μg/ml-12.5 ng/mL was plated. Bound bispecific antibody was detected with an anti-human IgG—HRP (1:10,000) and TMB (BD biosciences). Developed TMB signals were read at 450 nM on an iD5 plate reader (Molecular Devices).

### Detection of 5T4 by Cells In Vitro

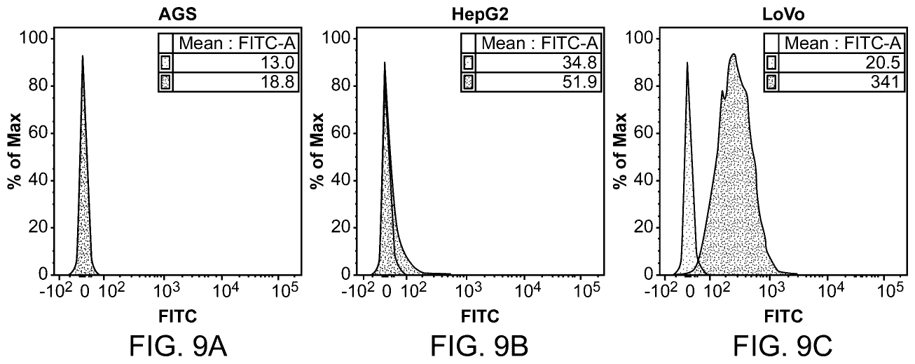

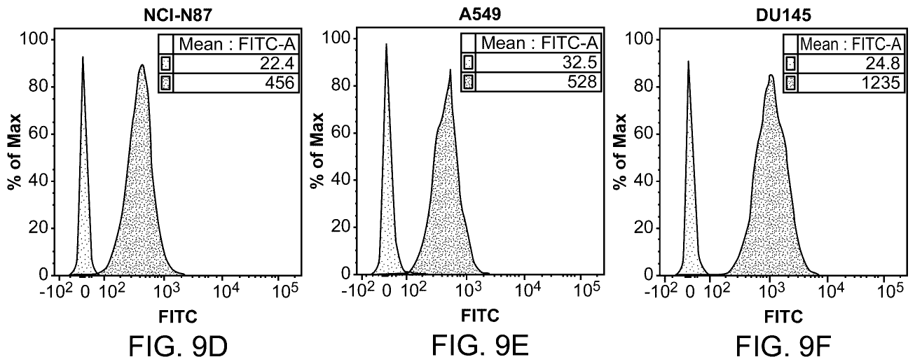

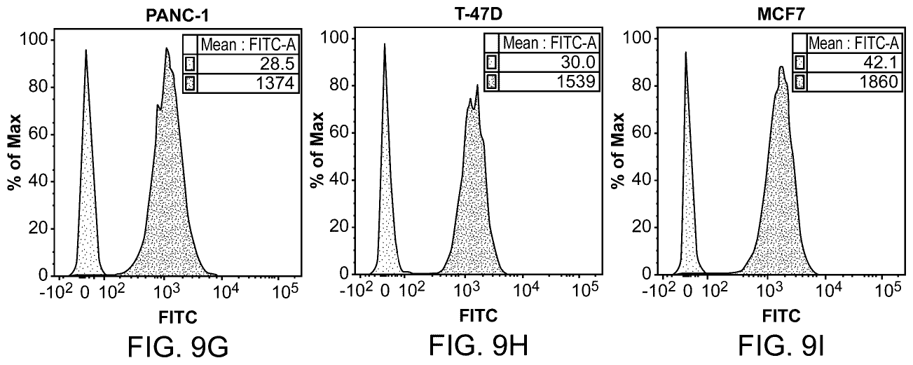

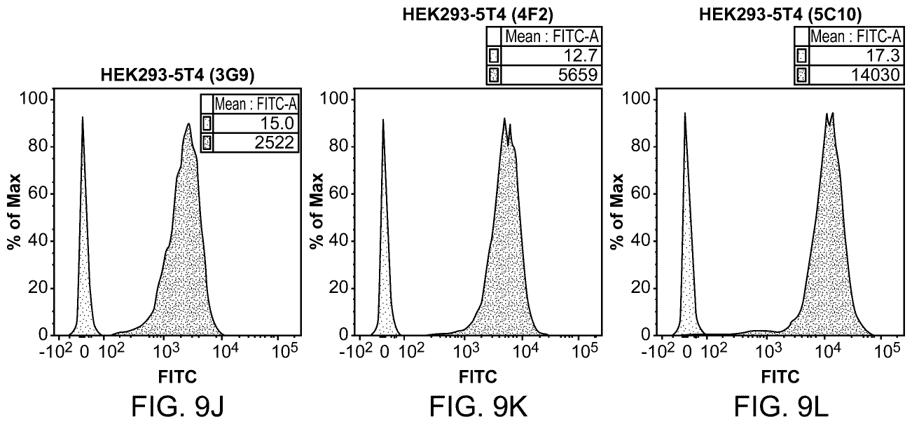

Briefly, select cells were incubated with or without anti-5T4 humanized antibody Ab1 followed by detection with FITC-conjugated anti-human IgG antibody. Target-specific binding of cell surface 5T4 by anti-5T4 primary antibody (h)Ab1 was detected by FITC-conjugated anti-human IgG secondary antibody and mean fluorescent intensity (MFI) determined by flow cytometry is shown in FIGS. 9A-9L. Staining with secondary antibody only (in the absence of primary antibody) serves as a negative control for background signal.

### In Vivo Tumor Growth Model

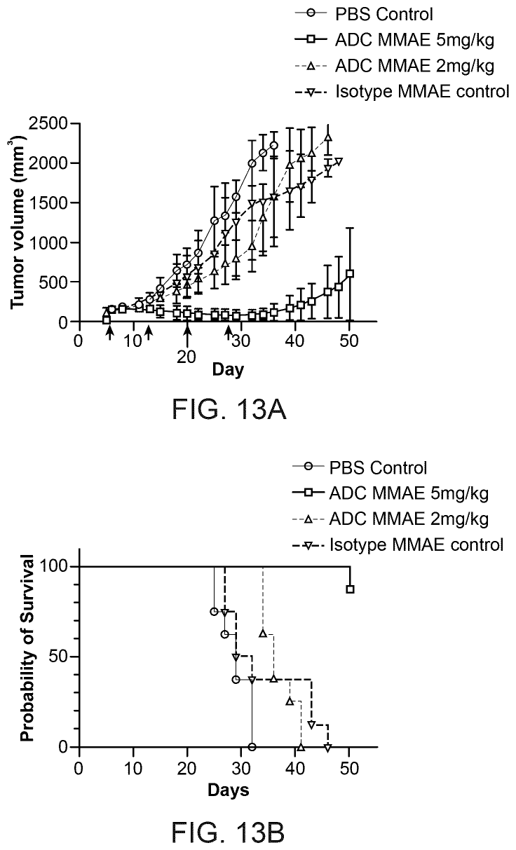

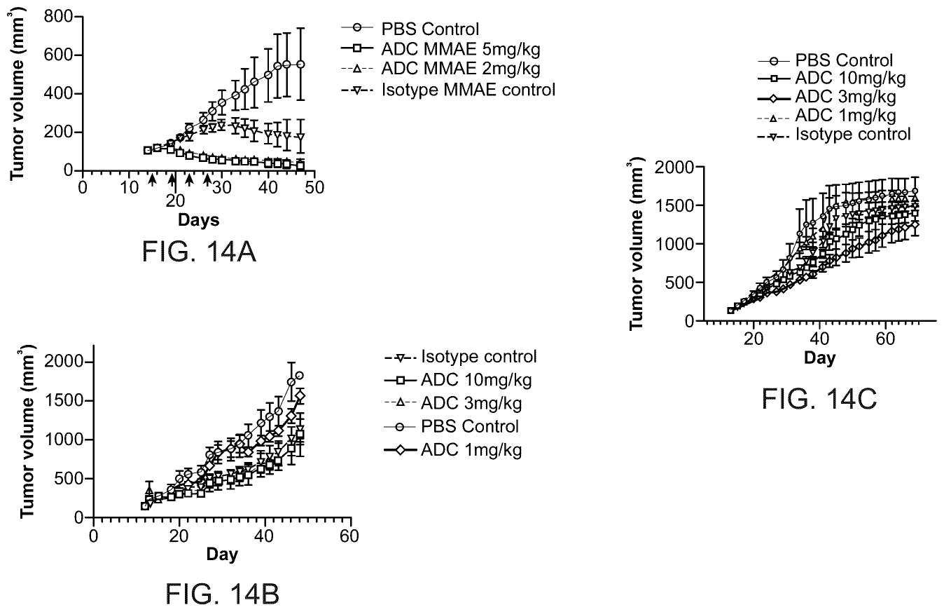

Mice were implanted with human CDX tumors and then treated with 5T4 biparatopic antibodies, isotype control antibody coupled with monomethyl auristatin E (MMAE) or mock PBS treatment. Female nu/nu mice implanted with 60-day estrogen pellets (FIG. 13C only) were implanted with 3 million CDX cancer cells that had been mixed 50/50 with Matrigel. Mice bearing tumors that were at least 150 mm and had three successive increases in tumor volume were randomized into treatment groups. Animals were treated with the noted dosage of 5T4 biparatopic antibody or relevant control QD4×4 (FIGS. 13A and 14A) or QD7×2 (FIGS. 14B and 14C). Tumors were measured by caliper every 3 days. A survival curve was plotted for the aggressive H1975 tumor model (FIG. 13B).

### Example 2: Improved Cytotoxicity and Internalization Mediated by 5T4 Bispecific Antibodies

Non-overlapping epitope binding assay (SPR)

To confirm the non-overlapping epitope binding of the select monospecific antibody Nos. 1 and 2, a surface plasmon resonance (SPR) technology-based binding assay was performed. Briefly, mouse antibody No. 1 ((m)Ab1) or mouse antibody No. 2 ((m)Ab2) were captured on the sensor chip immobilized with anti-mouse IgG antibodies (“mouse antibody loading”), followed by the injection of 5T4 (“mouse antibody binding”) and then humanized antibody No. 1 ((h)Ab1) or humanized antibody No. 2 ((h)Ab2) (“5T4 binding”). Detailed methods are provided in Example 1, above. Humanized antibodies (not captured by anti-mouse IgG antibodies) bind to 5T4 only when the corresponding epitopes on 5T4 are not occupied by mouse antibodies (captured by anti-mouse IgG antibodies).

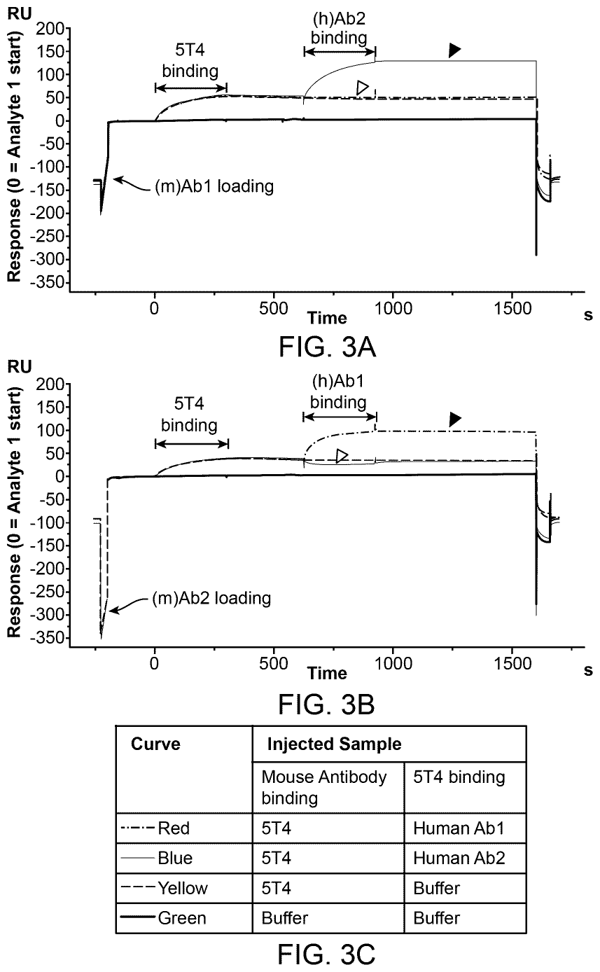

As shown in FIGS. 3A-3C, the attachment of mouse antibodies on the sensor chip could be visualized through the increase of signal on all 4 curves in the mouse antibody loading step. Loading of epitope-unmatched humanized antibody generated significant responses at both the mouse antibody binding and 5T4 binding steps (blue curve in FIG. 3A and red curve in FIG. 3B, filled arrowheads), confirming the non-overlapping epitope binding of the two monospecific antibodies Ab1 and Ab2. In contrast, loading of epitope-matched humanized antibody generated significant responses only at the mouse antibody binding step, but not at the 5T4 binding step (red curve in FIG. 3A and blue curve in FIG. 3B, unfilled arrowheads), indicating binding competition between epitope-matched mouse and humanized antibodies.

These data demonstrate that select monospecific antibodies Ab1 and Ab2 recognize non-overlapping epitopes on 5T4, and are thus suitable to be used for constructing the 5T4 biparatopic antibodies of the present disclosure.

### Binding Affinity of Bispecific Antibodies to 5T4

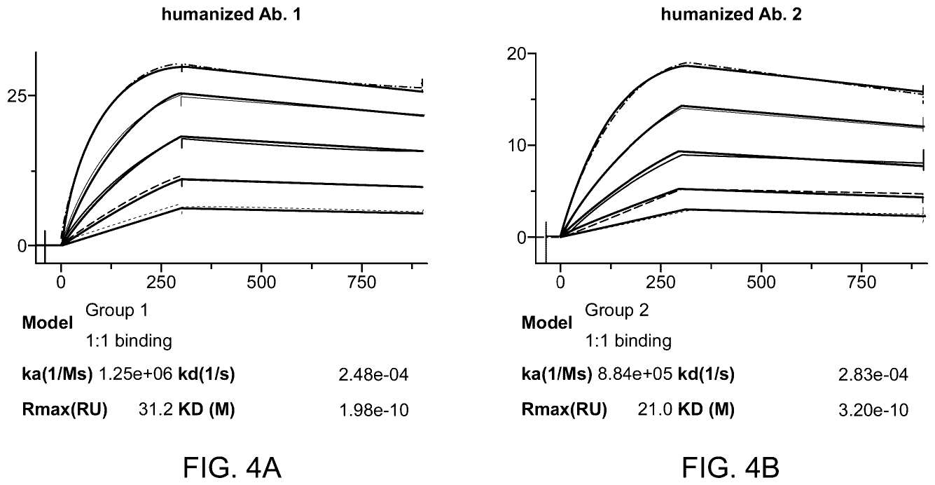

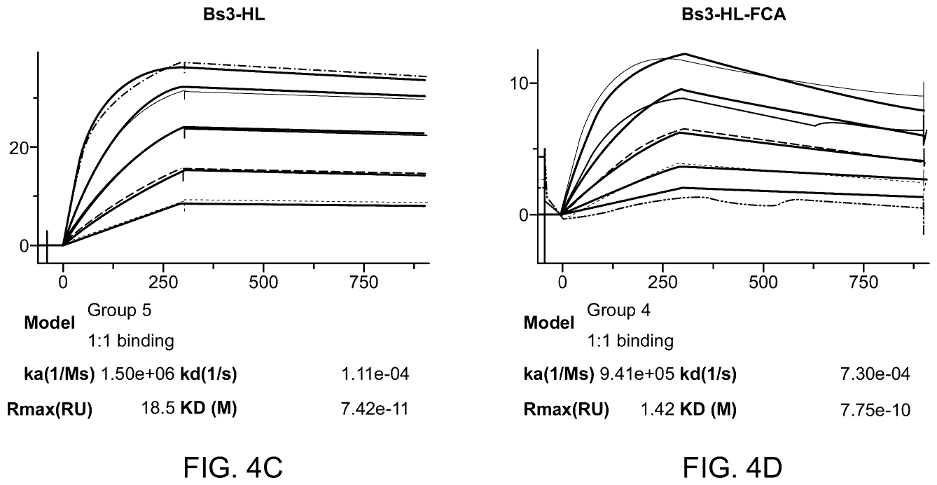

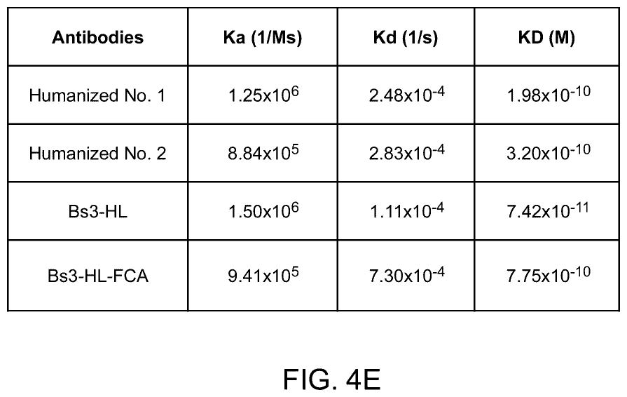

As shown in FIGS. 4A-4E, the equilibrium dissociation constant (KD) for humanized monospecific antibodies (h)Ab1 and (h)Ab2 (FIGS. 4A-4B) and bispecific antibodies (BsAbs) Bs3-HL (SEQ ID NO: 100, SEQ ID NO: 101) and Bs3-HL-FCA (FIGS. 4C-4D) were 1.98×10⁻¹⁰ M, 3.2×10⁻¹⁰ M, 7.42×10⁻¹⁰ M and 7.75×10⁻¹⁰ M, respectively (summarized in FIG. 4E). This data was generated using the methods described in Example 1. These data indicate that the anti-5T4 bispecific antibodies have overall similar binding affinity to 5T4 compared with the parental monospecific antibodies. Therefore, the construction of bispecific antibodies (BsAbs) and introduction of site mutations (L234F, S239C and N434A, abbreviated as FCA) in the Fc region do not significantly alter binding characteristics of the anti-5T4 bispecific antibody-drug conjugate.

Binding Affinity of Bispecific Antibodies to 5T4 from Multiple Species

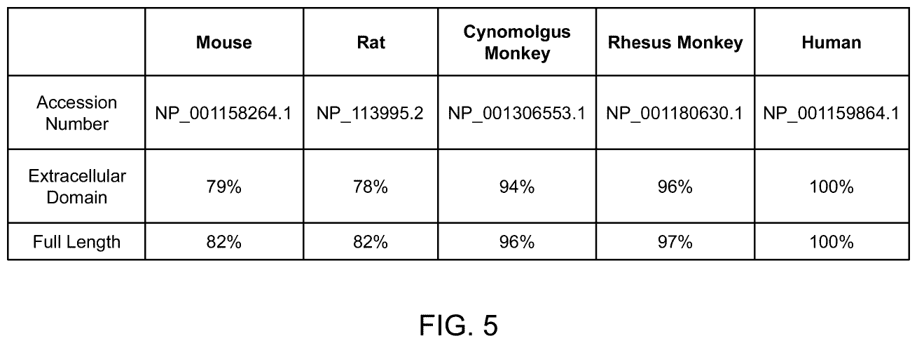

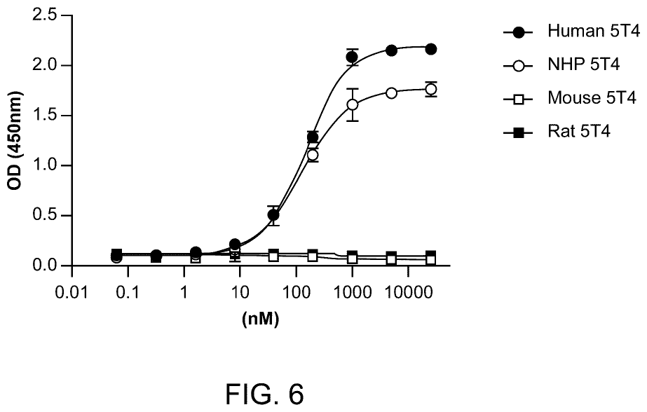

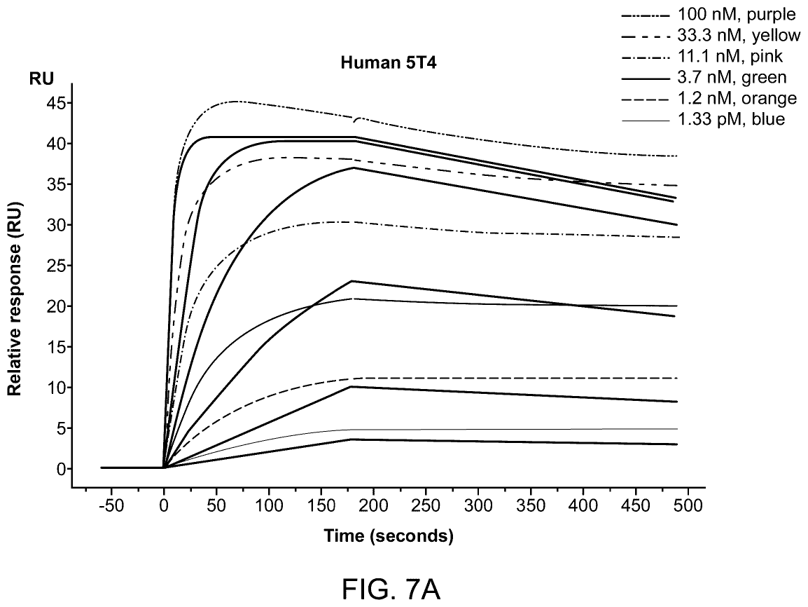

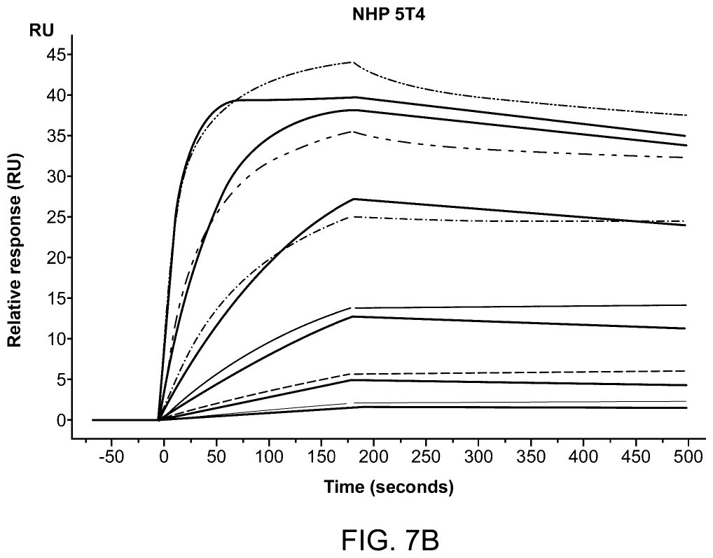

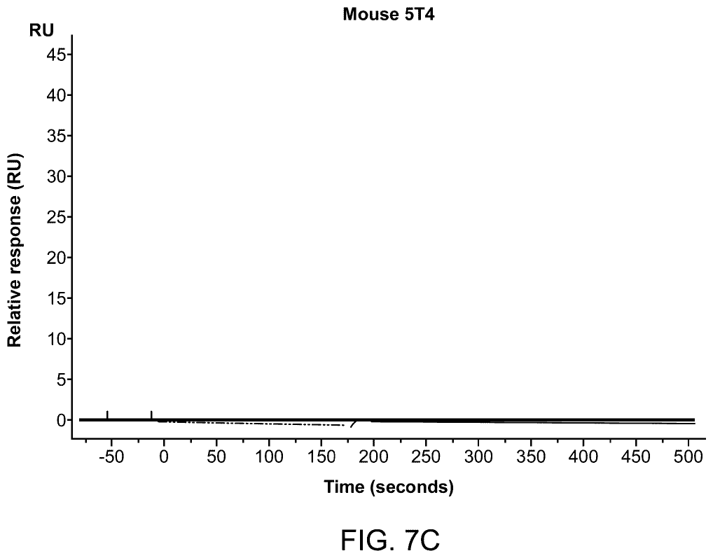

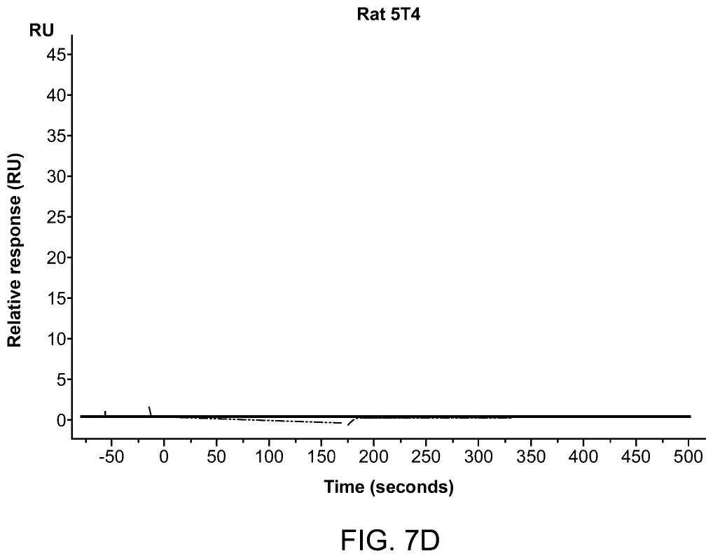

To determine the relevant animal model species for evaluation of exemplary bispecific antibodies, 5T4 from various species (percent identity to human 5T4 shown in FIG. 5) was evaluated for the ability of bispecific antibodies to bind to the 5T4 protein from that species in an ELISA assay, as shown in FIG. 6. Exemplary bispecific antibodies bound to human and non-human primate 5T4 proteins. However, no binding to either mouse or rat 5T4 was detected. Surface plasmon resonance binding of the bispecific antibody to 5T4 from various species are shown in FIGS. 7A-7D.

### Impact of Fc Site Mutations on Binding of Exemplary Bispecific Antibodies to Fc Gamma Receptors

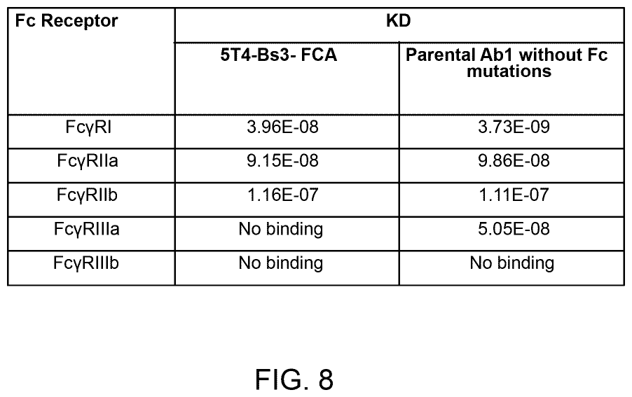

Site mutations (L234F, S239C and N434A) in the Fc region of the Bs3-HL biparatopic antibody are designed to mitigate potential side effects caused by the antibody effector function. To confirm that the mutations have reduced Fc receptor binding activities, the affinity between BsAb with the FCA mutations (Bs3-HL-FCA) and select Fc gamma receptors (FcγRs) were evaluated by Biacore® assay. As summarized in FIG. 8, kinetically derived KD values of BsAb binding to FcγRI showed 10-fold reduction in affinity compared with the BsAb without Fc mutations. The Fc mutations resulted in loss of binding to FcγRIIIa, thus preventing potentially unwanted interactions and immune mediated side effects.

To identify 5T4-negative and 5T4-positive cancer cell lines for in vitro and in vivo experiments, flow cytometry was used to determine the cell surface 5T4 expression level.

As shown in FIGS. 9A-9L, a gastric cancer cell line (AGS) and a liver cancer cell line (HepG2) were found to be negative (or almost negative) for 5T4, and three different stable clones from 5T4-transfected HEK293 cells (HEK293-5T4, clones 3G9, 4F2 and 5C10) showed different levels of 5T4 expression. In addition, the 5T4-positive cancer cells showed varying 5T4 expression levels that were generally lower than those of the 5T4-transfected cell lines. These include colorectal cancer (LoVo), gastric cancer (NCI-N87), lung cancer (A549), prostate cancer (DU145), pancreatic cancer (PANC-1) and two breast cancer (T-47D and MCF7) cell lines.

### Bispecific Antibodies Enhance 5T4 Internalization

To examine whether the exemplary bispecific antibodies can induce enhanced 5T4 internalization, 5T4-expressing cancer cell lines DU145, PANC-1, T-47D, MCF7 and 5T4-transfected HEK293 cells (clones 3G9 and 4F2) were treated with BsAbs as well as parental monospecific antibodies, and then the cell surface level of 5T4 was measured by flow cytometry. A flow cytometry-based in vitro internalization assay was used to determine the relative internalization rates of different antibodies into cancer cells that express different levels of 5T4.

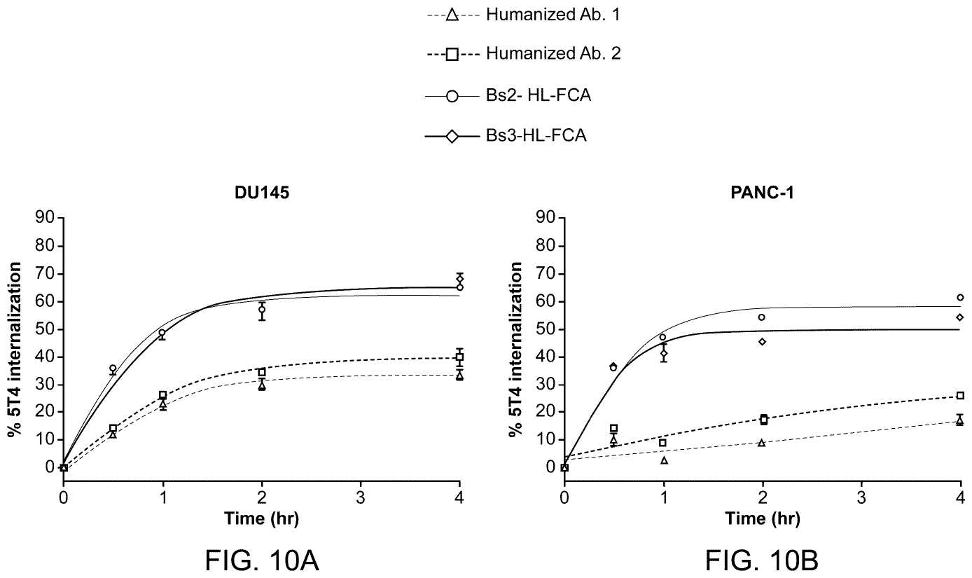

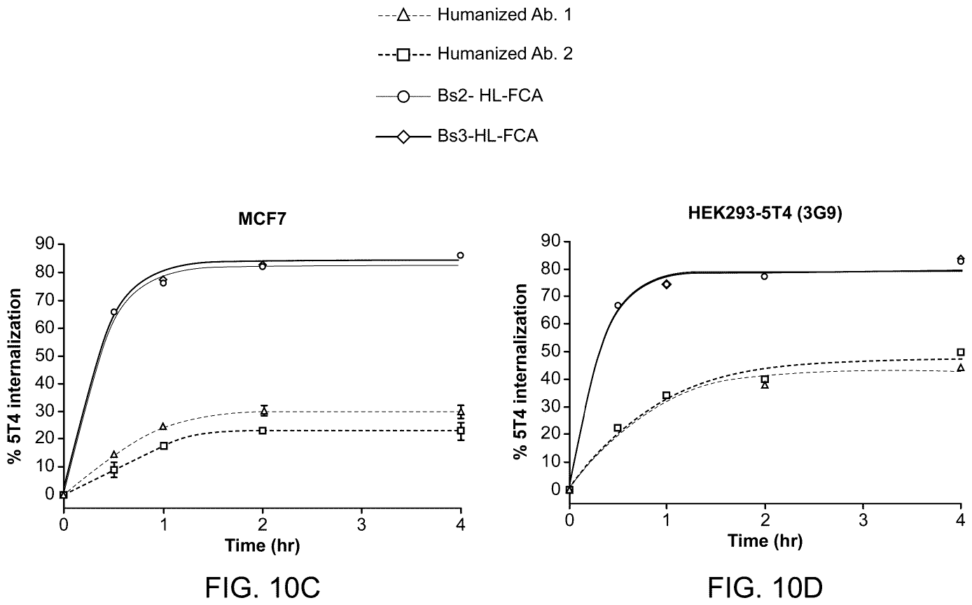

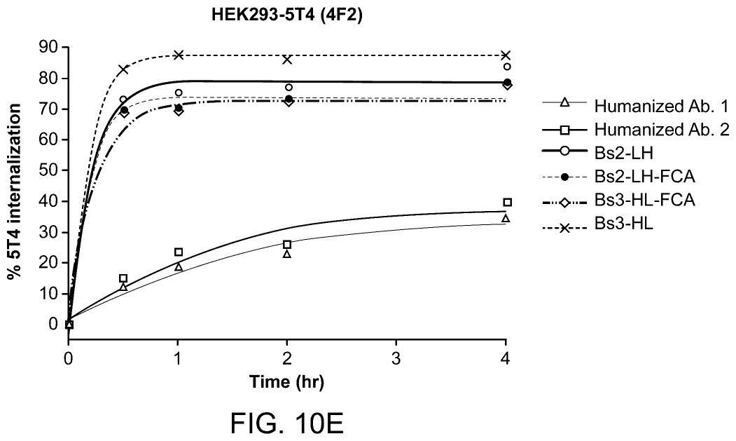

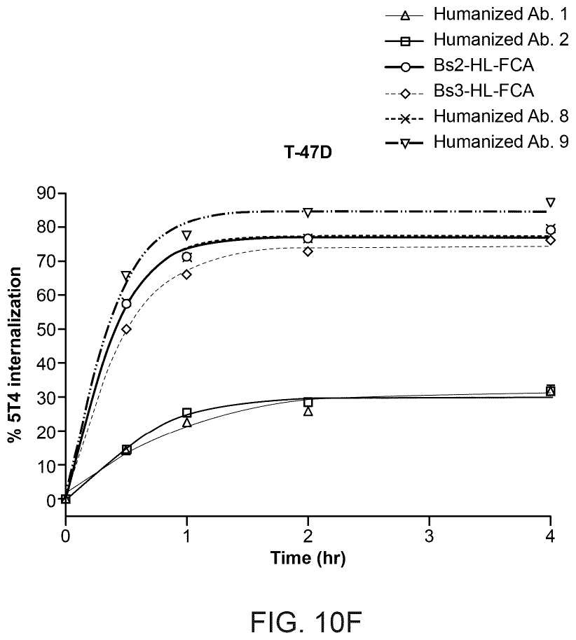

As shown in FIGS. 10A-10F, BsAbs elicited a much faster and higher level of receptor internalization than either monospecific antibody, and the extent of increase in internalization induced by BsAbs was largely influenced by the 5T4 level on the cell surface. Representative graphs show the mean percent internalization ±SEM (n=3) in prostate (DU145) (FIG. 10A), pancreatic (PANC-1) (FIG. 10B), two breast cancer (T-47D and MCF7) cell lines (FIG. 10F and FIG. 10C, respectively) and 5T4-transfected HEK293 cells (clones 3G9 and 4F2) (FIG. 10D and FIG. 10E, respectively).

### Cytotoxicity Activity of Exemplary Bispecific 5T4 Antibodies

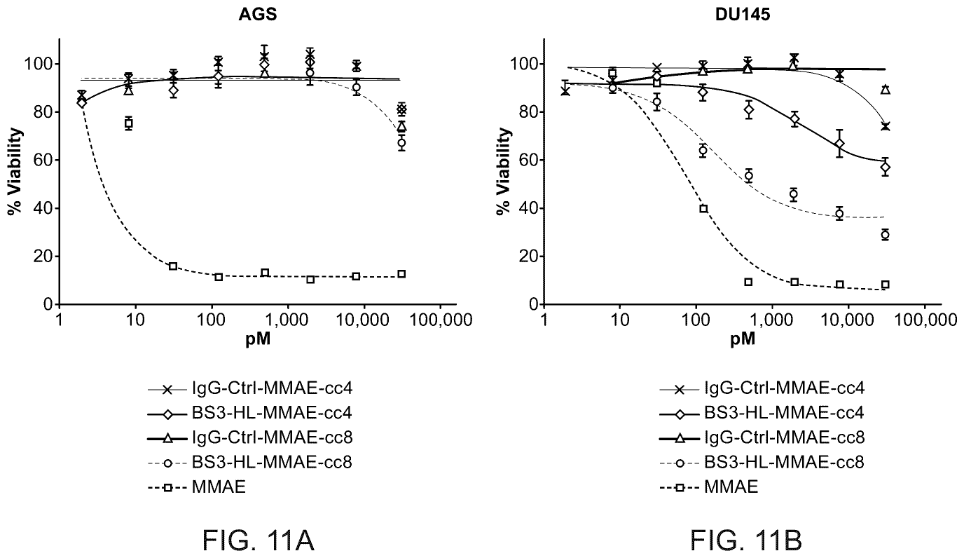

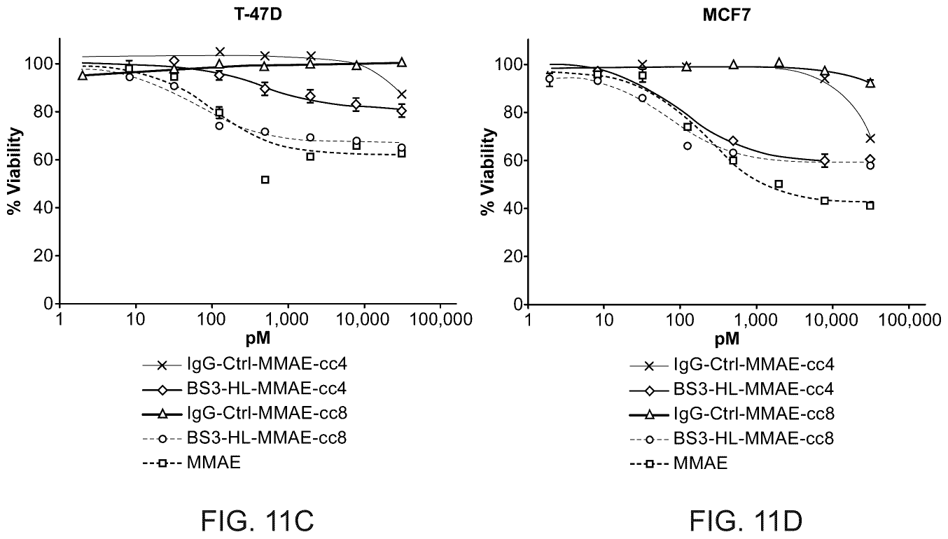

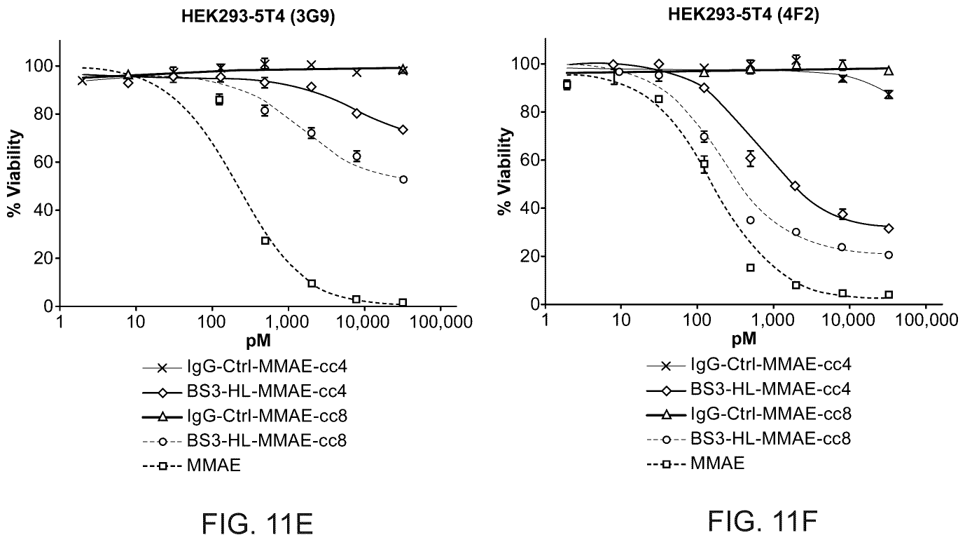

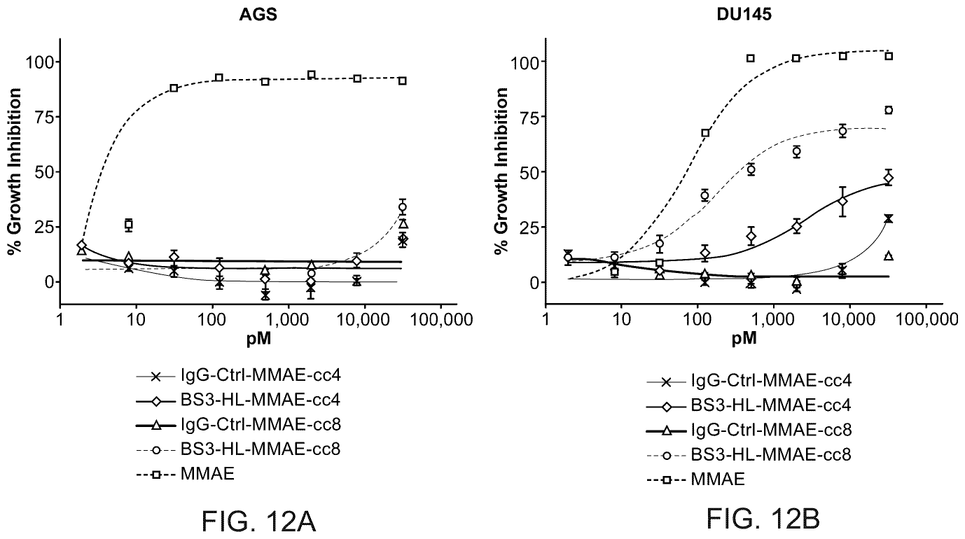

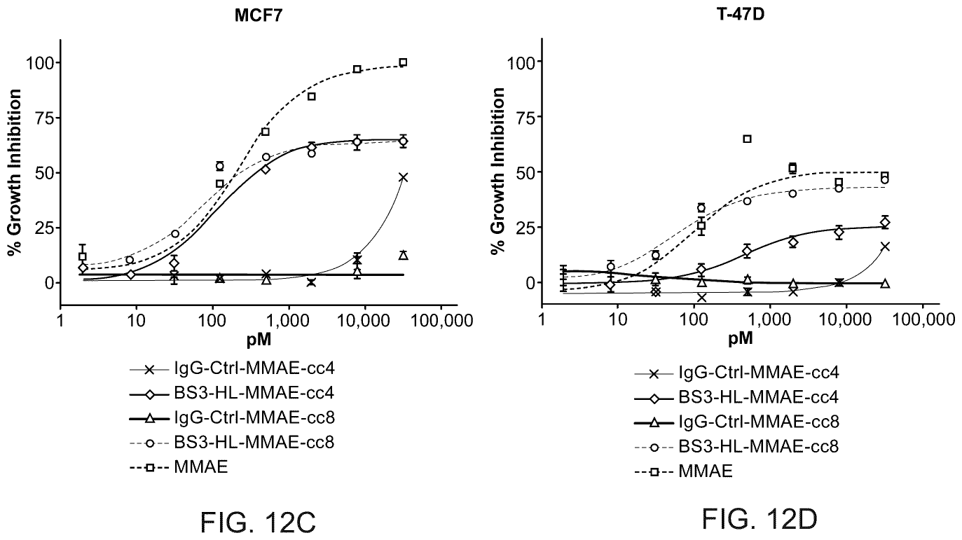

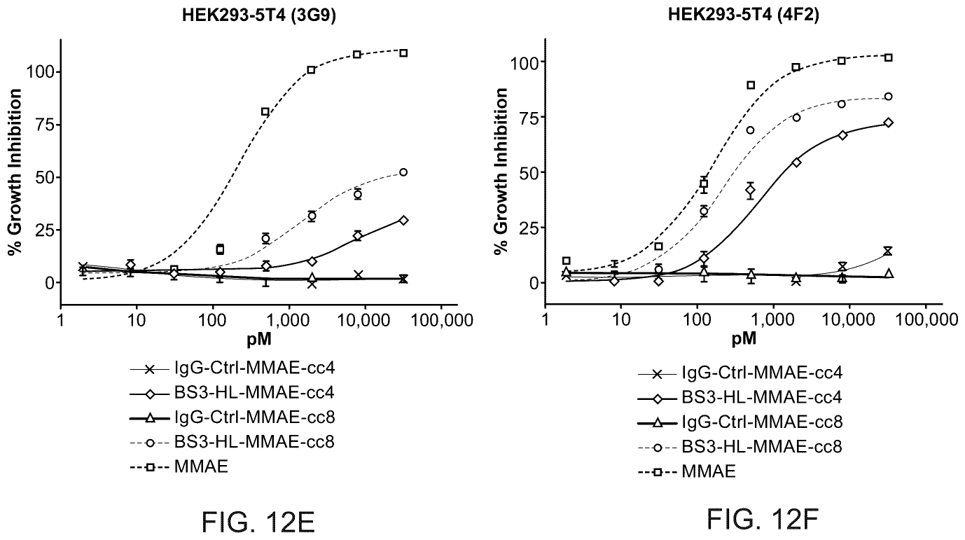

To evaluate the in vitro cytotoxic activity of an anti-5T4 bispecific antibody-drug conjugate, a panel of human cancer cell lines expressing different levels of 5T4 was selected and their 5T4 expression was verified by quantitative flow cytometry as described above. Mc-Val-Cit-MMAE was used as a payload with a drug-to-antibody ratio (DAR) of about 4 or 8. (indicated as cc4 or cc8 in FIGS. 11A-11F and FIGS. 12A-12F). Cells were treated with the biparatopic antibody-drug conjugates (ADCs) (Bs3-HL-MMAE-004, Bs3-HL-MMAE-008), IgG control ADCs (IgG-Ctrl-MMAE-001, IgG-Ctrl-MMAE-008) or the free warhead MMAE and then examined for cell viability (FIGS. 11A-11F) as well as growth inhibition (FIGS. 12A-12F).

FIGS. 11A-11F and 12A-12F depict representative data derived from different cell lines showing the mean percent viability ±SEM (n=3) (FIGS. 11A-11F) and mean percent growth inhibition ±SEM (n=3) (FIGS. 12A-12F) in 5T4-negative AGS gastric cancer cells (FIGS. 11A and 12A) and 5T4-positive cells including DU145 prostate cancer cells (FIGS. 11B and 12B), T-47D breast cancer cells (FIGS. 11C and 12D), MCF7 breast cancer cells (FIGS. 11D and 12C) and 5T4-transfected HEK293 cells—clones 3G9 (FIGS. 11E and 12E) and 4F2 (FIGS. 11F and 12F). Bispecific 5T4 ADCs showed 5T4-dependent and DAR-dependent cytotoxic and growth inhibition activities against 5T4-positive cells.

As shown in FIGS. 11A-11F and 12A-12F, the anti-5T4 bispecific ADCs did not induce significant cell death or growth inhibition in AGS, a cancer cell line negative for 5T4 expression. In two different stable clones (3G9 and 4F2) from 5T4-transfected HEK293 cells, the cell killing activity and growth inhibition were correlated to the level of 5T4 expression on the cell surface as well as DAR. Anti-5T4 bispecific antibody-drug conjugates induced cell killing activity and growth inhibition were generally confirmed in select 5T4 overexpressing cancer cell lines DU145, T-47D and MCF7. These in vitro data showed that the cytotoxic and growth inhibition activities by anti-5T4 bispecific antibody-drug conjugates were 5T4-dependent and DAR-dependent, suggesting that a certain level of 5T4 on the cell surface was required to mediate the intracellular delivery of sufficient payloads to induce cell death.

### Example 3: Suppression of Tumor Growth and Prolonged Survival in Multiple Tumor Models

FIGS. 13A-13B and 14A-14C show graphs of tumor growth and mouse survival using an in vivo model with immune compromised mice that have been implanted with human CDX tumors and then treated with an anti-5T4 bispecific antibody (BS3 configuration) conjugated to MMAE (1 mg/kg, 2 mg/kg, 3 mg/kg, 5 mg/kg or 10 mg/kg), compared to isotype control antibody conjugated to MMAE or mock PBS treatment. Anti-5T4 bispecific antibody-drug conjugate treatment of tumor bearing mice implanted with NCI-H1975 lung adenocarcinoma cells (FIGS. 13A-13B), MDA-MD-361 breast cancer cells (FIG. 14A), DU145 prostate carcinoma cells (FIG. 14B), and A549 lung carcinoma (FIG. 14C) resulted in diminished tumor growth in a dose dependent manner in multiple tumor models. As shown in FIG. 13B, treatment with anti-5T4 ADC extended probability of survival compared to controls.

These data show that 5T4 bispecific antibody-drug conjugates of the present disclosure can suppress tumor growth and prolong survival in multiple tumor models.

### Example 4: Increased Binding Affinity in Bispecific Antibodies Compared to Parental Antibodies

The binding affinity to 5T4 of bispecific antibodies compared to parental antibodies was evaluated again using Biacore SPR assays after optimization of the assay. The 5T4 biparatopic antibody contains binding domains derived from two 5T4 monoclonal antibodies, (m)Ab1 and (m)Ab2, recognizing distinct epitopes.

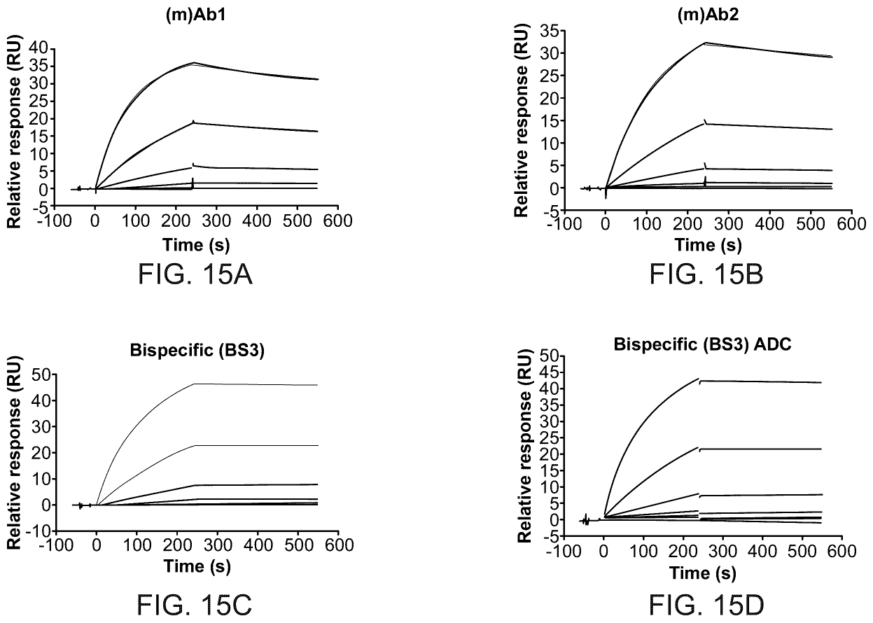

FIGS. 15A-15D show sensorgrams resulting from the optimized Biacore SPR assays. SPR kinetic experiments were performed on Biacore 8K systems (GE healthcare) with HBS-EP+ as running buffer. Bispecific 5T4-BS3 antibody and parental monospecific antibodies were captured in the active flow cells of a protein A sensor chip (Cytiva). Capture level for each antibody was maintained between 50-100 RU. The final analyte binding level at the highest concentration was maintained below 50 RU. For kinetic analysis, recombinant 5T4 protein and other testing samples were serially diluted to 6 different concentrations, ranging from 0.02 to 5 μg/mL (rather than 5 dilutions ranging from 10 to 0.625 nM, as in the initial SPR experiment described in Examples 1 and 2), and the serial dilutions were injected sequentially over both reference and active flow cells in each single channel. After each binding cycle, the sensor chip was regenerated with glycine solution (pH 1.5). The resulting sensorgrams were fitted with a one-to-one binding model to extract kinetic constants using Biacore 8K Evaluation Software.

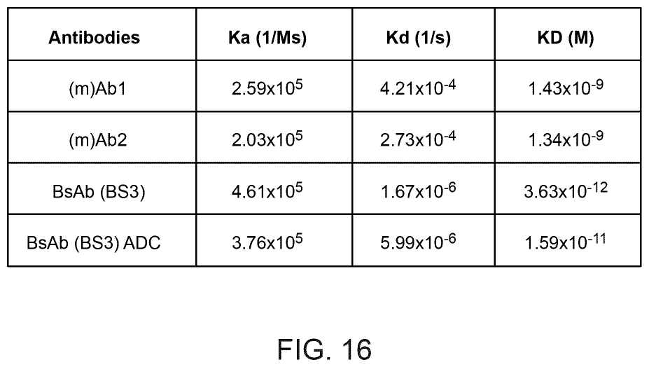

As shown in FIG. 16, the KD of the parental 5T4 monospecific antibodies, Ab1 and Ab2, was determined to be 1.43 nM and 1.34 nM, respectively. In contrast, the KD of the bispecific antibody, without conjugation to MMAE and with conjugated MMAE, was determined to be 3.63 μM and 15.9 μM, respectively.

These data show that 5T4 bispecific antibodies have greater binding affinity for 5T4 as compared to the parental monospecific antibodies from which they are derived.

1. An antibody-drug conjugate, comprising:
a. a first antigen binding domain that specifically binds to a first 5T4 epitope;
b. a second antigen binding domain that specifically binds to a second 5T4 epitope that is not the same as the first 5T4 epitope; and
c. a chemotherapeutic agent;
wherein the first antigen binding domain is operably linked to the second antigen binding domain.

2. The antibody-drug conjugate of claim 1, wherein the first and second antigen binding domains are independently selected from the group consisting of a Fab fragment, a F(ab′)₂ fragment, a scFv, a scab, a dAb, a single domain heavy chain antibody, and a single domain light chain antibody.

3. The antibody-drug conjugate of claim 1, comprising a full-length IgG antibody comprising the first antigen binding domain, and wherein the second antigen binding domain comprises a scFv.

4. (canceled)

5. The antibody-drug conjugate of claim 3, wherein the full-length IgG antibody comprises two heavy chains and two light chains.

6. The antibody-drug conjugate of claim 1, wherein the antibody-drug conjugate comprises two second antigen binding domain scFv that both specifically bind the second 5T4 epitope.

7.-8. (canceled)

9. The antibody-drug conjugate of claim 6, wherein:
(i) the N-terminus of the second antigen binding domain is operably linked to the C-terminus of a heavy chain of the full length IgG antibody comprising the first antigen binding domain,
(ii) the C-terminus of the second antigen binding domain is operably linked to the N-terminus of a heavy chain of the full length IgG antibody, or
(iii) the second antigen binding domain is operably linked to the heavy chain of the full length IgG antibody using a linker.

10.-12. (canceled)

13. The antibody-drug conjugate of claim 5, wherein the full length IgG antibody heavy chains comprise a heavy chain variable region domain and a heavy chain constant region domain, and/or
wherein the full length IgG antibody light chains comprise a light chain variable region domain and a light chain constant region domain.

14.-16. (canceled)

17. The antibody-drug conjugate of claim 13, wherein the heavy chain constant region domain comprises at least one mutation that reduces effector function, extends half-life, or a combination thereof.

18. The antibody-drug conjugate of claim 17, wherein:
(i) at least one mutation comprises an F at position 237 relative to SEQ ID NO: 100 (L234F), a C or A at position 242 relative to SEQ ID NO: 100 (S239C/A), an A at position 437 relative to SEQ ID NO: 100 (N434A), or a combination thereof, or
(ii) at least one mutation comprises an F at position 237 relative to SEQ ID NO: 100 (L234F), a C or A at position 242 relative to SEQ ID NO: 100 (S239C/A), and an A at position 437 relative to SEQ ID NO: 100 (N434A).

19. (canceled)

20. The antibody-drug conjugate of claim 1, comprising a full-length IgG antibody comprising the first antigen binding domain, and wherein the second antigen binding domain comprises an scFv, and the antibody-drug conjugate comprises four polypeptides comprising:
(a) two polypeptides comprising, from N to C terminus, (i) the full-length IgG antibody heavy chain, a linker, and the second antigen binding domain, or (ii) the second antigen binding domain, a linker, and the full-length IgG antibody; and
(b) two polypeptides comprising the full-length IgG antibody light chain.

21.-24. (canceled)

25. The antibody-drug conjugate of claim 1, wherein the first antigen binding domain comprises a heavy chain variable region domain comprising:
a. a HC CDR1 sequence comprising an amino acid sequence of SEQ ID NO: 1, or a sequence having 1, 2 or 3 substitutions, insertions or deletions relative thereto;
b. a HC CDR2 sequence comprising an amino acid sequence of SEQ ID NO: 2, or a sequence having 1, 2 or 3 substitutions, insertions or deletions relative thereto;
c. a HC CDR3 sequence comprising an amino acid sequence of SEQ ID NO: 3, or a sequence having 1, 2 or 3 substitutions, insertions or deletions relative thereto; and
wherein the first antigen binding domain comprises a light chain variable region domain comprising:
a. a LC CDR1 sequence comprising an amino acid sequence of SEQ ID NO: 7 or a sequence having 1, 2 or 3 substitutions, insertions or deletions relative thereto;
b. a LC CDR2 sequence comprising an amino acid sequence of SEQ ID NO: 8 or a sequence having 1, 2 or 3 substitutions, insertions or deletions relative thereto; and
c. a LC CDR3 sequence comprising an amino acid sequence of SEQ ID NO: 9 or a sequence having 1, 2 or 3 substitutions, insertions or deletions relative thereto.

26. The antibody-drug conjugate of claim 1, wherein the second antigen binding domain comprises a heavy chain variable region domain comprising:
a. a HC CDR1 sequence comprising an amino acid sequence of SEQ ID NO: 4, or a sequence having 1, 2 or 3 substitutions, insertions or deletions relative thereto;
b. a HC CDR2 sequence comprising an amino acid sequence of SEQ ID NO: 5, or a sequence having 1, 2 or 3 substitutions, insertions or deletions relative thereto;
c. a HC CDR3 sequence comprising an amino acid sequence of SEQ ID NO: 6, or a sequence having 1, 2 or 3 substitutions, insertions or deletions relative thereto; and
wherein the second antigen binding domain comprises a light chain variable region domain comprising:
a. a LC CDR1 sequence comprising an amino acid sequence of SEQ ID NO: 10, or a sequence having 1, 2 or 3 substitutions, insertions or deletions relative thereto;
b. a LC CDR2 sequence comprising an amino acid sequence of SEQ ID NO: 11, or a sequence having 1, 2 or 3 substitutions, insertions or deletions relative thereto; and
c. a LC CDR3 sequence comprising an amino acid sequence of SEQ ID NO: 12, or a sequence having 1, 2 or 3 substitutions, insertions or deletions relative thereto.

27. (canceled)

28. The antibody-drug conjugate of claim 1, wherein the first and/or second antigen binding domain comprises a heavy chain variable region domain comprising an amino acid sequence of SEQ ID NO: 96 or 97, and a light chain variable region domain comprising an amino acid sequence of SEQ ID NO: 98 or 99.

29. The antibody-drug conjugate of claim 1, wherein:
the first antigen binding domain comprises
a. a heavy chain variable region domain comprising an amino acid sequence of SEQ ID NO: 96, or a sequence having at least 80% identity thereto; and
b. a light chain variable region domain comprising an amino acid sequence of SEQ ID NO: 98, or a sequence having at least 80% identity thereto; and
wherein the second antigen binding domain comprises:
a. a heavy chain variable region domain comprising an amino acid sequence of SEQ ID 97, or a sequence having at least 80% identity thereto; and
b. a light chain variable region domain comprising an amino acid sequence of SEQ ID NO: 99, or a sequence having at least 80% identity thereto.

30. The antibody-drug conjugate of claim 1, comprising a full-length IgG antibody comprising the first antigen binding domain, and wherein the antibody-drug conjugate comprises:
two polypeptides comprising a first antigen binding domain heavy chain variable region domain comprising a sequence of SEQ ID NO: 96, a first linker comprising a sequence of SEQ ID NO: 152, a second antigen binding domain heavy chain variable region domain comprising a sequence of SEQ ID NO: 97, a second linker comprising a sequence of SEQ ID NO: 153, and a second antigen binding domain light chain variable region comprising a sequence of SEQ ID NO: 99, and
two polypeptides comprising a second antigen binding domain light chain variable region domain comprising a sequence of SEQ ID NO: 98.

31.-33. (canceled)

34. The antibody-drug conjugate of claim 1, comprising a full-length IgG antibody comprising the first antigen binding domain, and wherein the second antigen binding domain comprises a scFv, and the antibody-drug conjugate comprises four polypeptides comprising:
(a) two polypeptides comprising, from N to C terminus,
a first antigen binding domain heavy chain variable region domain comprising a sequence of SEQ ID NO: 96, an IgG1 isotype constant region domain comprising a sequence of SEQ ID NO: 148, a first linker comprising a sequence of SEQ ID NO: 152, a second antigen binding domain heavy chain variable region domain comprising a sequence of SEQ ID NO: 97, a second linker comprising a sequence of SEQ ID NO: 153, and a second antigen binding domain light chain variable region comprising a sequence of SEQ ID NO: 99;
(b) two polypeptides comprising, from N to C terminus, a first antigen binding domain variable light chain domain comprising a sequence of SEQ ID NO: 98, and
a light chain constant region domain comprising a sequence of SEQ ID NO: 149.

35. The antibody-drug conjugate of claim 1, comprising a full-length IgG antibody comprising the first antigen binding domain, and wherein the second antigen binding domain comprises a scFv, and the antibody-drug conjugate comprises four polypeptides comprising:
(a) two polypeptides comprising a sequence of SEQ ID NO: 100, or a sequence having at least 80% identity thereto; and
(b) two polypeptides comprising a sequence of SEQ ID NO: 101, or a sequence having at least 80% identity thereto.

36. The antibody-drug conjugate of claim 1, wherein the chemotherapeutic agent is conjugated to at least one of a full-length IgG antibody comprising the first antigen binding domain or the second antigen binding domain via a linker.

37. The antibody-drug conjugate of claim 1, wherein the chemotherapeutic agent is an auristatin.

38.-49. (canceled)

50. A nucleic acid system encoding the bispecific antibody comprising:
a. a first antigen binding domain that specifically binds to a first 5T4 epitope;
b. a second antigen binding domain that specifically binds to a second 5T4 epitope that is not the same as the first 5T4 epitope;
wherein the first antigen binding domain is operably linked to the second antigen binding domain.

51. One or more vectors comprising the nucleic acid system of claim 50.

52. (canceled)

53. A pharmaceutical composition comprising the antibody-drug conjugate of claim 1, and a pharmaceutically acceptable carrier, diluent, or excipient.

54. A method of making the antibody-drug conjugate, comprising:
a. contacting a plurality of cells with the nucleic acid system of claim 50;
b. culturing the plurality of cells under conditions whereby the bispecific antibody is expressed by at least one cell of the plurality of cells;
c. purifying the bispecific antibody; and
d. conjugating the bispecific antibody to a chemotherapeutic agent.

55.-57. (canceled)

58. A method of treating cancer in a subject in need thereof, the method comprising administering to a subject in need thereof a therapeutically effective amount of an antibody-drug conjugate, wherein the antibody-drug conjugate comprises:
a. a first antigen binding domain that specifically binds to a first 5T4 epitope;
b. a second antigen binding domain that specifically binds to a second 5T4 epitope that is not the same as the first 5T4 epitope; and
c. a chemotherapeutic agent;
wherein the first antigen binding domain is operably linked to the second antigen binding domain.

59.-68. (canceled)

69. A kit, comprising the antibody-drug conjugate of claim 1.

70. An antigen binding domain comprising a heavy chain variable region domain and a light chain variable region domain;
wherein the heavy chain variable region domain comprises:
a. a heavy chain (HC) complementarity determining region (CDR1) sequence selected from the group consisting of SEQ ID NOs: 1, 4, and 13-23, or a sequence having 1, 2 or 3 substitutions, insertions or deletions relative thereto;
b. a HC CDR2 sequence selected from the group consisting of SEQ ID NOs: 2, 5, and 24-39, or a sequence having 1, 2 or 3 substitutions, insertions or deletions relative thereto; and
c. a HC CDR3 sequence selected from the group consisting of SEQ ID NOs: 3, 6, and 40-52, or a sequence having 1, 2 or 3 substitutions, insertions or deletions relative thereto; and
wherein the light chain variable region domain comprises:
a. a light chain (LC) complementarity determining region (CDR1) sequence selected from the group consisting of SEQ ID NOs: 7, 10, and 53-66, or a sequence having 1, 2 or 3 substitutions, insertions or deletions relative thereto;
b. a LC CDR2 sequence selected from the group consisting of SEQ ID NOs: 8, 11, and 67-75, or a sequence having 1, 2 or 3 substitutions, insertions or deletions relative thereto; and
c. a LC CDR3 sequence selected from the group consisting of SEQ ID NOs: 9, 12, and 76-83, or a sequence having 1, 2 or 3 substitutions, insertions or deletions relative thereto.

71.-85. (canceled)


SEQUENCE LISTING (173 sequences)

SEQ ID NO: 1 - protein, 10 aa, synthetic construct
GYSFTDYYMH

SEQ ID NO: 2 - protein, 17 aa, synthetic construct
RVSPNNGATNTNQKFKD

SEQ ID NO: 3 - protein, 11 aa, synthetic construct
STMITSYYFDY

SEQ ID NO: 4 - protein, 10 aa, synthetic construct
GFDFSRYWMT

SEQ ID NO: 5 - protein, 17 aa, synthetic construct
EINPDSRTINYTPSLKD

SEQ ID NO: 6 - protein, 12 aa, synthetic construct
PDHDYNPYYFNY

SEQ ID NO: 7 - protein, 11 aa, synthetic construct
KASQSVSTDVA

SEQ ID NO: 8 - protein, 7 aa, synthetic construct
FASDRYT

SEQ ID NO: 9 - protein, 9 aa, synthetic construct
QQDYSSPPT

SEQ ID NO: 10 - protein, 15 aa, synthetic construct
RPSQSVSTSSNSYIH

SEQ ID NO: 11 - protein, 7 aa, synthetic construct
YASNLES

SEQ ID NO: 12 - protein, 9 aa, synthetic construct
QQSWEIPLT

SEQ ID NO: 13 - protein, 10 aa, synthetic construct
GFTFSSFGLH

SEQ ID NO: 14 - protein, 10 aa, synthetic construct
GFNIKDTYMH

SEQ ID NO: 15 - protein, 10 aa, synthetic construct
GYSFTGYYLH

SEQ ID NO: 16 - protein, 10 aa, synthetic construct
GYTFSSYWIE

SEQ ID NO: 17 - protein, 10 aa, synthetic construct
GYTFTHYVIS

SEQ ID NO: 18 - protein, 12 aa, synthetic construct
GFSLSTSGVGVD

SEQ ID NO: 19 - protein, 10 aa, synthetic construct
GFTFSNFGMH

SEQ ID NO: 20 - protein, 10 aa, synthetic construct
GFTFSSFGMH

SEQ ID NO: 21 - protein, 10 aa, synthetic construct
GYSFTGYYIH

SEQ ID NO: 22 - protein, 10 aa, synthetic construct
GYTFSTYWIE

SEQ ID NO: 23 - protein, 12 aa, synthetic construct
GFSLSTSGMGVG

SEQ ID NO: 24 - protein, 17 aa, synthetic construct
EINPDSNTINYTPSLKD

SEQ ID NO: 25 - protein, 17 aa, synthetic construct
YISVGSSTIYYADPVKG

SEQ ID NO: 26 - protein, 17 aa, synthetic construct
RIDPADGNTKFDPKFQG

SEQ ID NO: 27 - protein, 420 aa, Homo sapiens
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDV

SEQ ID NO: 28 - protein, 17 aa, synthetic construct
RVNPNNGATVYNQNFKG

SEQ ID NO: 29 - protein, 17 aa, synthetic construct
RVNPNNGGTIYNQNFKG

SEQ ID NO: 30 - protein, 17 aa, synthetic construct
EILPGSGRTNYNEKFKG

SEQ ID NO: 31 - protein, 17 aa, synthetic construct
EIYPGSGSTYYNEKFKG

SEQ ID NO: 32 - protein, 16 aa, synthetic construct
HIWWDDVKRYNPALKS

SEQ ID NO: 33 - protein, 17 aa, synthetic construct
YISSGTSTIYYADTVKG

SEQ ID NO: 34 - protein, 17 aa, synthetic construct
YISSGGSNIYYADTVKG

SEQ ID NO: 35 - protein, 17 aa, synthetic construct
EINPDSRTINYTPPLKD

SEQ ID NO: 36 - protein, 17 aa, synthetic construct
RIDPANGNTKYDPKFQG

SEQ ID NO: 37 - protein, 17 aa, synthetic construct
RVNPNNGGTSYNQKFKG

SEQ ID NO: 38 - protein, 17 aa, synthetic construct
RVNPNNGGTIYNQKFKG

SEQ ID NO: 39 - protein, 17 aa, synthetic construct
EILPGSGRNNYNEKFKG

SEQ ID NO: 40 - protein, 12 aa, synthetic construct
PDYDYNPYYFAY

SEQ ID NO: 41 - protein, 11 aa, synthetic construct
SRTYYRSEIDS

SEQ ID NO: 42 - protein, 10 aa, synthetic construct
FRRYDYVMDY

SEQ ID NO: 43 - protein, 11 aa, synthetic construct
SIMITTYDFDY

SEQ ID NO: 44 - protein, 11 aa, synthetic construct
SIMITTFDFDY

SEQ ID NO: 45 - protein, 12 aa, synthetic construct
GNYGSSPYYFDY

SEQ ID NO: 46 - protein, 8 aa, synthetic construct
GGRYGFDY

SEQ ID NO: 47 - protein, 12 aa, synthetic construct
IADGYSAPWFAY

SEQ ID NO: 48 - protein, 11 aa, synthetic construct
SRAYYRYEIDH

SEQ ID NO: 49 - protein, 10 aa, synthetic construct
WGDSYRYFDV

SEQ ID NO: 50 - protein, 12 aa, synthetic construct
PDHEYNPYYFNN

SEQ ID NO: 51 - protein, 10 aa, synthetic construct
FRRYALVMDY

SEQ ID NO: 52 - protein, 12 aa, synthetic construct
IADGYYAPWFAY

SEQ ID NO: 53 - protein, 15 aa, synthetic construct
RASQSVSTSRYSYMH

SEQ ID NO: 54 - protein, 15 aa, synthetic construct
RASQSVSSSSYNYMH

SEQ ID NO: 55 - protein, 15 aa, synthetic construct
RASESVDSYGNIFMH

SEQ ID NO: 56 - protein, 11 aa, synthetic construct
KASQDVSIDVG

SEQ ID NO: 57 - protein, 11 aa, synthetic construct
KASQDVNTAVV

SEQ ID NO: 58 - protein, 15 aa, synthetic construct
RASQSVSTSRNSYMH

SEQ ID NO: 59 - protein, 12 aa, synthetic construct
TASSSVSSSYLH

SEQ ID NO: 60 - protein, 11 aa, synthetic construct
KASQDINKYIA

SEQ ID NO: 61 - protein, 16 aa, synthetic construct
RASQSVSTSSSNSYVH

SEQ ID NO: 62 - protein, 10 aa, synthetic construct
SASSSVSFMH

SEQ ID NO: 63 - protein, 15 aa, synthetic construct
RASQSVSTSRYSYIH

SEQ ID NO: 64 - protein, 15 aa, synthetic construct
RASETIDSYGNTFMH

SEQ ID NO: 65 - protein, 11 aa, synthetic construct
KASQSVSNDVA

SEQ ID NO: 66 - protein, 11 aa, synthetic construct
KASQSVNYDVA

SEQ ID NO: 67 - protein, 7 aa, synthetic construct
SASTLES

SEQ ID NO: 68 - protein, 7 aa, synthetic construct
LASNLES

SEQ ID NO: 69 - protein, 7 aa, synthetic construct
WASTRHT

SEQ ID NO: 70 - protein, 7 aa, synthetic construct
STSNLAS

SEQ ID NO: 71 - protein, 7 aa, synthetic construct
YTSTLQP

SEQ ID NO: 72 - protein, 7 aa, synthetic construct
NASNLES

SEQ ID NO: 73 - protein, 7 aa, synthetic construct
YASNLEP

SEQ ID NO: 74 - protein, 7 aa, synthetic construct
YVSNRYI

SEQ ID NO: 75 - protein, 7 aa, synthetic construct
YASKRYT

SEQ ID NO: 76 - protein, 9 aa, synthetic construct
QHSWEIPLT

SEQ ID NO: 77 - protein, 9 aa, synthetic construct
QHSWEIPYT

SEQ ID NO: 78 - protein, 9 aa, synthetic construct
QQNNEDPWT

SEQ ID NO: 79 - protein, 9 aa, synthetic construct
QQHYSTPPT

SEQ ID NO: 80 - protein, 9 aa, synthetic construct
HQYHRSPLT

SEQ ID NO: 81 - protein, 8 aa, synthetic construct
LQCDNLWT

SEQ ID NO: 82 - protein, 11 aa, synthetic construct
QQRSGYPPKFT

SEQ ID NO: 83 - protein, 8 aa, synthetic construct
LQCDDLWT

SEQ ID NO: 84 - DNA, 31 bp, synthetic construct
  source 1..31: mol_type = other DNA
ggctacagcttcacagactactacatgcact

SEQ ID NO: 85 - DNA, 51 bp, synthetic construct
  source 1..51: mol_type = other DNA
cgagtgtctcccaacaatggagccaccaataccaaccagaagttcaaggat

SEQ ID NO: 86 - DNA, 34 bp, synthetic construct
  source 1..34: mol_type = other DNA
agtaccatgataacgtcctattacttcgactatt

SEQ ID NO: 87 - DNA, 30 bp, synthetic construct
  source 1..30: mol_type = other DNA
ggcttcgacttctcccggtattggatgacc

SEQ ID NO: 88 - DNA, 51 bp, synthetic construct
  source 1..51: mol_type = other DNA
gagatcaaccccgacagccggaccatcaactacacccctagcctgaaggac

SEQ ID NO: 89 - DNA, 34 bp, synthetic construct
  source 1..34: mol_type = other DNA
tgaccacgactacaacccctactacttcaactac

SEQ ID NO: 90 - DNA, 30 bp, synthetic construct
  source 1..30: mol_type = other DNA
gccagccaatcagtcagcaccgatgtagcc

SEQ ID NO: 91 - DNA, 18 bp, synthetic construct
  source 1..18: mol_type = other DNA
gcaagcgacaggtacact

SEQ ID NO: 92 - DNA, 27 bp, synthetic construct
  source 1..27: mol_type = other DNA
cagcaggactattcctctcctcctacc

SEQ ID NO: 93 - DNA, 45 bp, synthetic construct
  source 1..45: mol_type = other DNA
cggccttctcagtccgtgtccacctcctccaactcctacatccac

SEQ ID NO: 94 - DNA, 21 bp, synthetic construct
  source 1..21: mol_type = other DNA
tacgcctccaacctggaatcc

SEQ ID NO: 95 - DNA, 27 bp, synthetic construct
  source 1..27: mol_type = other DNA
cagcagtcctgggagatccctctgacc

SEQ ID NO: 96 - protein, 120 aa, synthetic construct
QVQLVQSGAEVKKPGASVKVSCKASGYSFTDYYMHWVRQAPGQGLEWMGRVSPNNGATNTNQKFKDRVTMTRDTSISTAYMELRSLRSEDTAVYYCARSTMITSYYFDYWGQGTLVTVSS

SEQ ID NO: 97 - protein, 121 aa, synthetic construct
EVQLLESGGGLVQPGGSLRLSCAASGFDFSRYWMTWVRQAPGKCLEWVSEINPDSRTINYTPSLKDRFTISRDNAKNTLYLQMNSLRAEDTAVYYCARPDHDYNPYYFNYWGQGTTVTVSS

SEQ ID NO: 98 - protein, 107 aa, synthetic construct
DIVMTQSPSFLSVSVGDRVTITCKASQSVSTDVAWYQQKPGQAPKLLIYFASDRYTGVPDRFSGSGSGTDFTFTISSLQAEDVAVYYCQQDYSSPPTFGGGTKLEIK

SEQ ID NO: 99 - protein, 111 aa, synthetic construct
DIVLTQSPASLAVSLGQRATISCRPSQSVSTSSNSYIHWYQQKPGQPPKLLIKYASNLESGVPARFSGSGSGTDFTLTISPVEAEDTATYYCQQSWEIPLTFGCGTKLEIK

SEQ ID NO: 100 - protein, 717 aa, synthetic construct
QVQLVQSGAEVKKPGASVKVSCKASGYSFTDYYMHWVRQAPGQGLEWMGRVSPNNGATNTNQKFKDRVTMTRDTSISTAYMELRSLRSEDTAVYYCARSTMITSYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPGKGGGGSGGGGSGGGGSEVQLLESGGGLVQPGGSLRLSCAASGFDFSRYWMTWVRQAPGKCLEWVSEINPDSRTINYTPSLKDRFTISRDNAKNTLYLQMNSLRAEDTAVYYCARPDHDYNPYYFNYWGQGTTVTVSSGGGGSGGGGSGGGGSGGGGSDIVLTQSPASLAVSLGQRATISCRPSQSVSTSSNSYIHWYQQKPGQPPKLLIKYASNLESGVPARFSGSGSGTDFTLTISPVEAEDTATYYCQQSWEIPLTFGCGTKLEIK

SEQ ID NO: 101 - protein, 214 aa, synthetic construct
DIVMTQSPSFLSVSVGDRVTITCKASQSVSTDVAWYQQKPGQAPKLLIYFASDRYTGVPDRFSGSGSGTDFTFTISSLQAEDVAVYYCQQDYSSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 121 aa, synthetic construct
EVQLLESGGGLVQPGGSLRLSCAASGFDFSRYWMTWVRQAPGKGLEWVSEINPDSRTINYTPSLKDRFTISRDNAKNTLYLQMNSLRAEDTAVYYCARPDHDYNPYYFNYWGQGTTVTVSS

SEQ ID NO: 103 - protein, 111 aa, synthetic construct
DIVLTQSPASLAVSLGQRATISCRPSQSVSTSSNSYIHWYQQKPGQPPKLLIKYASNLESGVPARFSGSGSGTDFTLTISPVEAEDTATYYCQQSWEIPLTFGGGTKLEIK

SEQ ID NO: 104 - protein, 121 aa, synthetic construct
EVQLLESGGGLVQPGGSLRLSCAASGFDFSRYWMTWVRQAPGKGLEWVSEINPDSNTINYTPSLKDRFTISRDNAKNTLYLQMNSLRAEDTAVYYCASPDYDYNPYYFAYWGQGTLVTVSS

SEQ ID NO: 105 - protein, 111 aa, synthetic construct
DIVLTQSPASLAVSLGQRATISCRASQSVSTSRYSYMHWYQQKPGQPPKLLIKYASNLESGVPARFSGSGSGTDFTLTISSLQAEDFATYYCQHSWEIPLTFGGGTKLEIK

SEQ ID NO: 106 - protein, 120 aa, synthetic construct
EVQLVESGGGLVQPGGSLRLSCAASGFTFSSFGLHWVRQAPGKGLEWVAYISVGSSTIYYADPVKGRFTISRDNAKNTLYLQMNSLRAEDTAVYYCARSRTYYRSEIDSWGQGTTVTVSS

SEQ ID NO: 107 - protein, 111 aa, synthetic construct
DIVLTQSPASLAVSLGQRATISCRASQSVSSSSYNYMHWYQQKPGQPPKLLIYSASTLESGVPARFSGSGSGTDFTLTISSLQAEDFATYYCQHSWEIPYTFGQGTKLEIK

SEQ ID NO: 108 - protein, 119 aa, synthetic construct
EVQLVQSGAEVKKPGASVKVSCKASGFNIKDTYMHWVRQAPGQGLEWMGRIDPADGNTKFDPKFQGRVTITADTSTSTAYMELSSLRSEDTAVYYCARFRRYDYVMDYWGQGTTVTVSS

SEQ ID NO: 109 - protein, 111 aa, synthetic construct
DIVLTQSPASLAVSLGQRATITCRASESVDSYGNIFMHWYQQKPGQPPKLLIYLASNLESGVPARFSGSGSGTDFTLTISSLEAEDFATYYCQQNNEDPWTFGGGTKLEIK

SEQ ID NO: 110-111 - skipped (2 entries)

SEQ ID NO: 112 - protein, 120 aa, synthetic construct
EVQLVQSGAEVKKPGASVKVSCKASGYSFTGYYLHWVRQAPGKGLEWMGRVNPNNGATVYNQNFKGRVTMTRDKSISTAYMELRSLRSEDTAVYYCVRSIMITTYDFDYWGQGTLVTVSS

SEQ ID NO: 113 - protein, 107 aa, synthetic construct
DIVMTQSPSSLSASVGDRVTITCKASQDVSIDVGWYQQKPGQAPKLLIYWASTRHTGVPDRFSGSGSGTDFTLTISSLQPEDFATYYCQQHHIVPPTFGQGTKVEIK

SEQ ID NO: 114 - protein, 120 aa, synthetic construct
EVQLVQSGAEVKKPGASVKVSCKASGYSFTGYYLHWVRQAPGQCLEWMGRVNPNNGGTIYNQNFKGRVTMTRDKSISTAYMELRSLRSEDTAVYYCARSIMITTFDFDYWGQGTLVTVSS

SEQ ID NO: 115 - protein, 107 aa, synthetic construct
DIVMTQSPSSLSASVGDRVTITCKASQDVNTAVVWYQQKPGQAPELLIYWASTRHTGVPDRFSGSGSGTDYTLTISSLQAEDFATYYCQQHYSTPPTFGQGTKVEIK

SEQ ID NO: 116 - protein, 121 aa, synthetic construct
QVQLVQSGAEVKKPGASVKISCKASGYTFSSYWIEWVRQAPGQGLEWMGEILPGSGRTNYNEKFKGRVTITADTSTSTAYMELSSLRSEDTAVYYCARGNYGSSPYYFDYWGQGTLVTVSS

SEQ ID NO: 117 - protein, 111 aa, synthetic construct
DIVLTQSPASLAVSLGQRATISCRASQSVSTSRNSYMHWYQQKPGQPPKLLIKYASNLESGVPARFSGSGSGTDFTLTISSLEPEDFATYYCQHSWEIPLTFGGGTKLEIK

SEQ ID NO: 118 - protein, 117 aa, synthetic construct
QVQLVQSGAEVKKPGASVKVSCKASGYTFTHYVISWVRQATGQGLEWIGEIYPGSGSTYYNEKFKGRVTMTADKSISTAYMELSSLRSEDTAVYYCARGGRYGFDYWGQGTTVTVSS

SEQ ID NO: 119 - protein, 108 aa, synthetic construct
EIVLTQSPATLSASPGERVTLTCTASSSVSSSYLHWYQQKPGLAPKLLIYSTSNLASGVPARFSGSGSGTDYTLTISSLEPEDFATYYCHQYHRSPLTFGGGTKLEIK

SEQ ID NO: 120 - protein, 122 aa, synthetic construct
QVTLKESGPTLVKPTQTLTLTCTFSGFSLSTSGVGVDWIRQPPGKALEWLAHIWWDDVKRYNPALKSRLTISKDTSKNQVVLTMTNMDPVDTATYYCARIADGYSAPWFAYWGQGTLVTVSS

SEQ ID NO: 121 - protein, 106 aa, synthetic construct
DIQMTQSPSSLSASVGDRVTITCKASQDINKYIAWYQQKPGKAPKLLIYYTSTLQPGIPSRFSGSGSGTDFTFTISSLQPEDIATYYCLQCDNLWTFGGGTKLEIK

SEQ ID NO: 122 - protein, 120 aa, synthetic construct
DVQLVESGGGLVQPGGSRKLSCAASGFTFSNFGMHWVRQAPEKGLEWVAYISSGTSTIYYADTVKGRFTISRDNPKNTLFLQMTSLRSEDTAMYYCARSRAYYRYEIDHWGQGTTLTVSS

SEQ ID NO: 123 - protein, 112 aa, synthetic construct
DIVLTQSPASLAVSLGQRATISCRASQSVSTSSSNSYVHWYQQKLGQPPKLLIKNASNLESGVPARFSGSGSGTDFILNIHPVEEEDTATYYCQHSWEIPYTFGGGTKLEIK

SEQ ID NO: 124 - protein, 119 aa, synthetic construct
DVQLVESGGGLAQPGGSRKLSCEASGFTFSSFGMHWVRQPPEKGLEWVAYISSGGSNIYYADTVKGRFTISRDNPKNTLSLQMTSLRSEDTAMYYCARWGDSYRYFDVWGAGTTVTVSS

SEQ ID NO: 125 - protein, 108 aa, synthetic construct
QIVLTQSPAILSASPGEKVSITCSASSSVSFMHWFQQKPGTSPKLWIYSTSNLASGVPARFSGSGSGTSYSLTISRMEAEDAATYYCQQRSGYPPKFTFGSGTKLEIK

SEQ ID NO: 126 - protein, 121 aa, synthetic construct
EVKLLESGGGLVQPGGSLKLSCAASGFDFSRYWMTWVRQAPGKGLEWIGEINPDSRTINYTPPLKDKIIISRDNAKNTLYLQMNKVRSEDTALYYCARPDHEYNPYYFNNWGQGTTLTVSS

SEQ ID NO: 127 - protein, 111 aa, synthetic construct
DIVLTQSPASLPVSLGQRATISCRPSQSVSTSSNSYIHWYQQKPGQPPKLLIKYASNLEPGVPARFSGSGSGTDFTLNIHPVEEEDTATYYCQQSWEIPLTFGAGTKLELK

SEQ ID NO: 128 - protein, 121 aa, synthetic construct
EVKLLESGGGLVQPGGSLKLSCAASGFDFSRYWMTWVRQAPGKGLEWIGEINPDSRTINYTPSLKDKFIISRDNAKNTLYLHMSKVRSEDTALYYCARPDHDYNPYYFNYWGQGTTLTVSS

SEQ ID NO: 129 - protein, 111 aa, synthetic construct
DIVLTQSPASLAVSLGQRATISCRPSQSVSTSSNSYIHWYQQKPGQPPKLLIKYASNLESGVPARFSGSGSGTDFTLNIHPVEEEDTATYYCQQSWEIPLTFGAGTKLELK

SEQ ID NO: 130 - skipped

SEQ ID NO: 131 - protein, 111 aa, synthetic construct
DIVVTQSPASLAVSLGQRATISCRASQSVSTSRYSYIHWYQQKPGQSPKLLIKYASNLESGVPPRFSGSGSGTDFTLNIHPVEEEDTATYYCQHSWEIPLTFGAGTKLELK

SEQ ID NO: 132 - protein, 119 aa, synthetic construct
EVQLQQSGAELVKPGASVKLSCTASGFNIKDTYMHWVKQRPEQGLEWIGRIDPANGNTKYDPKFQGKATITSDTSSNTAYLQLSSLTSEDTAVYYCARFRRYALVMDYWGQGTSVTVSS

SEQ ID NO: 133 - protein, 111 aa, synthetic construct
NIVLTQSPASLAVSLGQRATISCRASETIDSYGNTFMHWYHQKPGQPPKLLIYLASNLESGVPARFSGSGSRTDFTLTIDPVEADDAATYYCQQNNEDPWTFGGGTKLEIK

SEQ ID NO: 134 - protein, 120 aa, synthetic construct
EVQLQQSGPDLVKPGASVRISCKASGYSFTGYYLHWVKQSHGESLEWIGRVNPNNGGTSYNQKFKGKAILTVDTSSNTVYMELRSLTSEDSAVYNCARSTMITSYYFDYWGQGTTLTVSS

SEQ ID NO: 135 - protein, 107 aa, synthetic construct
SIVMTQTPKFLLVSSGDRVTMTCKASQSVSNDVAWYQQKPGQSPKLLIYYVSNRYIGVPDRFTGSGYGTDFTFTISTVQAEDLAVYFCQQDYSSPPTFGGGTKLEIK

SEQ ID NO: 136 - protein, 120 aa, synthetic construct
EVQLQQSGPDLVKPGASVRISCKASGYSFTGYYIHWVKQSHGKSLEWIGRVNPNNGGTIYNQKFKGKAILTVDKSSNTASMEYRSLTSEDSAVYYCARSTMITSYYFDYWGQGTTLTVSS

SEQ ID NO: 137 - protein, 107 aa, synthetic construct
NIVMTQTPKFLLVSAGDRVTITCKASQSVNYDVAWYQQKPGQSPKPLIYYASKRYTGVPDRFTGSGFGTDFTFTINTVQAGDLAIYFCQQDYSSPPTFGGGTKLEIK

SEQ ID NO: 138 - protein, 121 aa, synthetic construct
QVQLQQSGAELMKPGASVRISCKATGYTFSTYWIEWVRQRPGHGLEWIGEILPGSGRNNYNEKFKGKATFTADTSSNTAYIQLSSLTSEDSAVYYCAKGNYGSSPYYFDYWGQGTTLTVSS

SEQ ID NO: 139 - protein, 111 aa, synthetic construct
DIVLTQSPASLVVSLGQRATISCRASQSVSTSRNSYMHWYQQKPGQPPKVLIKYASNLESGVPARFSGSGSGTDFTLNIHPVEEEDTATYYCQHSWEIPLTFGAGTKLELK

SEQ ID NO: 140 - protein, 122 aa, synthetic construct
QVTLKESGPGILQPSQTLSLTCSFSGFSLSTSGMGVGWIRQPSGKGLEWLAHIWWDDVKRYNPALKSRLTISKDTSSSQVFLNIASVDTADTATYYCARIADGYYAPWFAYWGQGTLVTVSA

SEQ ID NO: 141 - protein, 106 aa, synthetic construct
DIQMTQSPSSLSASLGGKVTITCKASQDINKYIAWYQHKPGKGPRLIIHYTSTLQPDIPSRFSGSGSGRDYSFSISNLEPEDIATYFCLQCDDLWTFGGGTKIEIK

SEQ ID NO: 142 - DNA, 2148 bp, synthetic construct
  source 1..2148: mol_type = other DNA
caggttcagctggtacagagcggagctgaagtcaagaaacctggggcttcagtgaaggttagctgcaaagcctctggctacagcttcacagactactacatgcactgggttagacaggcaccaggacaagggcttgagtggatgggtcgagtgtctcccaacaatggagccaccaataccaaccagaagttcaaggatcgtgtcaccatgactcgggatacctccatttccactgcctatatggaactgcgctccttgaggagtgaggacacagcagtgtactattgcgctaggagtaccatgataacgtcctattacttcgactattggggccaaggtactctggtgacagtgtctagcgcgtcgacaaagggcccctccgtgtttcctctggctccaagctctaagagcacctctggaggaacagccgctctgggatgtctggtgaaggattacttccctgagccagtgaccgtgagctggaactctggcgccctgacctccggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagttcctgggaggaccttgcgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgacctgtctggtgaagggcttctacccatctgacatcgctgtggagtgggagtccaatggccagcccgagaacaattataagaccacaccacccgtgctggactccgatggcagcttctttctgtactccaagctgaccgtggataagagcaggtggcagcagggcaacgtgttttcctgcagcgtgatgcacgaggccctgcacgctcattatacacagaaatctctgtccctgagcccaggcaagggtggagggggatccggcggcggcggcagtggcggaggaggctccgaggtgcagctgctggaatctggcggaggattggttcagcctggcggctctctgagactgtcttgtgccgcctctggcttcgacttctcccggtattggatgacctgggtccgacaggctcctggcaagtgtctggaatgggtgtccgagatcaaccccgacagccggaccatcaactacacccctagcctgaaggaccggttcaccatctccagagacaacgccaagaacaccctgtacctgcagatgaactccctgagagccgaggacaccgccgtgtactactgtgccagacctgaccacgactacaacccctactacttcaactactggggccagggcaccaccgtgacagtttctagcggcggaggtggaagcggaggcggaggtagtggtggtggcggatctggtggcggtggatctgatatcgtgctgacccagtctcctgccagcctggctgtttctctgggacagagagccaccatcagctgccggccttctcagtccgtgtccacctcctccaactcctacatccactggtatcagcagaagcccggccagcctcctaagctgattaagtacgcctccaacctggaatccggcgtgccagccagattttccggctctggctctggcaccgacttcaccctgacaatctctcccgtggaagctgaggataccgccacctactactgccagcagtcctgggagatccctctgacctttggctgtggcaccaagctggaaatcaaa

SEQ ID NO: 143 - DNA, 642 bp, synthetic construct
  source 1..642: mol_type = other DNA
gacatcgtgatgacacagtcaccctcctttctgagtgtgagtgttggggatagagtcaccatcacatgcaaagccagccaatcagtcagcaccgatgtagcctggtatcagcagaaaccaggccaagctcccaagctgctgatctacttcgcaagcgacaggtacactggtgtgccagatcggttttctgggtctggatctggtactgacttcaccttcacgatctccagtctgcaggctgaagatgttgccgtctactactgtcagcaggactattcctctcctcctacctttggaggaggcactaagctggagataaagcgtacggtggccgctccatccgtgttcatctttccaccctccgacgagcagctgaagtccggaaccgctagcgtggtgtgcctgctgaacaacttctacccaagagaggccaaggtgcagtggaaggtggataacgctctgcagagcggcaattctcaggagtccgtgaccgagcaggacagcaaggattctacatattccctgagctctaccctgacactgtccaaggccgattacgagaagcacaaggtgtatgcttgcgaggtgacccatcagggcctgtccagccccgtgacaaagagcttcaaccgcggcgagtgt

SEQ ID NO: 144 - DNA, 360 bp, synthetic construct
  source 1..360: mol_type = other DNA
caggttcagctggtacagagcggagctgaagtcaagaaacctggggcttcagtgaaggttagctgcaaagcctctggctacagcttcacagactactacatgcactgggttagacaggcaccaggacaagggcttgagtggatgggtcgagtgtctcccaacaatggagccaccaataccaaccagaagttcaaggatcgtgtcaccatgactcgggatacctccatttccactgcctatatggaactgcgctccttgaggagtgaggacacagcagtgtactattgcgctaggagtaccatgataacgtcctattacttcgactattggggccaaggtactctggtgacagtgtctagc

SEQ ID NO: 145 - DNA, 363 bp, synthetic construct
  source 1..363: mol_type = other DNA
gaggtgcagctgctggaatctggcggaggattggttcagcctggcggctctctgagactgtcttgtgccgcctctggcttcgacttctcccggtattggatgacctgggtccgacaggctcctggcaagtgtctggaatgggtgtccgagatcaaccccgacagccggaccatcaactacacccctagcctgaaggaccggttcaccatctccagagacaacgccaagaacaccctgtacctgcagatgaactccctgagagccgaggacaccgccgtgtactactgtgccagacctgaccacgactacaacccctactacttcaactactggggccagggcaccaccgtgacagtttctagc

SEQ ID NO: 146 - DNA, 322 bp, synthetic construct
  source 1..322: mol_type = other DNA
gacatcgtgatgacacagtcaccctcctttctgagtgtgagtgttggggatagagtcaccatcacatgcaaagccagccaatcagtcagcaccgatgtagcctggtatcagcagaaaccaggccaagctcccaagctgctgatctacttcgcaagcgacaggtacactggtgtgccagatcggttttctgggtctggatctggtactgacttcaccttcacgatctccagtctgcaggctgaagatgttgccgtctactactgtcagcaggactattcctctcctcctacctttggaggaggcactaagctggagataaagc

SEQ ID NO: 147 - DNA, 333 bp, synthetic construct
  source 1..333: mol_type = other DNA
gatatcgtgctgacccagtctcctgccagcctggctgtttctctgggacagagagccaccatcagctgccggccttctcagtccgtgtccacctcctccaactcctacatccactggtatcagcagaagcccggccagcctcctaagctgctgattaagtacgcctccaacctggaatccggcgtgccagccagattttccggctctggctctggcaccgacttcaccctgacaatctctcccgtggaagctgaggataccgccacctactactgccagcagtcctgggagatccctctgacctttggctgtggcaccaagctggaaatcaaa

SEQ ID NO: 148 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 149 - protein, 107 aa, synthetic construct [sequence omitted]

SEQ ID NO: 150 - DNA, 990 bp, synthetic construct
  source 1..990: mol_type = other DNA
gcgtcgacaaagggcccctccgtgtttcctctggctccaagctctaagagcacctctggaggaacagccgctctgggatgtctggtgaaggattacttccctgagccagtgaccgtgagctggaactctggcgccctgacctccggagtgcatacatttcccgctgtgctgcagtccagcggcctgtatagcctgtcttccgtggtgaccgtgcctagctcttccctgggcacccagacatacatctgcaacgtgaatcacaagccctccaatacaaaggtggacaagagagtggagcctaagagctgtgataagacccatacatgcccaccatgtccagctcctgagttcctgggaggaccttgcgtgttcctgtttcctccaaagccaaaggacaccctgatgatctctcgcacccctgaggtgacatgcgtggtggtggacgtgtcccacgaggatccagaggtgaagttcaactggtacgtggatggcgtggaggtgcataatgctaagaccaagcctagggaggagcagtacaacagcacctatcgggtggtgtctgtgctgacagtgctgcaccaggactggctgaacggcaaggagtacaagtgcaaggtgagcaataaggccctgccagctcccatcgagaagaccatctctaaggccaagggccagcccagagagcctcaggtgtatacactgccccctagccgcgaggagatgaccaagaaccaggtgtctctgacctgtctggtgaagggcttctacccatctgacatcgctgtggagtgggagtccaatggccagcccgagaacaattataagaccacaccacccgtgctggactccgatggcagcttctttctgtactccaagctgaccgtggataagagcaggtggcagcagggcaacgtgttttcctgcagcgtgatgcacgaggccctgcacgctcattatacacagaaatctctgtccctgagcccaggcaag

SEQ ID NO: 151 - DNA, 320 bp, synthetic construct
  source 1..320: mol_type = other DNA
gtacggtggccgctccatccgtgttcatctttccaccctccgacgagcagctgaagtccggaaccgctagcgtggtgtgcctgctgaacaacttctacccaagagaggccaaggtgcagtggaaggtggataacgctctgcagagcggcaattctcaggagtccgtgaccgagcaggacagcaaggattctacatattccctgagctctaccctgacactgtccaaggccgattacgagaagcacaaggtgtatgcttgcgaggtgacccatcagggcctgtccagccccgtgacaaagagcttcaaccgcggcgagtgt

SEQ ID NO: 152 - protein, 15 aa, synthetic construct
GGGGSGGGGSGGGGS

SEQ ID NO: 153 - DNA, 45 bp, synthetic construct
  source 1..45: mol_type = other DNA
ggtggagggggatccggcggcggcggcagtggcggaggaggctcc

SEQ ID NO: 154 - protein, 20 aa, synthetic construct
GGGGSGGGGSGGGGSGGGGS

SEQ ID NO: 155 - DNA, 60 bp, synthetic construct
  source 1..60: mol_type = other DNA
ggcggaggtggaagcggaggcggaggtagtggtggtggcggatctggtggcggtggatct

SEQ ID NO: 156 - protein, 121 aa, synthetic construct
EVKLLESGGGLVQPGGSLKLSCAASGFDFSRYWMTWVRQAPGKGLEWIGEINPDSNTINYTPSLKDKFIISRDNAKNTLYLQLTKVRSEDTGLYYCASPDYDYNPYYFAYWGQGTPLTVSS

SEQ ID NO: 157 - protein, 111 aa, synthetic construct
DIVVTQSPASLAVSLGQRATISCRASQSVSTSRYSYMHWYQQKPGQSPKLLIKYASNLESGVPPRFSGSGSGTDFTLNIHPVEEEDTATYYCQHSWEIPLTFGAGTKLELK

SEQ ID NO: 158 - protein, 120 aa, synthetic construct
DVQLVESGGGLVQPGGSRTLSCAASGFTFSSFGLHWVRQSPEKGLEWVAYISVGSSTIYYADPVKGRFTISRDNPKNTLFLQMTSLRSEDTAIYYCARSRTYYRSEIDSWGQGTTLTVSS

SEQ ID NO: 159 - protein, 111 aa, synthetic construct
DIVLTQSPASLTVSLGQRATISCRASQSVSSSSYNYMHWYRQKPGQPPKLLIKSASTLESGVPARFSGSGSGTDFTLNIHPVEEEDTATYYCQHSWEIPYTFGGGTKLEIK

SEQ ID NO: 160 - protein, 119 aa, synthetic construct
EVQLQQSGAELVKPGASVKLSCTASGFNIKDTYMHWVKQRPEQGLEWIGRIDPADGNTKFDPKFQGKATITADTSSNTAYLQLSSLTSEDTAVYYCARFRRYDYVMDYWGQGTSVTVSS

SEQ ID NO: 161 - protein, 111 aa, synthetic construct
NIVLTQSPASLAVSLGQRATISCRASESVDSYGNIFMHWYQQIPGQPPKLLIYLASNLESGVPARFSGSGSRTDFTLTIDPVEADDAATYYCQQNNEDPWTFGGGTKLEIK

SEQ ID NO: 162 - protein, 120 aa, synthetic construct
EVQLQQSGPDLVKPGASVKISCKASGYSFTDYYMHWVKQSHGKSLEWIGRVSPNNGATNTNQKFKDKAILTVDKSSSTAYMDLRSLTSEDSAVYYCARSTMITSYYFDYWGQGTTLTVSS

SEQ ID NO: 163 - protein, 107 aa, synthetic construct
SIVMTQTPKFLLVSAGDRITITCKASQSVSTDVAWYQQKPGQSPKLLIYFASDRYTGVPDRFTGSGNGTDFTFTITTVQAEDLAIYFCQQDYSSPPTFGGGTKLEIK

SEQ ID NO: 164 - protein, 120 aa, synthetic construct
EVQLQQSGPDLVKPGASVRISCKASGYSFTGYYLHWVKQSHGKSLEWIGRVNPNNGATVYNQNFKGKAVLTVDKSSSTAYMELRSLTSEDSAVYYCVRSIMITTYDFDYWGQGTTLTVSS

SEQ ID NO: 165 - protein, 107 aa, synthetic construct
DIVMTQSHKFMSTSVGDRVSITCKASQDVSIDVGWYQQKPGQSPKLLIYWASTRHTGVPDRFTGSGSGTDYTLTISNVQAEDLALYYCQQHHIVPPTFGSGTKLEIK

SEQ ID NO: 166 - protein, 120 aa, synthetic construct
EVQLQQSGPDLVRPGASVKISCKASGYSFTGYYLHWVKQSHGKSLEWIGRVNPNNGGTIYNQNFKGKAMLNVDKSSTTAYMELRSLTSEDSAVYYCARSIMITTFDFDYWGQGTTLTVSS

SEQ ID NO: 167 - protein, 107 aa, synthetic construct
DIVMTQSHKFMSTSVGDRVSITCKASQDVNTAVVWYQQKPGQSPELLIYWASTRHTGVPDRFSGSGSGTDYTLTISSVQAEDLALYYCQQHYSTPPTFGSGTKLEIK

SEQ ID NO: 168 - protein, 121 aa, synthetic construct
QVQLQQSGAELMKPGASVKISCKATGYTFSSYWIEWVRQRPGHGLEWIGEILPGSGRTNYNEKFKGKATFTADTSSNTAYIQLSSLTSEDSAVYYCAKGNYGSSPYYFDYWGQGTTLTVSS

SEQ ID NO: 169 - protein, 111 aa, synthetic construct
DIVLTQSPASLAVSLGQRATISCRASQSVSTSRNSYMHWYQQKPGQPPKVLIKYASNLESGVPARFSGSGSGTDFTLNIHPVEEEDTATYYCQHSWEIPLTFGAGTKLELK

SEQ ID NO: 170 - protein, 117 aa, synthetic construct
QVQLQQSGPELVKPGASVKMSCKASGYTFTHYVISWVKQRTGQGLEWIGEIYPGSGSTYYNEKFKGKATLTADKSSNTAYMQLSSLTSEDSAVYFCARGGRYGFDYWGQGTTLTVSS

SEQ ID NO: 171 - protein, 108 aa, synthetic construct
QIVLTQSPAIMSASLGERVTMTCTASSSVSSSYLHWYQQKPGSSPKLWIYSTSNLASGVPARFSGSGSGTSYSLTISSMEAEDAVTYYCHQYHRSPLTFGAGTKLELK

SEQ ID NO: 172 - protein, 122 aa, synthetic construct
QVTLKESGPGILQPSQTLSLTCSFSGFSLSTSGVGVDWIRQPSGKGLEWLAHIWWDDVKRYNPALKSRLTISKDTSSSQVFLKIASVDTADTATYYCARIADGYSAPWFAYWGQGTLVTVSA

SEQ ID NO: 173 - protein, 106 aa, synthetic construct
DIQMTQSPSSLSASLGGKVTITCKASQDINKYIAWYQHKPGKGPRLLIHYTSTLQPGIPSRFSGSGSGRDYSFSISNLEPEDIATYYCLQCDNLWTFGGGTKLEIK